(19) United States
(12) Patent Application Publication
(10) Pub. No.: US 2026/0284076 A1
(43) Pub. Date: Sep. 24, 2026

(54) TESTOSTERONE AND DIFLUOROMETHYLORNITHINE COMBINATION THERAPY FOR PROSTATE CANCER

(71) Applicant: The Johns Hopkins University, Baltimore, MD (US)

(72) Inventors: Samuel R. DENMEADE, Baltimore, MD (US); Laura A. SENA, Ellicott City, MD (US); Robert A. CASERO, White Hall, MD (US)

(21) Appl. No.: 19/474,469

(22) PCT Filed: May 10, 2024
(86) PCT No.: PCT/US2024/028823

§ 371 Date: Oct. 10, 2025

Related U.S. Application Data
(60) Provisional application No. 63/501,323, filed on May 10, 2023.
(60) Provisional application No. 63/635,897, filed on Apr. 18, 2024.

(51) Int. Cl. A61K 31/568; A61K 31/167; A61K 31/198; A61K 31/4155; A61K 31/4164; A61K 31/4166; A61K 31/4192; A61K 31/4439; A61K 31/45; A61K 31/496; A61K 31/573; A61K 31/58; A61K 38/09; A61P 33/00; C07K 16/28

(52) U.S. Cl. CPC A61K 31/568; A61K 31/167; A61K 31/198; A61K 31/4155; A61K 31/4164; A61K 31/4166; A61K 31/4192; A61K 31/4439; A61K 31/45; A61K 31/496; A61K 31/573; A61K 31/58; A61K 38/09; A61P 33/00; C07K 16/2818; C07K 16/2827

(57) ABSTRACT

A method for treating prostate cancer by administering a first dose of a polyamine inhibitor for a first interval at a beginning of a first treatment cycle; a second dose of the polyamine inhibitor in combination with a dose of an androgen, or a derivative thereof, at a second interval during the first treatment cycle; and a dose of an antiandrogen at a third interval during the first treatment cycle is disclosed.

### CROSS-REFERENCE TO RELATED APPLICATIONS

This application claims the benefit of U.S. Provisional Application No. 63/501,323, filed May 10, 2023, and U.S. Provisional Application No. 63/635,897, filed Apr. 18, 2024, each of which is incorporated herein by reference in its entirety.

### FEDERALLY SPONSORED RESEARCH OR DEVELOPMENT

This invention was made with government support under grants CA204345, CA235863 and CA273167, awarded by the National Institutes of Health, and grants W81XWH-20-1-0079 and HT9425-23-1-0107, awarded by the U.S. Army Medical Research and Material Command. The government has certain rights in the invention.

### BACKGROUND

Metastatic castration-resistant prostate cancer (mCRPC) kills more than 300,000 men worldwide per year. Treatment of CRPC is challenging, however, due to resistance mechanisms that drive persistent androgen receptor (AR) signaling and highly stringent immune tolerance. Prostate cancer (PCa) adaptively regulates AR activity to a level that is optimal for growth and survival. Acute AR inhibition via androgen deprivation therapy produces a “hormonal shock” that leads to the rapid death of AR-expressing PCa cells. Clinical manifestations of this hormonal shock are rapid improvement in pain, rapid decline in prostate-specific antigen (PSA) levels and tumor regression. Unfortunately, androgen deprivation is not curative because a subset of PCa cells survive the hormonal shock. These cells can remain dormant for prolonged periods of time but eventually grow, consistent with the development of castration-resistance. Thus, there is an urgent need for improved therapies for treating PCa, including mCRPC.

### SUMMARY

In some aspects, the presently disclosed subject matter provides a method for treating prostate cancer in a subject in need of treatment thereof, the method comprising administering to the subject: (a) a first dose of a polyamine inhibitor for a first interval at a beginning of a first treatment cycle; (b) a second dose of the polyamine inhibitor in combination with a dose of an androgen, or a derivative thereof, at a second interval during the first treatment cycle; and (c) a dose of an antiandrogen at a third interval during the first treatment cycle.

In certain aspects, the first dose of the polyamine inhibitor is administered for a first interval of about 7±3 days at the beginning of the first treatment cycle.

In certain aspects, the second dose of the polyamine inhibitor in combination with a dose of the androgen, or a derivative thereof, is administered for a second interval of about 56±7 days at a completion of the first interval during the first treatment cycle.

In certain aspects, the second dose of the polyamine inhibitor in combination with a dose of the androgen, or a derivative thereof, is administered for a second interval comprising continuous administration until evidence of prostate cancer disease progression is observed, as evidenced by one or more criteria selected from a rise in serum PSA levels, a worsening of clinical symptoms due to prostate cancer, or worsening disease on imaging studies. In particular aspects, the imaging studies are selected from a computed tomography (CT) scan, magnetic resonance imaging (MRI), a bone scan, PSMA-based positron emission tomography (PET) imaging, and a ¹⁸F-fluciclovine PET/CT scan.

In certain aspects, the dose of an antiandrogen is administered at a third interval of about 56±7 days at a completion of the second interval during the first treatment cycle.

In certain aspects, the dose of an antiandrogen is administered at a completion of the second interval during the first treatment cycle at a third interval comprising continuous administration until evidence of prostate cancer disease progression is observed, as evidenced by one or more criteria selected from a rise in serum PSA levels, a worsening clinical symptoms due to prostate cancer, and worsening disease on imaging studies. In particular aspects, the imaging studies are selected from a CT scan, an MRI, a bone scan, a PSMA-based PET imaging, and a ¹⁸F-fluciclovine PET/CT scan.

In certain aspects, the method further comprises discontinuing administration of the second dose of the polyamine inhibitor in combination with the dose of an androgen, or a derivative thereof, at a completion of the second interval during the first treatment cycle.

In certain aspects, the method further comprises discontinuing administration of the dose of the antiandrogen at a completion of the third interval during the first treatment cycle.

In certain aspects, the first dose and the second dose of the polyamine inhibitor are each about 1000 mg/day in both the first interval and the second interval of the first treatment cycle.

In certain aspects, the dose of the androgen, e.g., testosterone, or an androgen derivative thereof, comprises a supraphysiologic level of androgen (SPA), or the androgen derivative thereof. In particular aspects, the supraphysiological level of androgen (SPA) comprises a serum concentration of androgen, e.g., testosterone, between about 3 to about 10 times a normal serum concentration of androgen, e.g., testosterone, of the subject. In more particular aspects, androgen, e.g., testosterone, or an androgen derivative thereof, is given in sufficient quantity to produce a supraphysiologic level of androgen (SPA) comprising a serum concentration of androgen, e.g., testosterone of, greater than about 1,500 ng/dL or a concentration of an androgen derivative that is equivalent to a level of an androgen, e.g., testosterone, of greater than about 1500 ng/dL. In yet more particular aspects, the dose of the androgen, e.g., testosterone, is between about 400 mg to about 500 mg over a 28-day treatment cycle when administered via intramuscular injection or between about 15 to about 20 mg per day when given by a method other than IM. In particular aspects, the administration method other than IM is selected from transdermal, buccal, and intranasal administration.

In certain aspects, the dose of the antiandrogen is between about 50 mg/day to about 1200 mg/day.

In other aspects, the method further comprises administering one or more androgen synthesis inhibitors. In certain aspects, the one or more androgen synthesis inhibitors are selected from the group consisting of a CYP17A1 inhibitor, a CYP11A1 (P450scc) inhibitor, a 5α-Reductase inhibitor, and combinations thereof. In particular aspects, the one or more androgen synthesis inhibitors are selected from the group consisting of abiraterone acetate, ketoconazole, seviteronel, aminoglutethimide, alfatradiol, dutasteride, epristeride, finasteride, and combinations thereof.

In certain aspects, the polyamine inhibitor is selected from the group consisting of a polyamine synthesis inhibitor, a polyamine analog, a polyamine uptake inhibitor and combinations thereof. In particular aspects, the polyamine inhibitor can be administered alone or in complex in a carrier, such as a liposome or nanoparticle, and administered intravenously, subcutaneously, or orally. In more particular aspects, the polyamine inhibitor is selected from an ornithine decarboxylase inhibitor, e.g., difluoromethylornithine (DFMO), or a polyamine analog, e.g., SBP101 (Ivospemin). In yet more particular aspects, the polyamine inhibitor is the ornithine decarboxylase inhibitor difluoromethylornithine (DFMO).

In certain aspects, the androgen, or a derivative thereof, comprises an ester of testosterone or an ester of dihydrotestosterone. In particular aspects, the ester of testosterone or the ester of dihydrotestosterone is selected from a cypionate, enanthate, propionate, butyrate, and undecanoate ester of testosterone or dihydrotestosterone. In more particular aspects, the ester of testosterone is testosterone cypionate or testosterone enanthate. In yet more particular aspects, the ester of testosterone comprises testosterone cypionate.

In certain aspects, the method comprises administering the androgen, or a derivative thereof, orally, transdermally, transbuccally, intranasally, or by intramuscular injection.

In certain aspects, the antiandrogen is selected from the group consisting of bicalutamide, flutamide, nilutamide, apalutamide, darolutamide, enzalutamide, cyproterone acetate, proxalutamide, cimetidine, and topilutamide. In particular aspects, the antiandrogen is selected from the group consisting of enzalutamide, apalutamide, darolutamide, and combinations thereof. In more particular aspects, the antiandrogen comprises enzalutamide.

In certain aspects, the method comprises administering agents that are ligand-independent inhibitors of the function of the androgen receptor. In particular aspects, the ligand-independent inhibitors are selected from the group consisting of N-terminal domain-targeted androgen receptor inhibitors, DNA-binding domain-targeted androgen receptor inhibitors, androgen receptor mRNA inhibitors, and agents that produce degradation of the androgen receptor protein. In more particular aspects, the androgen receptor inhibitor comprises a proteolysis targeting chimera (PROTAC) that can degrade the androgen receptor protein.

In certain aspects, the treatment inhibits growth of castration resistant prostate cancer cells. In certain aspects, the treatment blocks production of one or more polyamines. In particular aspects, the one or more polyamines are selected from putrescine, spermidine, and spermine. In particular aspects, the blocking of the production of one or more polyamines includes blocking an ornithine decarboxylase (ODC) enzyme with DFMO.

In certain aspects, the treatment decreases expression of an oncogene MYC. In certain aspects, the treatment augments an antitumor immune response.

In certain aspects, the method further comprises concurrently administering an androgen deprivation therapy (ADT) to the subject. In particular aspects, the ADT comprises surgical castration or administering a luteinizing hormone-releasing hormone (LHRH) agonist or a LHRH antagonist to the subject. In particular aspects, the LHRH agonist is selected from the group consisting of leuprolide, goserelin, triptorelin, and histrelin. In particular aspects, the LHRH antagonist is selected from the group consisting of degarelix and relugolix.

In other aspects, the method further comprises administering immune checkpoint blockade therapy to the subject if the subject exhibits clinical and/or radiographic progression. In particular aspects, the immune checkpoint blockade therapy comprises administering an anti-PD1/PDL1 antibody or an anti-CTLA4 antibody. In more particular aspects, the anti-PD1/PDL1 antibody is selected from the group consisting of pembrolizumab, nivolumab, and atezolizumab. In more particular aspects, the anti-CTLA4 antibody comprises ipilimumab.

In certain aspects, the subject has progressive prostate cancer after treatment with abiraterone or an antiandrogen in combination with androgen deprivation therapy (ADT) as an initial therapy or as a second-line therapy after development of resistance to primary ADT.

In particular aspects, the prostate cancer comprises castration resistant metastatic prostate cancer. In certain aspects, the subject is asymptomatic. In other aspects, the subject is symptomatic.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copics of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A, FIG. 1B, and FIG. 1C show: FIG. 1A, PSA response to BAT in the TRANSFORMER trial. FIG. 1B, Overall survival to BAT-Enza versus Enza in the TRANSFORMER trial. FIG. 1C, SKCaP-1R PDX tumor growth in NSG mice following no treatment (control), testosterone cypionate sq pellet (continuous SPA), and alternating SPA with enzalutamide (SPA-ENZA);

FIG. 2A, FIG. 2B, FIG. 2C, FIG. 2D, FIG. 2E, FIG. 2F, FIG. 2G, and FIG. 2H show: FIG. 2A, Change in metabolite abundance following 2 weeks of SPA treatment of SKCaP-1R tumors in castrated NSG mice. FIG. 2B, Putrescine abundance in lysed cells (intracellular) or media (extracellular) of LNCaP cells treated with vehicle (VEH) or R1881 10 nM (SPA). FIG. 2C, Putrescine abundance in concentrated plasma of pts with CRPC treated with BAT on the COMBAT trial (NCT03554317). FIG. 2D, ODC and SSAT activity of LNCaP cells treated with VEH or R1881 10 nM (SPA) for 24 hours. FIG. 2E, ODC protein expression by western blot of LNCaP, VCaP, and 22RV1 cells treated with R1881 10 nM (SPA) for 0, 6, 12, 24, and 48 hours. Vinculin was used as a loading control. FIG. 2F, Polyamine synthesis pathway. FIG. 2G, Change in mRNA expression following 22 weeks of ADT in 7 patients with advanced PCa in the Rajan et al dataset. FIG. 2H, Correlation of ODC1 expression with AR score in the SU2C/PCF mCRPC dataset downloaded from cbioportal;

FIG. 3A, FIG. 3B, and FIG. 3C demonstrate that ODC inhibition enhances growth suppression of PCa models by SPA. FIG. 3A. Number of colonies counted at 14 days of LNCaP cells treated with vehicle control (VEH; EtOH 0.1%) or androgen (R1881 0.5 nM) in combination with indicated increasing doses of DFMO with or without putrescine for 96 hours. ** indicates P<0.01. P values by unpaired 2-tailed t test. Ns, not significant. FIG. 3B. Number of colonies counted at 14 days of VCaP cells treated with vehicle control (VEH; EtOH 0.1%) or androgen (R1881 0.5 nM) in combination with indicated increasing doses of DFMO with or without putrescine for 96 hours. * indicates P<0.05, ** indicates P<0.01. P values by unpaired 2-tailed t test. Ns, not significant. FIG. 3C. Tumor size over time of castration-resistant MycCaP (MycCaP-CR) tumors growing subcutaneously in the flank of castrated C57Bl/6 mice treated with empty pellet (Control), supraphysiological androgen (SPA; testosterone cypionate subcutaneous pellet), DFMO (2% in drinking water), or SPA&DFMO. * indicates P<0.05, ** indicates P<0.01, *** indicates P<0.001. P values by ANOVA;

FIG. 4 is a representative trial schema;

FIG. 5A, FIG. 5B, FIG. 5C, FIG. 5D, and FIG. 5E show: FIG. 5A, MYC expression by IHC in a metastatic tumor biopsy from a patient before and after 3 months of BAT. FIG. 5B, Quantification of MYC protein expression from metastatic tumor biopsies from patients before and after 3 months of BAT color-coded by response (non-responder NR; responder R). FIG. 5C, MYC expression by western blot of LNCaP cells treated with combinations of SPA, DFMO, and putrescine. Vinculin was used as a loading control. FIG. 5D, Principal component analysis of RNA and ATAC sequencing of LNCaP cells treated for 5 days with R1881 10 nM (SPA). FIG. 5E, Total and hypusinated EIF5A expression by western blot of LNCaP cells treated with combinations of SPA, DFMO, and putrescine. Vinculin was used as a loading control;

FIG. 6A, FIG. 6B, FIG. 6C, FIG. 6D, FIG. 6E, and FIG. 6F show: FIG. 6A, UMAP plot of 15 T cell markers assessed by flow cytometry of intratumoral T cells derived from castrated FVB mice bearing MYC-CaP-CR tumors treated with empty pellet (control), 2% DEMO in drinking water, testosterone cypionate SQ pellet (SPA), and DEMO+SPA. FIG. 6B, Heatmap projection of selected markers driving UMAP clustering in (FIG. 6A). FIG. 6C, CD8 tumor-infiltrating T cell expression of granzyme and perforin. FIG. 6D, UMAP plot of 12 myeloid cell markers assessed by flow cytometry of intratumoral myeloid cells. FIG. 6E, Heatmap projection of selected markers driving UMAP clustering in (FIG. 6D). FIG. 6F, Tumor-infiltrating macrophage expression of CD206, MHCII, and CD86. Comparison by unpaired two-tailed T test with **p<0.01 and ****p<0.0001;

FIG. 7 shows GSEA of metabolism-associated gene sets comparing responding (R) and non-responding (NR) patients pre-BAT and on C4D1 of BAT;

FIG. 8 shows leading-edge genes driving differential enrichment of HALLMARK_OXIDATIVE_PHOSPHORYLATION in NR compared with R on C4D1 of BAT′;

FIG. 9 demonstrates that LNCaP and VCaP are SPA-sensitive prostate cancer cell lines, while LAPC4 and 22Rv1 are SPA-resistant cell lines. LNCaP, VCaP, LAPC4, and 22Rv1 viable cell number following treatment with VEH or SPA;

FIG. 10 shows global metabolomics of LNCaP cells treated with R1881 10 nM or vehicle control for 5 days;

FIG. 11 shows global metabolomics of LNCaP cells treated with R1881 10 nM or vehicle control for 26 days;

FIG. 12 shows global metabolomics of SKCaP patient-derived xenograft untreated or treated with testosterone cypionate pellet for 2 weeks;

FIG. 13A and FIG. 13B show extracellular flux analysis of prostate cancer cell lines treated with R1881 10 nM for 4 days. (FIG. 13A) Mitochondrial oxygen consumption rate. (FIG. 13B) Extracellular acidification rate;

FIG. 14 shows U-C13glucose tracing of intracellular metabolites of LNCaP cells treated with vehicle control or R1881 10 nM for 4 hours;

FIG. 15 shows U-C13glucose tracing of extracellular metabolites of LNCaP cells treated with vehicle control or R1881 10 nM for 4 hours;

FIG. 16 shows mitotracker green and mitoSOX red staining of LNCaP cells after 4 days treatment with vehicle control or HAD;

FIG. 17 shows global lipidomics of LNCaP cells treated for 5 days or 26 days with HDA, expressed as fold change relative to vehicle control-treated cells;

FIG. 18A, FIG. 18B, FIG. 18C, and FIG. 18D show clinical data from the RESTORE Study. (FIG. 18A) Trial design; (FIG. 18B) Response parameters for patients progressing on Enza (Cohort A) or Abi (Cohort B). PSAso to re-challenge post-BAT with Enza was 71% vs 21% for Abi. (FIG. 18C) Adverse Events due to BAT in at least 10% of patients. (FIG. 18D) Results of QoL surveys comparing 12 wks of BAT vs. baseline on ADT alone;

FIG. 19A, FIG. 19B, FIG. 19C, FIG. 19D, and FIG. 19E show clinical data from the TRANSFORMER Study. (FIG. 19A) Trial design; (FIG. 19B) Response parameters for BAT (Arm A) vs. Enza (Arm B) and results from patients who crossed over to opposite treatment. (FIG. 19C, FIG. 19D) Waterfall plots of PSA response showing initial PSA₅₀ of 26.4% for BAT and 25.5% for Enza and 72.7% in patients crossing from BAT to Enza and 22.2 for patients crossing from Enza to BAT. (FIG. 19E) Kaplan-Meier curve of sum of PSA progression in each stage of trial. (PSA PFS2) demonstrates 2-fold increase in survival for BAT-Enza (28.2 months) vs. Enza-BAT (14.2 months) (HR=0.45, p=0.03);

FIG. 20A, FIG. 20B, FIG. 20C, FIG. 20D, FIG. 20E, FIG. 20F, and FIG. 20G demonstrate that supraphysiological androgen increases de novo polyamine synthesis in prostate cancer models. FIG. 20A. Tumor size over time of SKCaP-1R patient-derived xenograft tumors growing subcutaneously in the flank of castrated NSG mice untreated (Control) or treated with supraphysiological androgen (SPA; testosterone cypionate subcutaneous pellet). Mice were sacrificed and tumors harvested for global metabolomics following 14 days of treatment. FIG. 20B. Principal component analysis with 95% confidence ellipses of metabolite abundance assessed by capillary electrophoresis mass spectrometry in tumors from (FIG. 20A). FIG. 20C. Volcano plot displaying change in metabolite abundance in SKCaP-1R tumors treated with SPA versus Control from (FIG. 20A). FIG. 20D. Polyamine synthesis pathway schematic displaying metabolites altered in abundance by SPA in tumors from (FIG. 20A). Blue indicates significantly decreased by SPA (Log₂FC<−1 and −Log₁₀FDR>1.3), red indicates significantly increased by SPA (Log₂FC>1 and −Log₁₀FDR>1.3), black indicates not significantly changed by SPA (−Log₂FC>−1 and <1 or −Log₁₀FDR<1.3). FIG. 20E. Isotope tracing schematic. LNCaP or VCaP cells were treated with either vehicle control (VEH; EtOH 0.1%) or SPA (R1881 10 nM) for 24 hours then incubated in media containing either uniformly-labeled ¹³C-arginine (Silac with 1.1-mM U-¹³C-arginine and 100-μM unlabeled putrescine) or ¹³C-putrescine (RPMI, which contains 1.1 mM unlabeled arginine, with 100 μM U-¹³C-putrescine) in addition to VEH or SPA. Metabolites were extracted from cells at 24 hours and abundance of unlabeled and labeled indicated metabolites determined by liquid-chromatography mass spectrometry. FIG. 20F. Contribution of polyamine synthesis pathway metabolites from arginine in LNCaP and VCaP cells treated with VEH or SPA as per experimental design of (FIG. 20E). P values by unpaired 2-tailed t test. FIG. 20G. Contribution of polyamine synthesis pathway metabolites from putrescine in LNCaP and VCaP cells treated with VEH or SPA as per experimental design of (FIG. 20E). P values by unpaired 2-tailed t test;

FIG. 21A and FIG. 21B demonstrate that SPA increases intracellular and extracellular polyamines in prostate cancer cell lines. FIG. 21A. Relative abundance of indicated metabolites measured by LC-MS in LNCaP and VCaP cell lysates following 48 hours of treatment with vehicle control (VEH; EtOH 0.1%) or supraphysiological androgen (SPA; R1881 10 nM). P values by unpaired 2-tailed t test. FIG. 21B. Relative abundance of indicated metabolites measured by LC-MS in LNCaP and VCaP media following 48 hours of treatment with VEH or SPA as per (FIG. 21A). P values by unpaired 2-tailed t test;

FIG. 22A, FIG. 22B, FIG. 22C, FIG. 22D, FIG. 22E, FIG. 22F, FIG. 22G, FIG. 22H, FIG. 22I, and FIG. 22J demonstrate that AR regulates expression of polyamine synthesis enzymes. FIG. 22A. Schematic highlighting key enzymes that regulate polyamine synthesis in mammalian cells. FIG. 22B. Protein expression of ODC, AMD1, ARG2, AR, PSA, and MYC by western blot of prostate cancer cell lines treated with increasing durations of supraphysiological androgen (SPA; R1881 10 nM) for 0, 6, 12, 24, and 48 hours. Representative blot of n=3 independent experiments. Vinculin is used as a loading control. FIG. 22C. Protein expression of ODC, AMD1, ARG2, and AR by western blot of LNCaP cells expressing doxycycline-inducible shRNA against AR pre-treated with or without doxycycline (doxy) for 48 hours then treated with vehicle control (V; EtOH 0.1%) or SPA (S) for 24 hours. Representative blot of n=2 independent experiments. Vinculin is used as a loading control. FIG. 22D. Putrescine and spermidine abundance measured by HPLC in media of cells treated as per (FIG. 22C). M is a cell-free media control. P values by unpaired 2-tailed t test. Biological replicates indicated in gray with mean of each independent experiment in color. FIG. 22E. Correlation of ODC1, AMD1, ARG2, SRM, and SMS transcript expression (log₂FPKM) with AR Score in 264 metastatic tumor biopsies of patients with metastatic castration-resistant prostate cancer (mCRPC) in the SU2C/PCF dataset available on cBioPortal. r and p values determined by Pearson's correlation calculation. FIG. 22F. Volcano plot comparing mRNA transcript abundance in tumors of 7 patients with prostate cancer treated for 22 weeks with androgen deprivation therapy (ADT) versus pretreatment samples in the Rajan et al, 2014 dataset. FIG. 22G. Heatmap comparing mRNA transcript abundance of ODC1, AMD1, ARG2, SRM, and SMS across cancer types included in the TCGA Pan-Can dataset available on cBioPortal (n=10,071). Euclidean clustering indicates that prostate adenocarcinoma (PRAD) clusters separately from other cancer types due to its distinctly high expression of these transcripts. FIG. 22H. Abundance of spermine purified from human tissues obtained at autopsy of 69 individuals by Hamalainen, 1941. P values by unpaired 2-tailed t test. Quantification from blood, stomach, bone marrow, and thymus are not presented, as fewer than 5 samples were measured. FIG. 22I. Protein expression of ODC and AR by western blot of human T cells cultured for 24 hours with vehicle control (EtOH 0.1%), SPA, or enzalutamide (ENZA; 10 μM) in the presence of no stimulation or CD3- and CD28-stimulation with dynabeads. Vinculin is used a loading control. FIG. 22J. Quantification of protein expression of ODC by western blot as per (FIG. 22I), displaying data from independent experiments of 5 different donors with indicated sex. P values by unpaired 2-tailed t test;

FIG. 23A and FIG. 23B demonstrate that SPA increases activity of ODC and AMD1 in prostate cancer cell lines. FIG. 23A. ODC activity in cell lysates of LNCaP and VCaP cells treated with vehicle control (VEH; EtOH 0.1%) or supraphysiological androgen (SPA; R1881 10 nM) for 24 hours. P values by unpaired 2-tailed t test. FIG. 23B. AMD1 activity in cell lysates of LNCaP and VCaP cells treated with vehicle control (VEH; EtOH 0.1%) or supraphysiological androgen (SPA; R1881 10 nM) for 24 hours. P values by unpaired 2-tailed t test;

FIG. 24A and FIG. 24B demonstrate that SPA does not alter abundance of hypusinated eIF5A. FIG. 24A. Schematic highlighting generation of hypusinated eIF5A from spermidine. FIG. 24B Protein expression of hypusinated eIF5A (EIF5A^{H}) and total eIF5A in LNCaP, LAPC4, and 22Rv1 cells following treatment with vehicle control (VEH; EtOH 0.1%) or combinations of supraphysiological androgen (SPA; R1881 10 nM), DFMO (5 mM), and putrescine (100 μM) as indicated for 96 hours. Vinculin is used as a loading control. Representative blots of n=2 independent experiments;

FIG. 25A, FIG. 25B, and FIG. 25C show the characteristics of subjects in the Hamalainen autopsy study. FIG. 25A Age of each subject. FIG. 25B. Sex of the subjects. FIG. 25C. Cause of death of the subjects;

FIG. 26A, FIG. 26B, FIG. 26C, FIG. 26D, FIG. 26E, and FIG. 26F demonstrate that MYC antagonizes AR-stimulated expression of ODC and AMD1. FIG. 26A. Protein expression of AMD1, ODC, PSA, and MYC by western blot of LNCaP cells expressing empty vector (EV) or MYC expression vector (MYC) treated with vehicle control (V; EtOH 0.1%) or supraphysiological androgen (S; R1881 10 nM) for 96 hours. Vinculin is used as a loading control. FIG. 26B. Change in RNA expression (Log₂Fold-Change) of MYC, ODC1, AMD1, KLK3, SRM, ARG2, and SMS by RNAseq in LNCaP cells expressing empty vector (EV) or MYC expression vector (MYC) treated with supraphysiological androgen (S; R1881 10 nM) relative to those treated with vehicle control (V; EtOH 0.1%) for 96 hours. P values by unpaired 2-tailed t test. FIG. 26C. Principal component analysis of RNA transcript abundance by RNAseq of LNCaP-EV and LNCaP-MYC cells treated with VEH or SPA as per (FIG. 26B). FIG. 26D. A comparison of change of individual RNA transcript abundance by SPA in LNCaP-EV cells versus LNCaP-MYC cells by 2-dimensional Cartesian coordinate system. The line of best fit (dotted red) has a shallower slope than the line of unity (solid blue) indicating that many transcripts have reduced change by SPA in LNCaP-MYC cells. Quantitatively, while 18% and 14% of transcripts are more increased or decreased, respectively, by SPA in LNCaP-MYC cells, 38% and 30% of transcripts are less increased or decreased, respectively, by SPA in LNCAP-MYC cells. FIG. 26E. Change in RNA expression (Log₂Fold-Change) of MYC, ODC1, AMD1, KLK3, SRM, ARG2, and SMS by RNAseq in VCaP cells with control knock-down or MYC knock-down by siRNA. P values by unpaired 2-tailed t test. Data reanalyzed from Guo et al, 2021. FIG. 26F. Schematic highlighting that the inhibition of MYC by SPA can function as an amplifying circuit to further increase expression of ODC1, AMD1, and KLK3 by SPA;

FIG. 27A, FIG. 27B, FIG. 27C, FIG. 27D, FIG. 27E, FIG. 27F, FIG. 27G, and FIG. 27H demonstrate that inhibition of ODC increases downregulation of MYC by SPA. FIG. 27A. Principal component analysis of RNA transcript abundance by RNAseq of LNCaP cells treated with vehicle control (VEH; EtOH 0.1%), supraphysiological androgen (SPA, R1881 10 nM), DFMO (5 mM), DFMO and putrescine (PUT; 100 μM), DFMO and SPA, and DFMO and SPA and PUT for 96 hours. FIG. 27B. Overlap of differentially expressed transcripts occurring due to treatment as per (FIG. 27A). FIG. 27C. Volcano plot displaying change in transcript abundance in LNCaP cells treated with DFMO and SPA versus VEH as per (FIG. 27A). FIG. 27D. RNA and protein expression of MYC by RT-PCR and western blot of LNCaP and VCaP cells treated with vehicle control (VEH; EtOH 0.1%) or combinations of supraphysiological androgen (SPA, R1881 10 nM), DEMO (5 mM), and putrescine (PUT; 100 μM), as indicated, for 96 hours. MYC RNA expression was normalized to ACTB and MYC protein expression was normalized to vinculin. P values by unpaired 2-tailed t test. FIG. 27E. Volcano plot displaying normalized enrichment scores of HALLMARK gene sets in LNCaP cells treated with DFMO and SPA versus VEH as per (FIG. 27A). FIG. 27F. Change in RNA transcript abundance of genes located with the 8q24 topologically associated domain (TAD) in LNCaP cells treated as per (FIG. 27A). FIG. 27G. Protein expression of MYC, AMD1, P-S6K (T389), total S6K by western blot in LNCaP and VCaP cells treated with vehicle control (VEH, EtOH 0.1%) or combinations of supraphysiological androgen (SPA; R1881 10 nM), DFMO (5 mM), putrescine (PUT; 100 μM), spermidine (SPD; 100 μM), spermine (SPM; 100 μM), toluenesulfonic acid (TSA; 200 μM), S-adenosyl-methionine-toluenesulfonic acid (TSA-SAM; 25 μM, 50 μM, 100 μM, 200 μM), and SAM486 (2 μM), as indicated, for 96 hours. All samples were treated with aminoguanidine (AG; 1 mM). Vinculin is used as a loading control. FIG. 27H. Schematic displaying effects of SPA and DFMO combination treatment. SPA increases expression of ODC and AMD1. DFMO inhibits ODC activity, leading to depletion of downstream polyamines. Decreased spermine reduces negative regulation of AMD1, further increasing its abundance. This marked increased in AMD1 by SPA and DFMO leads to depletion of SAM, which leads to decreased MYC expression;

FIG. 28A and FIG. 28B demonstrate that changes in transcript abundance can be attributed to one or both treatments. FIG. 28A. A comparison of change of individual RNA transcript abundance by DFMO versus SPA&DFMO in LNCaP cells treated as per FIG. 24A. Black line is line of unity. AMD1 is increased to the same degree in both treatments indicating effect in combination treatment is likely due to DFMO. GNMT is only increased in SPA&DFMO indicating effect in combination treatment is likely not due to DFMO. MYC is decreased in both, but to a greater degree in SPA&DFMO, indicating effect in combination treatment is likely partially attributable to DFMO. FIG. 28B. A comparison of change of individual RNA transcript abundance by SPA versus SPA&DFMO in LNCaP cells treated as per FIG. 24A. Black line is line of unity. GNMT is increased to the same degree in both treatments indicating effect in combination treatment is likely due to SPA. AMD1 is increased in both, but to a greater degree in SPA&DFMO, indicating effect in combination treatment may be partially attributable to SPA. MYC is decreased in both, but to a greater degree in SPA&DFMO, indicating effect in combination treatment is partially attributable to SPA;

FIG. 29 demonstrates that DFMO stabilizes AMD1 transcript. Relative expression of AMD1 transcript by RT-PCR in LNCaP cells treated with VEH or DFMO for 96 hours then actinomycin 5 μg/mL for 0, 1, or 8 hours as indicated;

FIG. 30 shows a representative clinical trial study design. Clinical trial study design to test efficacy of combination therapy with DFMO and BAT alternating with enzalutamide for patients with metastatic castration-resistant prostate cancer. Patients will be treated with a 7-day lead-in of DFMO 1000 mg PO BID monotherapy, then 56 days of combination therapy with DFMO and BAT (testosterone cypionate 400 mg IM q28d), then 56 days of enzalutamide 160 mg PO daily. Multiple specimens will be collected for correlative studies;

FIG. 31 shows that androgens have context-dependent effects in prostate cancer;

FIG. 32 shows a representative example of bipolar androgen therapy (BAT), which is the administration of monthly testosterone;

FIG. 33 is a diagram illustrating that BAT induces a persister metabolic program that enables development of acquired resistance;

FIG. 34 demonstrates that supraphysiological androgen (SPA) alters the metabolome of the SKCaP-1R PDX model of mCRPC;

FIG. 35 demonstrates that SPA increases intratumoral polyamines;

FIG. 36 demonstrates that SPA increases intratumoral polyamines by increasing de novo synthesis;

FIG. 37 demonstrates that SPA increases expression and activity of ODC and AMD1, rate-limiting enzymes of polyamine synthesis;

FIG. 38 shows the chemical structure of ornithine and difluoromethylornithine (i.e., DFMO or eflornithine), which is a clinically-utilized irreversible inhibitor of ODC;

FIG. 39 demonstrates that DFMO enhances downregulation of MYC by SPA;

FIG. 40 are schemes representing a hypothesis that BAT&DFMO will have enhanced efficacy compared with historical controls of BAT monotherapy;

FIG. 41 show androgen and polyamine elimination alternating with Xtandi: the APEX study. 50 asymptomatic pts with mCRPC who have progressed on NHA. 20 slots reserved for pts with met amenable to biopsy. No hearing loss that affects every day life or requires hearing aid; and

FIG. 42A, FIG. 42B, and FIG. 42C show representative data for the first three patients of the APEX study.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The presently disclosed subject matter involves the combination of high dose testosterone (i.e., supraphysiologic androgen) and the polyamine synthesis inhibitor difluoromethylornithine (DFMO) as therapy for castrate resistant prostate cancer. Preliminary data show that administration of supraphysiologic levels of androgen (SPA) can inhibit the growth of castration resistant prostate cancer cells. We have termed this Bipolar Androgen Therapy (BAT). We have tested BAT in multiple clinical trials to demonstrate safety and efficacy. We have been actively studying mechanisms underlying the ability of SPA to inhibit prostate cancer cell growth. We have discovered that SPA induces production of high levels of the polyamines putrescine, spermidine, and spermine. We found that blocking production of these polyamines augments the effect of SPA. This polyamine inhibition is achieved by blocking the enzyme ornithine decarboxylase (ODC) with DFMO. The combination of SPA+DFMO inhibits prostate cancer growth in vitro and in vivo. The combination decreases expression of the oncogene MYC. The combination also might have an ability to augment an antitumor immune response.

More particularly, in some embodiments, the presently disclosed subject matter provides a method for treating prostate cancer in a subject in need of treatment thereof, the method comprising administering to the subject: (a) a first dose of a polyamine inhibitor for a first interval at a beginning of a first treatment cycle; (b) a second dose of the polyamine inhibitor in combination with a dose of an androgen, or a derivative thereof, at a second interval during the first treatment cycle; and (c) a dose of an antiandrogen at a third interval during the first treatment cycle.

In certain embodiments, the first dose of the polyamine inhibitor is administered for a first interval of about 7±3 days at the beginning of the first treatment cycle.

In certain embodiments, the second dose of the polyamine inhibitor in combination with a dose of the androgen, or a derivative thereof, is administered for a second interval of about 56±7 days, including 49, 50, 51, 52, 53, 54, 55, 56, 57, 58, 59, 60, 61, 62, and 63 days, at a completion of the first interval during the first treatment cycle.

In certain embodiments, the second dose of the polyamine inhibitor in combination with a dose of the androgen, or a derivative thereof, is administered for a second interval comprises continuous administration until evidence of prostate cancer disease progression is observed, as evidenced by one or more criteria selected from a rise in serum PSA levels, a worsening of clinical symptoms due to prostate cancer, or worsening disease on imaging studies. In particular embodiments, the imaging studies are selected from a CT scan, an MRI, a bone scan, PSMA-based PET imaging, and a ¹⁸F-fluciclovine PET/CT scan. ¹⁸F-fluciclovine, (anti-1-amino-3-¹⁸F-fluorocyclobutane-1-carboxylic acid, or Axumin®), is a non-naturally occurring amino acid PET radiotracer approved by the U.S. FDA for detecting suspected recurrent prostate cancer using PET/CT.

In certain embodiments, the dose of an antiandrogen is administered at a third interval of about 56±7 days, including 49, 50, 51, 52, 53, 54, 55, 56, 57, 58, 59, 60, 61, 62, and 63 days, at a completion of the second interval during the first treatment cycle.

In certain embodiments, the dose of an antiandrogen is administered at a completion of the second interval during the first treatment cycle at a third interval comprising continuous administration until evidence of prostate cancer disease progression is observed, as evidenced by one or more criteria selected from a rise in the serum PSA levels, a worsening of clinical symptoms due to prostate cancer, and worsening disease on imaging studies. In particular embodiments, the imaging studies are selected from a CT scan, an MRI, a bone scan, PSMA-based PET imaging, and a ¹⁸F-fluciclovine PET/CT scan.

In certain embodiments, the method further comprises discontinuing administration of the second dose of the polyamine inhibitor in combination with the dose of an androgen, or a derivative thereof, at a completion of the second interval during the first treatment cycle.

In certain embodiments, the method further comprises discontinuing administration of the dose of the antiandrogen at a completion of the third interval during the first treatment cycle.

In certain embodiments, the first dose and the second dose of the polyamine inhibitor are each about 1000 mg/day in both the first interval and the second interval of the first treatment cycle.

In certain embodiments, the dose of the androgen, e.g., testosterone, or an androgen derivative thereof comprises a supraphysiologic level of the androgen, e.g., testosterone, or the androgen derivative (SPA). In particular embodiments, the supraphysiological level of androgen (SPA) comprises a serum concentration of androgen, e.g., testosterone, between about 3 to about 10 times, including 3, 4, 5, 6, 7, 8, 9, and 10 times, a normal serum concentration of androgen, e.g., testosterone, of the subject. In more particular embodiments, androgen, e.g., testosterone, or an androgen derivative thereof is given in sufficient quantity to produce a supraphysiologic level of androgen (SPA) comprising a serum concentration of testosterone of greater than about 1,500 ng/dL or a concentration of an androgen derivative that is equivalent to a level of testosterone of greater than about 1500 ng/dl. In yet more particular embodiments, the dose of the testosterone is between about 400 mg to about 500 mg over a 28-day treatment cycle when administered via intramuscular injection (IM) or between about 15 mg and 20 mg per day when administered by a method other than IM. In particular embodiments, the method other than IM is selected from transdermal, buccal, and intranasal.

In certain embodiments, the dose of the antiandrogen is between about 100 mg/day to about 200 mg/day, including about 100, 105, 110, 115, 120, 125, 130, 135, 140, 145, 150, 155, 160, 165, 170, 175, 180, 185, 190, 195, and 200 mg/day.

In some embodiments, the method further comprises administering one or more androgen synthesis inhibitors. In certain embodiments, the one or more androgen synthesis inhibitors are selected from the group consisting of a CYP17A1 inhibitor, a CYP11A1 (P450scc) inhibitor, a 5α-Reductase inhibitor, and combinations thereof. In particular embodiments, the one or more androgen synthesis inhibitors are selected from the group consisting of abiraterone acetate, ketoconazole, seviteronel, aminoglutethimide, alfatradiol, dutasteride, epristeride, finasteride, and combinations thereof.

In certain embodiments, the polyamine inhibitor is selected from the group that includes a polyamine synthesis inhibitor, a polyamine analog, a polyamine uptake inhibitor, and combinations thereof. In particular embodiments, the polyamine inhibitor can be administered alone or in complex in a carrier, such as a liposome or nanoparticle, and administered intravenously, subcutaneously, or orally. In more particular embodiments, the polyamine inhibitor is selected from the ornithine decarboxylase inhibitor difluoromethylornithine (DFMO) or the polyamine analog SBP101 (Ivospemin).

Representative polyamine analogues/polyamine analogue nanoparticle (ex. SBP-101/Ivospemin) targeting polyamine metabolism through different mechanisms than DFMO are provided in the following references, each of which is incorporated herein in its entirety:
Casero, R. A., Jr. and Woster, P. M. Recent Advances in the Development of Polyamine Analogues as Antitumor Agents. J Med Chem. 52:4551-4573, 2009. PMCID: PMC2762202;
Nowotarski, S. L., Woster, P. M., and Casero, R. A. Polyamines and cancer: Implications for chemotherapy and chemoprevention. Exp. Rev. Molec. Med., 15: e3, 2013. PMCID: PMC4027058;
Xie, Y., Murray-Stewart, T., Wang, Y., Yu, F., Marton, L. J., Casero, R. A., and Oupicky, D. Self-Immolative polyamine nanoparticles to deliver miR-34 mimic and target polyamine metabolism for combination cancer therapy. J. Controlled Release, 246:110-119, 2017;
Murray-Stewart, T., Ferrari, E., Xie, Y., Yu, F., Marton, L. J., Oupicky, D., and Casero, R. A. Biochemical evaluation of the anticancer potential of the polyamine-based nanocarrier, Nano11047. PLOS ONE. 12 (4): e0175917, 2017;
Murray Stewart, T., Desai, A. A., Fitzgerald, M. L., Marton, L. J., and Casero, R. A. A phase I dose-escalation study of the polyamine analogue PG-11047 in patients with advanced solid tumors. Cancer Chem. Pharm., 85:1089-1096, 2020. PMCID: PMC7337042;
Murray Stewart, T., Von Hoff, D., Fitzgerald, M., Marton, L. J., Roberto Becerra, C. H., Boyd, T. E., Conklin, P. R., Garbo, L. E., Jotte, R. M., Richards, D. A., Smith, D. A., Stephenson, J. J., Vogelzang, N. J., Wu, H. H., and Casero, R. A. A phase Ib multicenter, dose-escalation study of the polyamine analogue PG-11047 in combination with gemcitabine, docetaxel, bevacizumab, erlotinib, cisplatin, 5-fluorouracil, or sunitinib in patients with advanced solid tumors or lymphoma. Cancer Chem. Pharm. 87:135-144, 2021. PMCID: PMC7855804;
Holbert, C. E., Foley, J. R., Murray Stewart, T., and Casero, R. A. Expanded potential of the polyamine analogue SBP-101 (diethyl dihydroxyhomospermine) as a modulator of polyamine metabolism and cancer therapeutic. Molecular Pharmacol., Int. J. Molec. Sci. DOI: 10.3390/ijms23126798, 2022. PMCID: 9224330;
Holbert, C. E., Foley, J. R., Yu, A., Murray Stewart, T., Phanstiel, O., Oupicky, D., and Casero, R. A. Polyamine-based nanostructures share polyamine transport mechanisms with native polyamine and their analogues: significance for polyamine-targeted therapy. Med. Sci. 10:44, 2022. PMCID: PMC9397040.

In yet more particular embodiments, the polyamine inhibitor is the ornithine decarboxylase inhibitor difluoromethylornithine (DFMO):

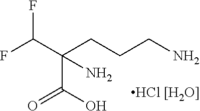 [SMILES: NCCCC(N)(C(=O)O)C(F)F]

Chemical name: 2-(difluoromethyl)-ornithine, monohydrochloride, monohydrate or alpha-difluoromethylornithine (DFMO), or Eflornithine.

In certain embodiments, the androgen, or a derivative thereof, comprises an ester of testosterone or an ester of dihydrotestosterone. In particular embodiments, the ester of testosterone or the ester of dihydrotestosterone is selected from a cypionate, enanthate, propionate, butyrate, and undecanoate ester of testosterone or dihydrotestosterone. In more particular embodiments, the ester of testosterone is testosterone cypionate or testosterone enanthate. In yet more particular embodiments, the ester of testosterone comprises testosterone cypionate:

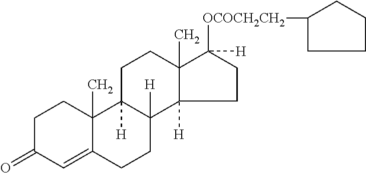

Chemical Name: androst-4-en-3-one, 17-(3-cyclopentyl-1-oxopropoxy)-, (17ß)-

One of ordinary skill in the art would recognize that other androgens, derivatives thereof, including prodrugs, could be used in the presently disclosed methods.

As used herein, the term “androgen” refers to any natural or synthetic steroid hormone that regulates the development and maintenance of male characteristics in vertebrates by binding to androgen receptors. The major androgen in males is testosterone. Other natural androgens include dehydroepiandrosterone (DHEA), which also is referred to as dehydroisoandrosterone or dehydroandrosterone, androstenedione (A4), androstenediol (A5), and dihydrotestosterone (DHT).

Anabolic steroids include natural androgens, such as testosterone, as well as synthetic androgens that are structurally related and have similar effects to testosterone. More particularly, anabolic steroids include testosterone and esters thereof, including, but not limited to, testosterone undecanoate, testosterone enanthate, testosterone cypionate, and testosterone propionate, dihydrotestosterone and esters thereof, including, but not limited to, dihydrotestosterone undecanoate, dihydrotestosterone enanthate, dihydrotestosterone cypionate, and dihydrotestosterone propionate; nandrolone esters, including nandrolone decanoate and nandrolone phenylpropionate; stanozolol; and metandienone (methandrostenolone). Other anabolic steroids include danazol, ethylestrenol, methyltestosterone, norethandrolone, oxandrolone, mesterolone, and oxymetholone, as well as drostanolone propionate (dromostanolone propionate), metenolone (methylandrostenolone) esters, including metenolone acetate and metenolone enanthate, fluoxymesterone, boldenone undecylenate, trenbolone acetate, and esters of DHT. Other anabolic steroids include 1-testosterone (dihydroboldenone), methasterone, trenbolone enanthate, desoxymethyltestosterone, tetrahydrogestrinone, and methylstenbolone.

In some embodiments, the androgen is an ester of testosterone or an ester of another anabolic steroid. Esters of testosterone include, but are not limited to, testosterone caproate, testosterone cypionate, testosterone decanoate, testosterone enanthate, testosterone isobutyrate, testosterone isocaproate, testosterone phenylpropionate, testosterone propionate, testosterone undecanoate, testosterone acetate, testosterone cyclohexylpropionate, testosterone enantate benzilic acid hydrazone, testosterone furoate, testosterone hexahydrobenzoate, testosterone hexahydrobenzylcarbonate, testosterone hexyloxyphenylpropionate, testosterone ketolaurate, testosterone nicotinate, testosterone phenylacetate, testosterone phosphate, testosterone undecylenate, testosterone valerate, testosterone buciclate, polytestosterone phloretin phosphate, testosterone 17β-(1-((5-(aminosulfonyl)-2-pyridinyl)carbonyl)-L-proline) (EC586), testosterone acetate butyrate, testosterone acetate propionate, testosterone benzoate, testosterone butyrate, testosterone diacetate, testosterone dipropionate, testosterone formate, testosterone isovalerate, testosterone palmitate, testosterone phenylbutyrate, testosterone stearate, testosterone sulfate, and dihydrotestosterone esters.

Esters of dihydrotestosterone (DHT; androstanolone, stanolone) include, but are not limited to, androstanolone benzoate, androstanolone enantate, androstanolone propionate, androstanolone valerate, dihydrotestosterone acetate, dihydrotestosterone butyrate, dihydrotestosterone formate, dihydrotestosterone undecanoate, and testifenon (chlorphenacyl DHT ester).

Esters of other natural anabolic steroids include, but are not limited to, androstenediol dipropionate, prasterone enantate, prasterone sulfate, androstenediol 3β-acetate, androstenediol 3β-acetate 17β-benzoate, androstenediol 17β-acetate, androstenediol diacetate, sturamustine,

Esters of synthetic AAS include methandriol esters, including methandriol bisenanthoyl acetate, methandriol dipropionate, methandriol propionate, and methandriol diacetate; nandrolone esters, including nandrolone decanoate, nandrolone phenylpropionate, nandrolone caproate, nandrolone cyclohexanecarboxylate, nandrolone cyclohexylpropionate, nandrolone cypionate, nandrolone furylpropionate, nandrolone hexyloxyphenylpropionate, nandrolone hydrogen succinate, nandrolone laurate, nandrolone propionate, nandrolone sulfate, nandrolone undecanoate, nandrolone 17β-adamantoate, nandrolone acetate, nandrolone benzoate, nandrolone cyclotate, nandrolone enanthate, nandrolone formate, and LS-1727; trenbolone ester, including trenbolone acetate, trenbolone hexahydrobenzylcarbonate, trenbolone enantate, trenbolone undecanoate

Esters of other synthetic AAS include bolandiol dipropionate, bolazine capronate, boldenone acetate, boldenone cypionate, boldenone propionate, boldenone undecylenate (boldenone undecenoate), clostebol acetate, clostebol caproate, clostebol propionate, drostanolone propionate, metenolone acetate, metenolone enantate, norclostebol acetate, oxabolone cypionate, propetandrol (norethandrolone 3β-propionate), stenbolone acetate, 11β-Methyl-19-nortestosterone dodecylcarbonate, dimethandrolone buciclate, dimethandrolone dodecylcarbonate, dimethandrolone undecanoate, mesterolone cypionate, nisterime acetate, trestolone acetate, and trestolone enantate.

In addition to esters, the presently disclosed methods can include ethers of androgens. Ethers of natural AAS include cloxotestosterone acetate, cloxotestosterone, and silandrone. Ethers of synthetic AAS include mepitiostane, methyltestosterone 3-hexyl ether, penmesterol, quinbolone, mesabolone, methoxydienone (methoxygonadiene), and prostanozol.

Androgens can include derivatives of testosterone and other natural or synthetic androgens, including ester and ether prodrugs, and prohormones. Testosterone derivatives include 4-hydroxytestosterone, 11-ketotestosterone, Δ1-testosterone, and 4-Chlorotestosterone. Prohormone-like androgens include 4-androstenediol, 4-dehydroepiandrosterone (4-DHEA), 5-androstenedione, 5-dehydroandrosterone (5-DHA), 11β-hydroxyandrostenedione (11β-OHA4), 11-keto-4-androstenedione, 5-androstenediol, 4-androstenedione, 1-methyl-δ1-4-androstenedione, δ1-4-Androstenedione, dehydroepiandrosterone (DHEA, 5-DHEA), 6-methylidene-δ1-4-androstenedione, 4-hydroxy-4-androstenedione, 10-propargyl-4-androstenedione,

Prodrugs, including ethers, such as cloxotestosterone, quinbolone, and silandrone.

Dihydrotestosterone derivatives, including dihydrotestosterone (DHT), 4,5α-Dihydro-δ1-testosterone, 11-Ketodihydrotestosterone (11-KDHT), 2α-Methyl-4,5α-dihydrotestosterone, 2α,3α-Epithio-3-deketo-4,5α-dihydrotestosterone, 1α-Methyl-4,5α-dihydrotestosterone, 1-Methyl-4,5α-dihydro-δ1-testosterone, 2α-Chloro-4,5α-dihydrotestosterone 3-O-(p-nitrophenyl)oxime, and 2-Methyl-4,5α-dihydro-δ1-testosterone.

Prohormone-like dihydrotestosterone derivatives including 1-androsterone (1-Andro, 1-DHEA), 1-androstenediol (4,5α-dihydro-δ1-4-androstenediol), 1-androstenedione (4,5α-dihydro-δ1-4-androstenedione), 3-deketo-4,5α-dihydro-82-4-androstenedione), and epiandrosterone. Ether prodrugs including mepitiostane (2α,3α-Epithio-3-deketo-4,5α-dihydrotestosterone 17β-(1-methoxycyclopentane)ether), mesabolone (4,5α-Dihydro-δ1-testosterone 17β-(1-methoxycyclohexane)ether), and prostanozol (2′H-5α-Androst-2-eno[3,2-c]pyrazol-17β-ol 17β-tetrahydropyran ether). Azine dimers, including bolazine (3,3-[(1E,2E)-1,2-Hydrazinediylidene]di(2α-methyl-5α-androstan-17β-ol)).

19-Nortestosterone (nandrolone) derivatives including 19-Nortestosterone, 11β-Methyl-19-nortestosterone (11-MNT), dienolone (19-Nor-δ9-testosterone), dimethandrolone (7α,11β-Dimethyl-19-nortestosterone), norclostebol (4-chloro-19-nortestosterone), oxabolone (4-hydroxy-19-nortestosterone, trenbolone (19-Nor-δ9,11-testosterone), and trestolone (MENT) (7α-Methyl-19-nortestosterone). Prohormone-like (nandrolone) derivatives including 7α-Methyl-19-nor-4-androstenedione (MENT dione, trestione), 19-Nor-5-androstenediol, 19-Nor-5-androstenedione, 19-Nordehydroepiandrosterone, bolandiol (nor-4-androstenediol), bolandione (nor-4-androstenedione), dienedione (nor-4,9-androstadienedione), methoxydienone (18-Methyl-19-nor-δ2,5(10)-epiandrosterone 3-methyl ether), and trendione (nor-4,9,11-androstatrienedione). Ester prodrugs including olmantalate (19-Nortestosterone 17β-adamantoate).

17α-Alkylated testosterone derivatives including bolasterone (7α,17α-dimethyltestosterone), calusterone (7,17α-dimethyltestosterone), chlorodehydromethyltestosterone (CDMT) (4-chloro-17α-methyl-δ1-testosterone), enestebol (4-hydroxy-17α-methyl-δ1-testosterone), ethyltestosterone (17α-ethyltestosterone), fluoxymesterone (9α-fluoro-11β-hydroxy-17α-methyltestosterone), formebolone (2-formyl-11α-hydroxy-17α-methyl-δ1-testosterone), hydroxystenozole (17α-methyl-2′H-androsta-2,4-dieno[3,2-c]pyrazol-17β-ol), metandienone (17α-methyl-δ1-testosterone), methylclostebol (4-chloro-17α-methyltestosterone), methyltestosterone (17α-methyltestosterone), oxymesterone (4-Hydroxy-17α-methyltestosterone), and tiomesterone (1α,7α-Diacetylthio-17α-methyltestosterone). Prohormone-like 17α-Alkylated testosterone derivatives including chlorodehydromethylandrostenediol (CDMA) (4-Chloro-17α-methyl-δ1-4-androstenediol), chloromethylandrostenediol (CMA) (4-chloro-17α-methyl-4-androstenediol), methandriol (17α-methyl-5-androstenediol). Ether prodrugs, including methyltestosterone 3-hexyl ether (17α-methyl-4-hydro-δ3,5-testosterone 3-hexyl ether) and penmesterol (17α-Methyl-4-hydro-δ3,5-testosterone 3-cyclopentyl ether).

17α-Alkylated dihydrotestosterone derivatives including androisoxazole (17α-methyl-5α-androstano[3,2-c]isoxazol-17β-ol), desoxymethyltestosterone (3-deketo-17α-methyl-4,5α-dihydro-82-testosterone), furazabol (17α-methyl-5α-androstano[2,3-c][1,2,5]oxadiazol-17β-ol), mestanolone (methyl-DHT) (17α-methyl-4,5α-dihydrotestosterone), methasterone (2α,17α-Dimethyl-4,5α-dihydrotestosterone), methyl-1-testosterone (17α-methyl-4,5α-dihydro-δ1-testosterone), methyldiazinol (3-azi-17α-methyl-4,5α-dihydrotestosterone), methylepitiostanol (2α,3α-epithio-3-deketo-17α-methyl-4,5α-dihydrotestosterone), methylstenbolone (2,17α-dimethyl-4,5α-dihydro-δ1-testosterone), oxandrolone (2-oxa-17-methyl-4,5α-dihydrotestosterone), oxymetholone (2-hydroxymethylene-4,5α-dihydro-17α-methyltestosterone), and stanozolol (17α-methyl-2′H-5α-androst-2-eno[3,2-c]pyrazol-17β-ol. Azine dimers including mebolazine (3,3-[(1E,2E)-1,2-Hydrazinediylidene]di(2α,17α-dimethyl-5α-androstan-17β-ol).

17α-Alkylated 19-nortestosterone derivatives including dimethyltrienolone (7α,17α-Dimethyl-19-nor-δ9,11-testosterone), dimethyldienolone (7α,17α-Dimethyl-19-nor-δ9-testosterone, ethyldienolone (17α-ethyl-19-nor-δ9-testosterone), ethylestrenol (17α-Ethyl-3-deketo-19-nortestosterone), methyldienolone (17α-Methyl-19-nor-δ9-testosterone), methylhydroxynandrolone (MOHN, MHN) (4-Hydroxy-17α-methyl-19-nortestosterone), metribolone (methyltrienolone, R-1881) (17α-methyl-19-nor-δ9,11-testosterone), mibolerone (7α,17α-dimethyl-19-nortestosterone), norboletone (17α-ethyl-18-methyl-19-nortestosterone), norethandrolone (17α-ethyl-19-nortestosterone), normethandrone (17α-methyl-19-nortestosterone), RU-2309 (17α,18-dimethyl-19-nor-δ9,11-testosterone), and tetrahydrogestrinone (THG) (17α-ethyl-18-methyl-19-nor-δ9,11-testosterone). Prohormone-like 17α-alkylated 19-nortestosterone derivatives including bolenol (ethylnorandrostenol) (3-Deketo-17α-ethyl-19-nor-5-androstenediol). Ester prodrugs including propetandrol (17α-Ethyl-19-nortestosterone 3-propionate).

17α-Vinylated testosterone derivatives including vinyltestosterone (17α-ethenyltestosterone). 17α-vinylated 19-nortestosterone derivatives including 17α-ethenyl-19-nortestosterone.

17α-Ethynylated testosterone derivatives including ethisterone (17α-ethynyltestosterone), danazol (2,3-isoxazol-17α-ethynyltestosterone).

17α-Ethynylated 19-nortestosterone derivatives including norethisterone (17α-ethynyl-19-nortestosterone), etynodiol (17α-ethynyl-3-deketo-3β-hydroxy-19-nortestosterone), gestrinone (ethylnorgestrienone, R-2323) (17α-Ethynyl-18-methyl-19-nor-δ9,11-testosterone), levonorgestrel ((−)-norgestrel) ((−)-17α-Ethynyl-18-methyl-19-nortestosterone), lynestrenol (17α-ethynyl-3-deketo-19-nortestosterone), norgestrel (17α-ethynyl-18-methyl-19-nortestosterone, norgestrienone (17α-ethynyl-19-nor-δ9,11-testosterone), and tibolone (7α-methyl-17α-ethynyl-19-nor-δ5(10)-testosterone. Ethers including quingestanol (4-hydro-19-nor-δ3,5-testosterone 3-cyclopentyl ether). Esters including etynodiol diacetate (17α-ethynyl-3-deketo-3β-hydroxy-19-nortestosterone 3β,17β-diacetate), norethisterone acetate (17α-ethynyl-19-nortestosterone 17β-acetate), and norethisterone enanthate (17α-ethynyl-19-nortestosterone 17β-enanthate). Ethers and esters including quingestanol acetate (4-hydro-17α-ethynyl-19-nor-δ3,5-testosterone 3-cyclopentyl ether 17β-acetate).

In certain embodiments, the method comprises administering the androgen, or a derivative thereof, orally, transdermally or by intramuscular injection.

In certain embodiments, the antiandrogen is selected from the group consisting of bicalutamide, flutamide, nilutamide, apalutamide, darolutamide, enzalutamide, cyproterone acetate, proxalutamide, cimetidine, and topilutamide. In particular embodiments, the antiandrogen is selected from the group consisting of enzalutamide, apalutamide, darolutamide, and combinations thereof. In more particular embodiments, the antiandrogen comprises enzalutamide:

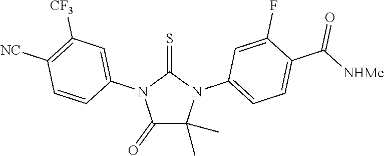 [SMILES: CNC(=O)c1ccc(N2C(=S)N(c3ccc(C#N)c(C(F)(F)F)c3)C(=O)C2(C)C)cc1F]

Chemical Name: 4-{3-[4-cyano-3-(trifluoromethyl)phenyl]-5,5dimethyl-4-oxo-2-sulfanylideneimidazolidin-1-yl}-2-fluoro-N-methylbenzamide.

In certain embodiments, the treatment inhibits growth of castration resistant prostate cancer cells. In certain embodiments, the treatment blocks production of one or more polyamines. In particular embodiments, the one or more polyamines are selected from putrescine, spermidine, and spermine. In particular embodiments, the blocking of the production of one or more polyamines includes blocking an ornithine decarboxylase (ODC) enzyme with DFMO.

In certain embodiments, the treatment decreases expression of an oncogene MYC. In certain embodiments, the treatment augments an antitumor immune response.

In certain embodiments, the method further comprises concurrently administering an androgen deprivation therapy (ADT) to the subject. In particular embodiments, the ADT comprises surgical castration or administering a luteinizing hormone-releasing hormone (LHRH) agonist or a LHRH antagonist to the subject. In certain embodiments, the LHRH agonist is selected from the group consisting of leuprolide (Lupron, Eligard), goserelin (Zoladex), triptorelin (Trelstar), and histrelin (Vantas). In certain embodiments, the LHRH antagonist is selected from the group consisting of Degarelix (Firmagon) and Relugolix (Orgovyx).

In other embodiments, the method further comprises administering immune checkpoint blockade therapy to the subject if the subject exhibits clinical and/or radiographic progression. In particular embodiments, the immune checkpoint blockade therapy comprises administering an anti-PD1/PDL1 antibody or an anti-CTLA4 antibody. In more particular embodiments, the anti-PD1/PDL1 antibody is selected from the group consisting of pembrolizumab, nivolumab, and atezolizumab. In more particular embodiments, the anti-CTLA4 antibody comprises ipilimumab.

In certain embodiments, the subject has progressive prostate cancer after treatment with abiraterone in combination with androgen deprivation therapy (ADT) as an initial therapy or as a second-line therapy after development of resistance to primary ADT.

In particular embodiments, the prostate cancer comprises castration resistant metastatic prostate cancer. In certain embodiments, the subject is asymptomatic. In other embodiments, the subject is symptomatic.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed composition of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

In general, the “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; poultry, such as domestic fowls including, but not limited to chickens, turkeys, geese, ducks, quail, guinea fowl, and pigeons; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

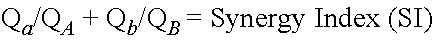

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In another embodiments, the present disclosure provides a pharmaceutical composition including one compound described herein alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the presently disclosed compounds are administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick.

As used herein, the term administrating via an “intranasal route” refers to administering by way of the nasal structures. It has been found that the presently disclosed compounds are much more effective at penetrating the brain when administered intranasally.

Intranasal administration generally allows the active agent to bypass first pass metabolism, thereby enhancing the bioavailability of the active agent. Such delivery can offer several advantages over other modes of drug delivery, including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Nasal administration of a drug also can facilitate compliance, especially for pediatric patients, geriatric patients, patients suffering from a neurodegenerative disease, or other patients for which swallowing is difficult, e.g., patients suffering from nausea, such as patients undergoing chemotherapy, or patients with a swallowing disorder.

Intranasal (“i.n.” or “IN”) delivery of an agent to a subject can facilitate delivery of the agent to the brain and/or peripheral nervous system. Such administration is non-invasive and offers several advantages including avoidance of hepatic first pass clearance, rapid onset of action, frequent self-administration and easy dose adjustments. Small molecules have an added advantage of being absorbed paracellularly through the nasal epithelium after which, these molecules can then directly enter the CNS through the olfactory or the trigeminal nerve associated pathway and can be directly transported to the brain upon intranasal administration.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol monocaprate (Capmul™), mixtures of mono-, di-, and triglycerides and mono- and di-fatty esters of PEG (Labrafil™), palmitate, glycerol monostearate, and phospholipids can be used to administer the presently disclosed compounds intranasally.

The presently disclosed compounds also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed compounds include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug. Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.).

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

In some embodiments, the presently disclosed subject matter provides a kit comprising one or more of a polyamine inhibitor, an androgen, or a derivative thereof, an antiandrogen, one or more reagents, and instructions for use.

In certain embodiments, the disclosed kits comprise one or more containers, including, but not limited to a vial, tube, ampule, bottle and the like, for containing pharmaceutical compositions of one or more of a polyamine inhibitor, an androgen, or a derivative thereof, and an antiandrogen. The compositions may be solvated, in suspension, or powder form, and may then be reconstituted in the pharmaceutically acceptable carrier to provide the pharmaceutical composition. The one or more containers also can be carried within a suitable carrier, such as a box, carton, tube or the like. Such containers can be made of plastic, glass, laminated paper, metal foil, or other materials suitable for holding medicaments.

In certain embodiments, the container can hold a pharmaceutical composition and may have a sterile access port (for example the container may be an intravenous solution bag or a vial having a stopper pierceable by a hypodermic injection needle).

Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Testosterone and Difluoromethylornithine Combination Therapy for Treating Prostate Cancer

### 1.1 Overview

This Example includes the development of a novel therapy for advanced PCa. This therapy will utilize combined supraphysiological androgen (SPA) with difluoromethylornithine (DFMO) alternating every 2 months with the anti-androgen enzalutamide. The innovative and novel aspects of this Example include, but are not limited to the following.

### 1.1.1 Repeat Hormone Shock Therapy

Inhibition of signaling through the androgen receptor (AR) is arguably the most effective systemic therapy targeting a single pathway for any cancer type. Acute ablation of AR signaling results in cell death, stress, and dysfunction of PCa. Adaptation to this initial shock occurs over time, however, resulting in castration-resistance and ultimately culminating in patient mortality. We have pioneered the concept that castration-resistance confers sensitivity to a reverse hormone shock by exposure to SPA, which now overwhelms CRPC by a multitude of mechanisms. This Example presents repeat hormone shock therapy by shifting the hormone microenvironment of PCa every 2 months between polar extremes of exposure to SPA and the antiandrogen enzalutamide for repeat tumor insult. This research is a substantive departure from the status quo in that it will incorporate SPA, the antithesis of standard-of-care AR inhibition, into treatment for patients with advanced PCa.

### 1.1.2 Adaptive Therapy Design

Currently, all treatments for metastatic PCa are administered continuously until progression. This persistent therapeutic pressure selects for PCa resistance, rendering all available treatments useful for a limited duration of time. In contrast, this Example presents an adaptive therapy design of alternating treatment every 2 months to minimize acquired resistance mechanisms and maximize utility of hormone shock therapy.

### 1.1.3 Targeting PCa Metabolic Adaptation to Cancer Therapy

An emerging idea is that cancer therapy resistance can be driven by metabolic plasticity in addition or in lieu of genomic and gene expression changes. This Example provides preliminary evidence that enhanced de novo polyamine synthesis contributes to resistance to SPA, which has not been previously reported. This contribution may occur by stimulating MYC expression, facilitating transcriptional adaptations to stress by altering epigenetic regulation, and stimulating adaptive protein synthesis through the elongation factor eIF5A. We propose to inhibit this pathway with DFMO, renewing the idea that cancer cell metabolism may represent an important drug target in PCa.

### 1.1.4 Overcoming PCa Immune Tolerance

Cancer cell metabolic programs shape the tumor microenvironment and influence function of surrounding immune and stromal cells. Prostate cancer is classically an immunologically “cold” tumor that fails to respond to T cell checkpoint inhibitors. Without wishing to be bound to any one particular theory, it is thought that this immunosuppressive microenvironment is driven in part through AR-mediated stimulation of polyamine production and secretion, which is exacerbated by SPA. We aim to reverse this immunosuppression by limiting polyamine production through the use of DFMO.

### 1.2 Scope

Metastatic castration-resistant prostate cancer (mCRPC) kills >300,000 men worldwide per year. Thus, there is an urgent need for improved therapies. Treatment of CRPC is challenging due to resistance mechanisms that drive persistent androgen receptor (AR) signaling and highly stringent immune tolerance. This Example presents an innovative treatment strategy designed to overcome these therapeutic barriers.

Prostate cancer (PCa) adaptively regulates AR activity to a level that is optimal for growth and survival. Acute AR inhibition via androgen deprivation therapy produces a “hormonal shock” that leads to the rapid death of AR-expressing PCa cells. Clinical manifestations of this hormonal shock are rapid improvement in pain, rapid decline in PSA levels and tumor regression. Unfortunately, androgen deprivation is not curative because a subset of PCa cells survive the hormonal shock. These cells can remain dormant for prolonged periods of time but eventually grow, consistent with the development of castration-resistance.

A major driver of castration resistance is the adaptive upregulation of AR activity, often through AR protein overexpression, to a sufficient level to allow for renewed growth. This marked overexpression of AR makes PCa cells once again vulnerable to hormone shock, achieved not through further androgen blockade, but through exposure to supraphysiologic levels of androgen (SPA). Based on extensive preclinical studies, we developed Bipolar Androgen Therapy (BAT), which involves cyclic administration of SPA as treatment for CRPC. We have completed four clinical studies in approximately 350 patients and demonstrated that BAT is safe, produces PSA/objective response in 20-30% of men, and improves quality of life. We have demonstrated that resistance to SPA is primarily due to adaptive downregulation of AR.

AR downregulation makes PCa once again sensitive to hormone shock produced by re-exposure to antiandrogens. These combined results have established sequential hormone shock achieved through alternating SPA and antiandrogen as a potential new treatment paradigm for CRPC. Here we propose to build on these results with a goal to improve the response to SPA. Our preliminary data indicate that SPA enhances CRPC expression of ornithine decarboxylase (ODC), leading to a marked increase in intracellular and excreted polyamines in PCa models and patients. These polyamines drive PCa persistence despite SPA, as the highly specific and clinically-utilized inhibitor of ODC, difluoromethylornithine (DFMO), markedly enhanced SPA-induced growth inhibition of CRPC models.

Accordingly, this Example includes polyamine inhibitors, such as DFMO, as cancer therapies by adding DFMO to the treatment backbone of alternating SPA and enzalutamide. This treatment protocol is designed to enhance the effectiveness of SPA through combination with DFMO while disrupting adaptive regulation of AR expression through alternating hormone shock thereby minimizing resistance to both SPA and enzalutamide. Without wishing to be bound to any one particular theory, it is thought that DFMO in combination with SPA will lead to enhanced CRPC tumor regression through (1) cancer-cell intrinsic mechanisms including dual suppression of MYC and impaired adaptive epigenetic reprogramming and (2) a cancer cell-extrinsic mechanism via enhancement of a tumor immune response. To test this hypothesis, we propose the following.

1.2.1 Conduct a Biomarker-Rich Phase II Trial of Sequential Repeat Cycles of SPA+DFMO Followed by Enzalutamide in Patients with mCRPC

In representative embodiments, approximately 50 patients with mCRPC will receive repeat cycles of SPA+DFMO for 2 months followed by enzalutamide for 2 months to assess whether this treatment regimen will improve the PSA response rate compared to SPA monotherapy historical controls. Proposed correlative studies will be conducted using metastatic biopsies obtained prior to treatment and after 2 months of SPA+DFMO.

### 1.2.2 Assess the Ability of DFMO to Reduce Polyamine Production and Abundance Leading to Enhanced Downregulation of MYC and Epigenetic Reprogramming in the Presence of SPA

We will confirm that DFMO inhibits ODC to prohibit the spike in polyamines induced by SPA monotherapy. We will assess the cancer cell-intrinsic growth-inhibitory effect of combination therapy by assessing MYC, activating and repressive histone modifications, and the global metabolome and transcriptome of metastatic biopsies by single cell analyses.

### 1.2.3 Evaluate the Ability of DFMO to Augment a Tumor Immune Response in the Presence of SPA

We will assess the cancer cell-extrinsic growth-inhibitory effect of combination therapy by assessing the abundance, phenotype, and function of intratumoral and circulating immune cells by single cell analyses.

### 1.3 Background

1.3.1 BAT is a Promising Therapy, and Alternating BAT with AR Inhibition May Provide the Greatest Benefit

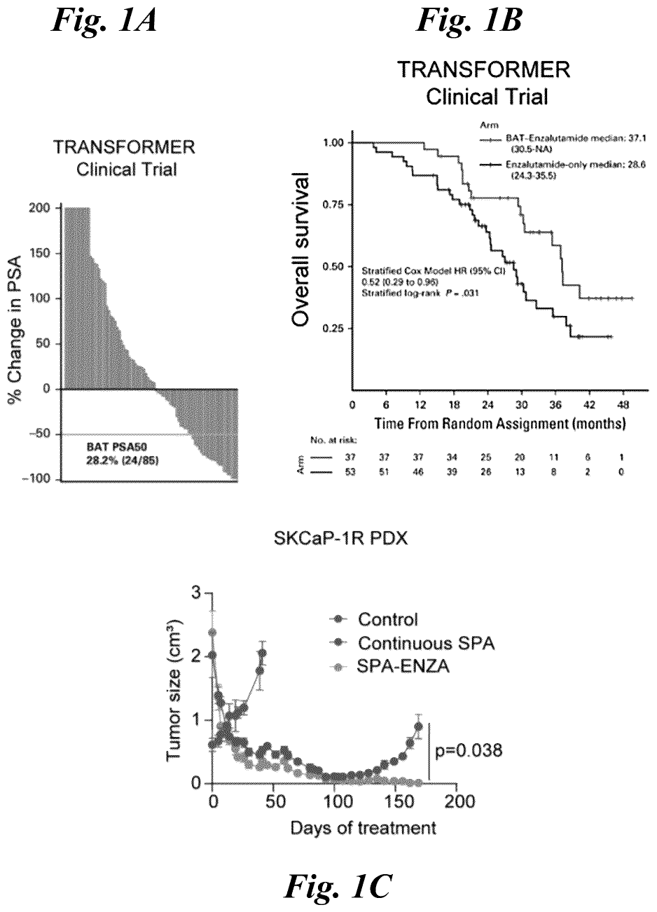

Inhibition of signaling through the androgen receptor (AR) is the mainstay of treatment of advanced PCa. Although these therapies are initially highly effective, castration-resistant PCa (CRPC) inevitably occurs. This phase of the disease is associated with poor prognosis and limited therapeutic options. The majority of PCas are addicted to signaling through AR and adaptively upregulate AR-activity, via mechanisms such as AR overexpression, to permit sustained AR signaling despite castration and AR inhibitors (1,2). Studies dating back nearly thirty years indicate that high expression of AR induced by prolonged castration becomes a vulnerability of CRPC cells in vitro and in mouse xenografts to supraphysiological androgen (SPA), which induces cell death and growth arrest in this context (3,4). On the basis of these studies, we developed an innovative treatment called Bipolar Androgen Therapy (BAT) for patients with CRPC, in which SPA is administered intermittently to result in cycling of serum testosterone from supraphysiological to near castrate levels. This rapid cycling is intended to reduce adaptation AR regulation associated with chronic exposure to high or low levels of testosterone, while simultaneously targeting the spectrum of AR expression present in heterogeneous CRPC tumors (5). We have now tested BAT in more than 350 patients with CRPC (6-10). These studies indicate that BAT is safe, improves quality of life, and produces biochemical and objective responses in 20-30% of patients (FIG. 1A).

Notably, several studies have suggested that patients who progress on BAT appear to be [re-]sensitized to subsequent AR inhibition (7-9). For example, in the TRANSFORMER trial, compared to patients who received enzalutamide without prior BAT, patients who received enzalutamide after prior BAT experienced greater PSA response (78% vs 25%), longer time to progression (10.9 vs 3.8 months) and significantly longer overall survival (37 vs 29 months, FIG. 1B). We recently described that high AR activity is required for tumor growth inhibition by BAT, and acquired resistance to BAT can be driven by downregulation of AR, which can be overcome by alternating AR stimulation with AR inhibition to prevent AR autoregulation (11). This process is illustrated in FIG. 1C, which demonstrates that more sustained growth inhibition of the CRPC patient-derived xenograft SKCaP-1R (12) can be achieved by alternating SPA with enzalutamide, rather than continuous treatment with SPA. Therefore, we have demonstrated the potential for greater control of CRPC growth by alternating BAT with enzalutamide.

### 1.3.2 SPA Stimulates Ornithine Decarboxylase (ODC) Activity Increasing Polyamine Concentrations

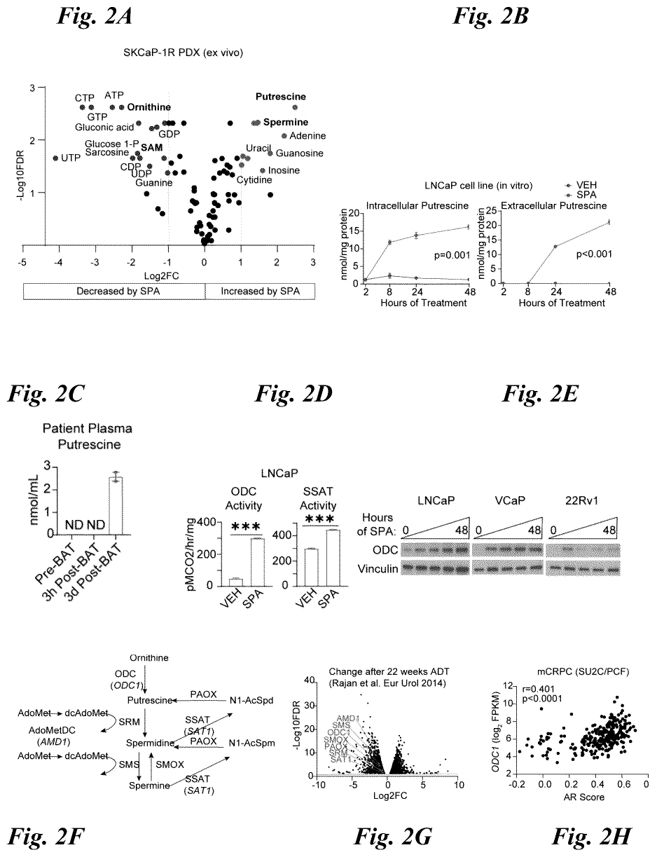

We have been studying how SPA affects the PCa cell and the cancer cell microenvironment with a goal of developing strategies to maximize growth-inhibitory effects and minimize adaptive effects. Global metabolomics of SKCaP-1R tumors after 14 days of SPA showed that the polyamines, putrescine and spermine, were the most enriched metabolites, while the polyamine precursors, ornithine and S-adenosylmethionine were among the most depleted (FIG. 2A). Marked intracellular and extracellular accumulation of putrescine was confirmed by HPLC in LNCaP cells treated with the synthetic androgen R1881 at the supraphysiological dose of 10 nM (FIG. 2B). Moreover, SPA increased plasma putrescine abundance in patients with CRPC (FIG. 2C). This increase in putrescine abundance likely occurs due to enhanced expression of the first rate-limiting enzyme in polyamine synthesis, ornithine decarboxylase (ODC), as SPA increased ODC protein and activity in PCa cell lines (FIG. 2D-FIG. 2F).

Beyond ODC, it is likely that multiple enzymes that promote polyamine synthesis and excretion are stimulated by AR activation, as spermidine/spermine N1-acetyltransferase (SSAT) activity also was significantly increased by SPA (FIG. 2D). Moreover, we observed that the genes coding for S-adenosylmethionine decarboxylase (AMD1), spermine synthase (SMS), ornithine decarboxylase (ODC1), spermine oxidase (SMOX), acetylpolyamine oxidase (PAOX), spermidine synthase (SRM), and spermidine/spermine N1-acetyltransferase (SAT1) were significantly downregulated in advanced PCa tumors after 22 weeks of ADT in the Rajan et al. dataset (13), and ODC1 expression positively correlated with AR activity in metastatic PCa tumors in the SU2C/PCF dataset (14). Together these data indicate that AR activation by SPA upregulates ODC to increase intracellular and excreted polyamines.

### 1.3.3 Inhibition of ODC Enhances Growth-Inhibitory Effects of SPA

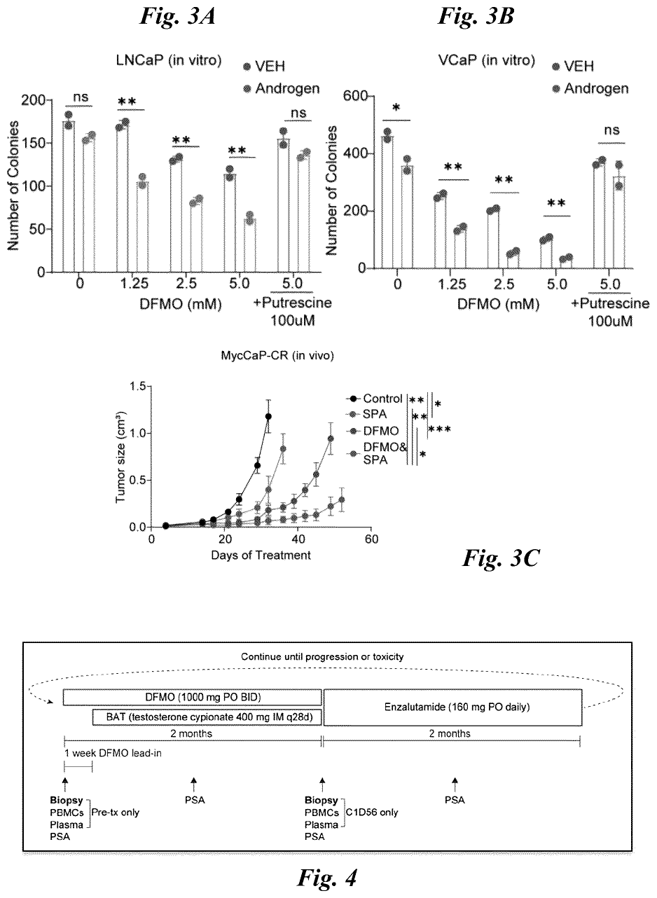

Polyamines are small, polycationic alkylamines that have a wide range of functions including stabilization of chromatin, maintenance of nucleic acid and protein synthesis, production of hypusine that modifies the translation elongation factor eIF5A, stimulation of MYC, regulation of pH, and modulation of nociception and immune responses (15). To assess the functional significance of stimulated polyamine synthesis by SPA, we tested the highly specific and clinically-utilized inhibitor of ODC, difluoromethylornithine (DFMO) (16). To assess additive antitumor effect through combination with DFMO, we treated LNCaP and VCaP PCa cells with a low dose of R1881 (0.5 nM, inhibits clonal survival by approximately 15%). DFMO inhibited clonal survival to a greater extent in R1881-treated cells than vehicle control-treated cells (FIG. 3A-FIG. 3b). Growth inhibition could be rescued by supplementing with exogenous putrescine (FIG. 3A-FIG. 3B), indicating that reduction of putrescine synthesis by DFMO increases growth suppression of CRPC by SPA. To assess whether combined therapy with SPA and DFMO has efficacy in vivo, we tested these agents in the subcutaneous castration-resistant syngeneic (immune-competent) mouse model, MYC-CaP-CR (17). This experiment indicated that combination treatment with SPA and DFMO was superior to either agent alone in reducing the rate of tumor growth in vivo (FIG. 3C). Altogether, these data suggest that SPA-stimulated synthesis of polyamines is protective for CRPC, and inhibiting this pathway using DFMO has potential to improve the efficacy of BAT.

### 1.3.4 History of Clinical Use of DEMO

The first phase I trial of DFMO was performed at our institution (Johns Hopkins) in 1984 for patients with advanced solid tumors or lymphomas (18), and shortly thereafter a phase I (5 patient) trial of DFMO and MGBG was performed for patients with advanced PCa in 1986 (19). It should be noted that DFMO was one of the first drugs to be approved for the treatment of Trypanosoma gambiense (African sleeping sickness) and was found to be extremely well-tolerated, even at high doses (20,21). Most recent clinical drug development of DFMO in cancer, however, has focused on assessing its potential as a chemo-preventive agent for cancer, owing to data that indicated elevated polyamines are permissive for cancer development (22). These trials suggest DFMO may reduce the incidence of non-melanoma skin cancers, but trials of breast and cervix cancer were negative (23-25). Large chemoprevention trials of DFMO for PCa have not been performed; however, initial studies suggest that DFMO can reduce prostate polyamine levels and the rate of growth of the prostate over one year (26,27).

This extensive clinical experience using DFMO provides a wealth of dosing and safety data on this drug, and indicate that it is effective at inhibiting ODC and is generally well tolerated. While high doses of DFMO are associated with reversible ototoxicity and myelosuppression, doses less than 1 g/m2/d are not consistently associated with these side-effects (28). Most recent clinical evaluation of DFMO as a cancer therapy has been limited to CNS tumors (29,30), and DEMO has never been assessed for efficacy as treatment for PCa. Our preclinical data presented here provide strong rationale to repurpose this drug as a combination therapy with BAT for the treatment of CRPC.

1.3.5 DFMO has Potential to Improve Pain and Quality of Life for Patients with Advanced PCa

Prior studies have suggested that elevated polyamines occurring in the inflammatory microenvironment mediate nociception in part through activating the capsaicin receptor TRPV1 (31-34). Testosterone administration is described to induce pain flares in patients with PCa (35), and we have occasionally seen this occur in our clinical studies. For this reason, all clinical trials of BAT have been limited to asymptomatic patients without pain requiring opiates. Without wishing to be bound to any one particular theory, it is thought that pain flares induced by BAT in a small subset of patient, may be due in part to enhanced polyamine production in the tumor microenvironment. Therefore, DFMO has the potential to prevent pain for patients on this trial by reducing polyamine abundance in metastatic tumors.

### 1.4. Approach

1.4.1 Conduct a Biomarker-Rich Phase II Trial of Sequential Repeat Cycles of BAT+DFMO Followed by Enzalutamide in Patients with mCRPC

### 1.4.1.1 Hypothesis

DFMO will augment the efficacy of BAT by prohibiting stimulation of polyamine synthesis by BAT. Moreover, DFMO and BAT will improve quality of life for patients by reducing pain due to bony metastases.

### 1.4.1.2 Overview

The proposed study is an open-label, single arm, phase 2 trial of repeat cycles of BAT in combination with DFMO alternating with enzalutamide for 50 patients with mCRPC progressing on abiraterone. A minimum of 20 patients with mCRPC amenable to biopsy will be enrolled. One cycle will involve a 7-day DFMO lead-in phase (to achieve ODC inhibition and polyamine depletion prior to BAT), followed by DFMO in combination with BAT for 2 months, followed by enzalutamide monotherapy for 2 months (FIG. 4). This 2-month cycling interval was selected because it is identical to the cycling interval of BAT and Enza in the ongoing STEPUP trial. This treatment protocol will allow us to compare results from similar correlative studies in men treated with BAT alone versus men treated with the combination of BAT and DFMO. Patients will be maintained on LHRH analogue throughout the study as per prior trials of BAT and standard-of-care for enzalutamide. Patients will be assessed monthly with a clinic visit, safety labs (CBC/CMP), PSA, and toxicity assessments. To monitor for ototoxicity due to DFMO, audiograms will be performed every 6 months while on DFMO. CT and bone scans will be performed every 2 months for the first 2 cycles then every 4 months thereafter. Treatment will be continued until PSA progression (PCGW3 criteria), clinical/radiographic progression, or toxicity requiring drug cessation. For correlative studies, metastatic tumor biopsies, peripheral blood mononuclear cells (PBMCs), and plasma will be collected at baseline and on C1D56 (i.e., after DFMO and BAT treatment).

### 1.4.1.3 Primary Endpoint

The primary endpoint of the study will be determined by the PSA response rate (>50% PSA decline from baseline) by C1D56.

### 1.4.1.4 Secondary Endpoints

Secondary endpoints include, but are not limited to: (1) PSA response rate (>50% PSA decline from baseline) at any point on trial; (2) incidence and severity of adverse events and serious adverse events graded according to CTCAE v4.0; (3) PSA progression-free survival (PSA-PFS) [PCWG3 criteria]; (4) progression-free survival (PFS): time to radiographic or clinical progression or death [PCWG3 definition]; (5) objective response rate in those with measurable disease; (6) durable PFS: Lack of clinical/radiographic progression for ≥6 months; (7) change in pain score on the short-form McGill Pain Questionnaire (SF-MPQ); and (8) overall survival.

### 1.4.1.5 Abbreviated Inclusion Criteria

Inclusion criteria include, but are not limited to: (1) metastatic CRPC, with serological and/or clinical/radiographic progression; (2) prior treatment with Abiraterone; and (3) an ECOG score of 0-1.

### 1.4.1.6 Abbreviated Exclusion Criteria

Abbreviated exclusion criteria include, but are not limited to: (1) no bone pain/symptomatic disease requiring opiate medications; (2) no liver metastases >2 cm in short-axis diameter; and (3) no prior enzalutamide or other second generation antiandrogens.

### 1.4.1.7 Metastatic Biopsies

We will use the biopsy standard operating procedures (SOPs) developed by the StandUp2Cancer initiative to collect image-guided metastatic soft-tissue biopsies. A minimum of 20 patients with soft tissue metastases amenable to biopsy will be enrolled, and biopsies will be obtained pre-treatment and on C1D56 after treatment with BAT and DFMO. Briefly, for each soft tissue metastatic biopsy, an 18-gauge core biopsy needle will be used to obtain ≥1 cores for FFPE (first priority), ≥1 fresh cores for immediate processing for single cell RNAseq (second priority), and ≥1 fresh cores for flash-freezing in liquid nitrogen (third priority). Previously we successfully obtained serial biopsies from 44 men on the COMBAT CRPC study and developed infrastructure to collect, process and laser-microdissect these specimens for multiplex IHC, RNAseq, WGS11.

### 1.4.1.8 Statistical Considerations

The PSAso response rate to 56 days of BAT monotherapy is approximately 25% in patients with mCRPC (6-9). We expect that the addition of DFMO to BAT will increase the PSA₅₀ response rate on C1D56 to 40%. A sample size of 46 patients therefore provides 81% power to reject a 25% response rate in favor of a 40% response rate, using a one-sided exact test with type-I error of 0.1. Accounting for an approximate 10% drop-out rate before evaluation, the trial will enroll 50 patients. Based on our prior experience with BAT studies in this patient population, we project to enroll 3-4 patients/month at Johns Hopkins and University of Minnesota allowing for enrollment completion over 12-16 months. The PSA response rate, defined as the proportion of evaluable patients who have ≥50% PSA decline from baseline, will be reported along with 95% confidence intervals. A patient will be considered evaluable if he receives at least one dose of DFMO in combination with BAT. Secondary efficacy endpoints (PSA-PFS, PFS and OS) will each be summarized using the Kaplan-Meier approach and log-rank test. Toxicity and adverse events will be tabulated by type and grade according to CTCAE v4.0.

### 1.4.1.9 Potential Challenges

DFMO may not enhance the response rate to BAT, but rather enhance the durability of response (i.e., PFS). Thus, PFS is a key secondary endpoint. Another challenge is that we are proposing a single-arm trial lacking control arms. This challenge is mitigated in part by our extensive experience with BAT and our ability to compare efficacy against patients treated similarly in the ongoing STEP-UP trial of alternating BAT and Enza, as well as historical controls from our trial assessing BAT and enzalutamide monotherapy. Our preliminary data indicate that the efficacy of DFMO is markedly improved in the presence of androgen. In patients, however, it is possible that DFMO has activity as monotherapy as well. Thus, should this proof-of-concept trial achieve the primary endpoint, it will not be clear whether the higher response rate of combination therapy is due to use of two therapies with independent anti-tumor activity or an interaction between these therapies as hypothesized. This result would strongly support subsequent larger controlled trials of these therapies.

### 1.4.2 Assess the Ability of DFMO to Inhibit Polyamine Production and Abundance Leading to Enhanced Downregulation of MYC and Epigenetic Reprogramming in the Presence of SPA

### 1.4.2.1 Goal/Hypothesis

This aspect of the study will confirm the mechanism of action of DFMO and assess the cancer cell-intrinsic effects of DFMO+BAT. Without wishing to be bound to any one particular theory, it is thought that DFMO will inhibit ODC leading to reduced abundance of putrescine in tumors and PBMCs of patients on trial. DFMO and BAT will lead to marked suppression of MYC and impairment of epigenetic regulation of gene expression leading to tumor regression.

### 1.4.2.2 Background

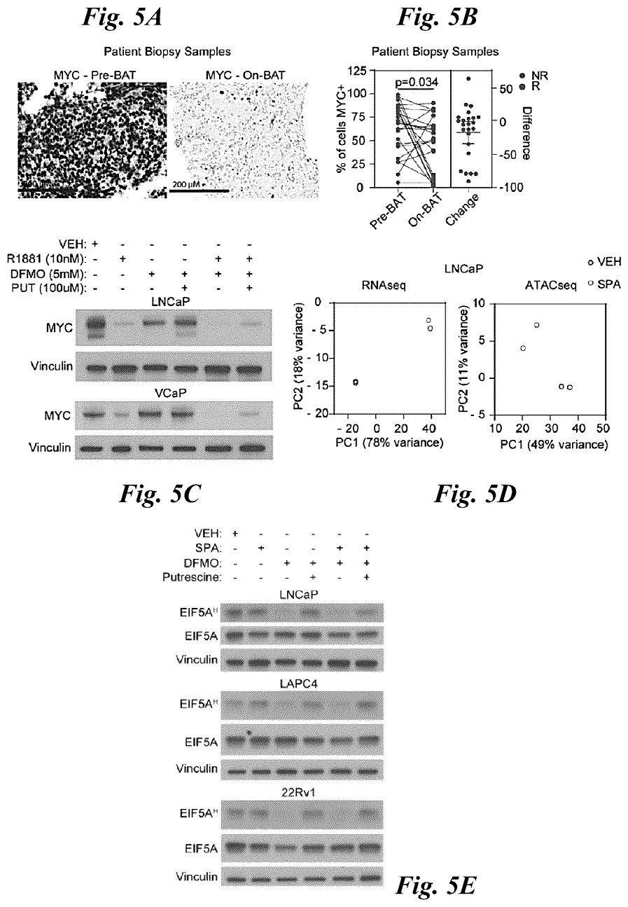

We recently completed the PCF-supported COMBAT study to dissect molecular mechanisms that drive the efficacy of BAT (11). The key finding of this study is that tumors with higher AR activity are sensitive to growth inhibition by BAT, which occurs in part through downregulation of MYC (FIG. 5A-FIG. 5B). Our work supports the widely recognized idea that MYC is a key driver of PCa progression (36-40) and effectively inhibiting MYC should be a top priority for PCa therapy development (41). While the ODC gene is a well-known transcriptional target of MYC (42), some reports suggest that polyamines also can stimulate MYC expression, participating in a feed-forward loop in some contexts (43-45).

To assess whether polyamines can drive MYC expression in PCa, we assessed MYC expression in LNCaP and VCaP cells treated with SPA and/or DFMO (FIG. 5C). SPA markedly reduced MYC expression. DFMO also reduced MYC, with near-complete ablation of MYC expression when used in combination with SPA. Notably, inhibition of MYC by DFMO could be rescued by putrescine supplementation, indicating that DFMO inhibits MYC by reducing polyamines. Without wishing to be bound to any one particular theory, it is thought that the surge in polyamines induced by BAT counteracts BAT efficacy in part by augmenting MYC, and the dual marked suppression of MYC by SPA and DEMO is in part responsible for the efficacy of this combination therapy.

The mechanism by which DFMO inhibits MYC is unknown, however ODC inhibition/deletion is described to alter global gene transcription due to dysregulation of chromatin accessibility and epigenetic programs, which may occur in part due to altered abundance of S-adenosylmethionine (SAM; used for polyamine synthesis by AdoMetDC), required for histone, DNA, and RNA methylation, and acetyl-coA (used for polyamine catabolismby SSAT) (FIG. 2F) (46-51).

We have observed that PCa response to SPA results in marked changes in the transcriptome and chromatin accessibility (FIG. 5D) and hypothesize that DFMO will obstruct global transcriptional nimbleness required for therapy adaptation and resistance. Lastly, spermidine is the substrate for production of the amino acid hypusine, which is covalently bound to and required for activity of the elongation factor cIF5A. Hypusinated-eIF5A promotes tumor growth by promoting accumulation of proteins such as RhoA and PEAK1 (52-54). DFMO monotherapy or in combination with SPA resulted in marked reduction of eIF5A hypusination in PCa cell lines (FIG. 5E), which we predict will further suppress PCa growth.

### 1.4.2.3 Research Design

Metastatic tumor biopsy samples will be collected prior to treatment and on C1D56 (i.e., following DFMO+BAT) for a minimum of 20 patients on trial (n=40 total biopsies) and divided for (1) FFPE for immunohistochemistry (2) immediate processing for single cell RNAseq, and (3) flash freezing for global metabolomics. Libraries for scRNAseq will be immediately prepared for all samples received, however, sequencing and metabolomics will only proceed after pathologic examination of adjacent core shows ≥10% cancer for scRNAseq and ≥50% cancer for metabolomics. We anticipate that 25-40% of patients will experience a PSA response to treatment, equating to 4-8 paired biopsies from responding patients. PBMCs and plasma will be collected prior to treatment and on C1D56 for all patients on trial.

To confirm that DFMO reduces activity of ODC to result in decreased abundance of putrescine, we will measure ODC activity and polyamine concentrations in metastatic biopsies when possible, and in PBMCs, as we have previously published (55-57). PBMCs are used here as a surrogate for tumor tissue given that PBMCs will be available in abundance for all patients at both time points. Native and acetylated polyamine plasma concentrations on C1D56, which are likely derived from tumor, will be compared with baseline samples and with historical controls from patients treated with BAT monotherapy at a similar time point (FIG. 2C). In patients with paired biopsies, IHC for ODC and global metabolomics will be performed and will be used to confirm increased ODC expression and decreased abundance of polyamines on C1D56.

Next, we will study cancer cell-intrinsic molecular consequences of BAT+DFMO. First, we will perform hypothesis-directed analyses, then we will perform unbiased analyses of the large metabolomic and transcriptomic datasets collected. To assess whether BAT+DFMO will result in enhanced suppression of MYC compared with BAT monotherapy, MYC protein expression will be assessed by IHC of biopsy samples with quantitative image analysis (as per FIG. 5A), and the percentage of patients with >50% decrease in MYC will be compared with our prior trial of BAT monotherapy (11). To confirm this result, we will assess the change in RNA expression of MYC target gene sets within cancer cells in our scRNAseq dataset from tumor biopsies. To infer how BAT+DFMO might affect epigenetic programs, we will assess whether therapy affects abundance of acetyl-coA and SAM measured by global metabolomics of tumor and epigenetic marks on histone H3 protein by IHC of tumor, including activating marks such as H3k4me3, H3k9Ac, H3k27Ac, and H3k36me3, and the H3k9me3 repressive mark, as we have previously done (58,59). To assess the effect of therapy on eIF5A hypusination, we will perform IHC of tumor for hypusinated eIF5A (60). Unbiased analyses of metabolomic and transcriptomic datasets will be done:

### 1.4.2.4 Untargeted Metabolomics

Flash frozen samples will be provided to a laboratory who will perform sample preparation, extraction of metabolites, high performance liquid chromatography/tandem mass spectrometry in positive and negative ion modes using a combination reverse phase and HILIC chromatography method with metabolite identification using software with a 5800-compound reference library. Data processing will be performed using the software Metaboscape (Bruker). Statistical analyses and graphical visualization will be performed using MetaboAnalyst 5.0. Differential expression analysis will be performed using single sample Bayes moderated t-statistics as described (61) to identify changes in metabolite levels on C1D56. Statistics for each metabolite will be reported along with Benjamini-Hochberg adjusted p-values to account for multiple tests. FDRs below 0.1 will be considered significant. Changes in metabolite expression after treatment, as well as absolute metabolite expression (1) at baseline and (2) after treatment, will be correlated to clinical outcomes of PFS and OS using Cox-regression, and to change in PSA, using linear regression.

### 1.4.2.5 Single Cell RNAseq

Biopsy samples will be prepared as libraries and sequenced using a 10X Chromium controller with the Chromium Next GEM Single Cell 3′ Reagent Kits v3.1 and a NovaSeq (Illumina). Sequenced libraries will be aligned using Cell Ranger 2.2 (10X genomics) to obtain a raw read count matrix of barcodes corresponding to cells and features corresponding to detected genes. Read count matrices will be processed, analyzed and visualized with machine learning algorithms in R v. 4.0.0 (R Core Team 2013) using Scurat v. 4 (62). Filtered samples will be normalized using a regularized negative binomial regression (63) and integrated with the reciprocal principal component analysis (rpca) approach followed by mutual nearest neighbors. Integrated gene expression matrices will be visualized with a Uniform Manifold Approximation and Projection (UMAP) (64) as a dimensionality reduction approach. Cell clustering will be performed with the Leiden approach (65). Resolution for cell clustering will be determined by evaluating hierarchical clustering trees at a range of resolutions (0-1.2) with Clustree (66), selecting a value inducing minimal cluster instability. Cell clusters will be mapped to cell identities using a machine learning automated approach, SingleR (67), then curated manually, and cell-type/cluster specific genes will be explored for pathway enrichment using StringDB (68) and goseq (71). Changes in cluster abundance after treatment, as well as absolute cluster abundance (1) at baseline and (2) after treatment, will be correlated to clinical outcomes of PFS and OS using Cox-regression, and to change in PSA, using linear regression.

### 1.4.2.6 Potential Challenges

This aspect of the study is designed to verify on-target effects of DFMO, followed by hypothesis-directed and unbiased analyses of cancer cell-intrinsic effects of DFMO&BAT. Given prior studies that indicate robust inhibition of ODC and decrease in putrescine by DFMO used at similar doses (26,27), we anticipate that DFMO will exhibit potent inhibition of ODC. A potential pitfall is the number of tumor biopsies planned (20 paired biopsies). This number was selected as a balance between maximizing power for assessment of treatment effect and the depth of analyses per sample due to budgetary constraints. Another challenge is that DFMO may inhibit ODC but not lead to decreased abundance of polyamines due to compensatory increased uptake. This would be inferred if ODC is inhibited but polyamine abundance is not reduced. Cycling DFMO on and off every two months may minimize this potential compensatory uptake. Another pitfall is that it may be challenging to quantify histone marks by IHC. We have done this previously for H3k36me3 mark (59), but not with other marks. Should optimization of antibody staining prove challenging we will proceed only with assessment of top three of interest determined in clinical studies.

### 1.4.3 Evaluate the Ability of DFMO to Augment a Tumor Immune Response in the Presence of SPA

### 1.4.3.1 Hypothesis

Without wishing to be bound to any one particular theory, it is thought that DFMO and BAT will enhance the abundance and inflammatory phenotype of intratumoral immune cells, and inflammatory phenotype and function of circulating immune cells in patients.

### 1.4.3.2 Background

Polyamines exist in high concentrations in sites of inflammation (69,70) where they appear to play a role in resolution of inflammation (71). Specifically, spermine and spermidine dampen production of proinflammatory cytokines in macrophages and drive differentiation and survival of alternatively-activated immunosuppressive M2 macrophages and myeloid-derived suppressor cells (51,72-77). Recent work indicates that polyamines also are required for T cell lineage commitment and restraint of T cell inflammatory cytokine production, with deletion of ODC in T cells driving robust production of IFNγ (irrespective of culturing conditions to guide lineage differentiation) and autoimmune colitis in mice (47). The microenvironment of metastatic prostate cancer is highly immunosuppressive (i.e., immunologically cold) (78). Without wishing to be bound to any one particular theory, it is thought that this immunosuppressive microenvironment is in part driven by production and secretion of polyamines by CRPC (79), which may be exacerbated when patients are treated with BAT.

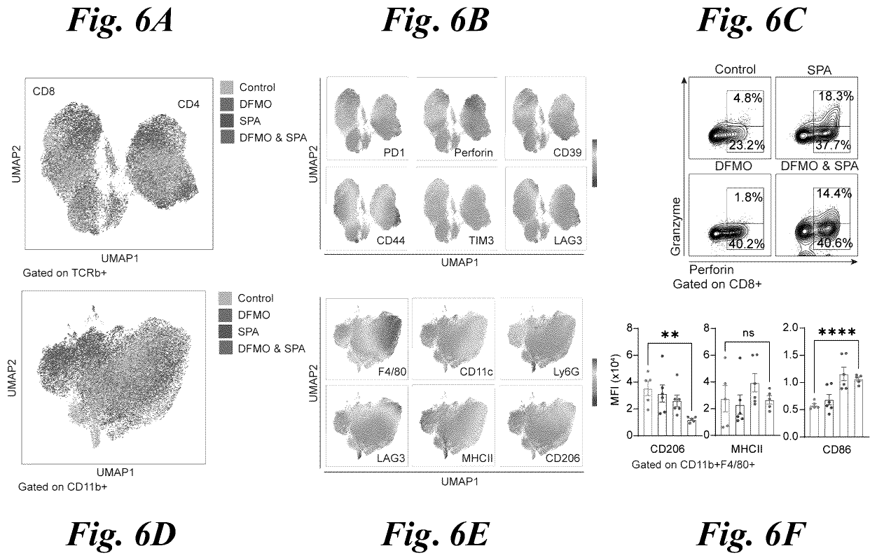

We observed that combination treatment of SPA and DFMO of castrated immune-competent FVB mice bearing MYC-CaP-CR resulted in markedly greater suppression of tumor growth than control or either drug given as monotherapy (FIG. 3C). To characterize how SPA+DFMO alters phenotypes of tumor-infiltrating immune cells, we performed high dimensional flow cytometry of these tumors after approximately 3 weeks of treatment using a panel of 30 markers of immune cell lineage and functional phenotype. We noted marked differences in the phenotype of tumor-infiltrating T cells due to treatment (FIG. 6A) driven primarily by expression of activation markers including perforin, CD39, CD44, and LAG3 (FIG. 6B). SPA+DFMO treatment enhanced the percentage of CD8 T cells expressing granzyme and perforin (FIG. 6D), indicating enhanced cytotoxic function by combination treatment. Notable differences in myeloid cell phenotype were also observed (FIG. 6D), driven primarily by altered macrophage markers (FIG. 6E), with reduction of the M2 macrophage marker CD206 and enhancement of the M1 macrophage marker CD86 (FIG. 6F). Together this experiment suggests that SPA+DFMO enhances inflammatory phenotypes of tumor-infiltrating phenotypes and supports our hypothesis that DFMO will augment an antitumor immune response in patients on trial.

### 1.4.3.3 Research Design

We will assess how BAT+DFMO alters immune cells in the tumor and in circulation. Analysis of intratumoral immune cells will reveal how therapy alters the tumor immune microenvironment, while analysis of circulating immune cells will allow us to measure changes to immune cell function (e.g., cytokine production, cytotoxicity, T cell suppression).

First, we will utilize the scRNAseq of tumor biopsies from section 1.4.1.7 to determine abundance and gene expression of intratumoral immune cell populations. Integrated gene expression matrices with UMAP approach will be performed for CD45+ cells, with cell clustering by the Leiden approach as above (65). Resolution for cell clustering will be determined by evaluating hierarchical clustering trees at a range of resolutions (0-1.2) with Clustree (66), selecting a value inducing minimal cluster instability. Cell clusters will be mapped to cell identities using a machine learning automated approach, SingleR (67), then curated manually, and cell-type/cluster specific genes will be explored for pathway enrichment using StringDB (68) and goseq (71). Changes in cluster abundance after treatment, as well as absolute cluster abundance (1) at baseline and (2) after treatment, will be correlated to clinical outcomes of PFS and OS using Cox-regression, and to change in PSA, using linear regression. Significant changes in immune cell populations will be validated by IHC of biopsy samples.

Next, we will characterize changes to circulating immune cell populations and assess congruency with tumor immune cells. PBMCs will be collected and cryopreserved pre-treatment and on C1D56 for all patients on trial. PBMCs will be evaluated by high dimensional spectral flow cytometry using the Cytec Aurora, which can decipher up to 40 different markers simultaneously. We will utilize optimized panels to assess circulating immune cell subsets, which can be tailored to detect specific markers found to be significantly altered by scRNAseq of tumor immune cells. Next, we will assess ex vivo immune cell function. CD8 T cells from n=4 responders and n=4 non-responders pre-treatment and on C1D54 will be isolated by FACS sorting and/or magnetic bead separation, stimulated with anti-CD3/anti-CD28 with/without skewing conditions, and cytokine expression assessed by flow cytometry. Similarly, MDSC populations from n=4 responders and n=4 non-responders pre-treatment and on C1D54 will be isolated by FACS sorting and/or magnetic bead separation and co-cultured with increasing numbers of CFSE-pre-incubated CD8 cells and CFSE dilution assessed following stimulation.

### 1.4.3.4 Potential Challenges

It is possible that polyamine production is a significant immune cell checkpoint in patients with mCRPC treated with BAT, however another immune cell checkpoint becomes activated following treatment with DFMO (e.g. PD-1/PD-L1), such that abundance and anti-tumor immune cell function is not significantly increased by treatment. A strength of our approach is that we will perform extensive characterization of immune cell changes such that we may identify this secondary barrier to tumor immunity to subsequently develop new strategies to overcome it. Another challenge is that our scRNAseq will be performed on bulk tumor samples, not CD45+-sorted samples. Given that mCRPC is known to have low quantities of immune cells (80), we may be limited in the analysis of low abundance clusters of immune cells. We reason these clusters are unlikely to have clinical significance given very low abundance. Another challenge is that immune cell function (particularly MDSC suppressive function) can be disturbed by freeze-thawing. If we determine that control MDSCs are poorly suppressive of T cell proliferation, we will perform MDSC suppression assays using freshly isolated PBMCs. Lastly, a potential challenge is that some tumors will be derived from lymph node with surrounding normal immune cells. To minimize this challenge we will assess adjacent cores histologically prior to sequencing and exclude samples with ≥25% normal lymph node tissue from immune cell analyses.

### 1.5 References Example 1

1. Linja, M. J. et al. Amplification and overexpression of androgen receptor gene in hormone-refractory prostate cancer. Cancer Res. 61, 3550-3555 (2001).
2. Scher, H. I. & Sawyers, C. L. Biology of progressive, castration-resistant prostate cancer: directed therapies targeting the androgen-receptor signaling axis. J. Clin. Oncol. 23, 8253-8261 (2005).
3. Kokontis, J., Takakura, K., Hay, N. & Liao, S. Increased androgen receptor activity and altered c-myc expression in prostate cancer cells after long-term androgen deprivation. Cancer Res. 54, 1566-1573 (1994).
4. Umekita, Y., Hiipakka, R. A., Kokontis, J. M. & Liao, S. Human prostate tumor growth in athymic mice: inhibition by androgens and stimulation by finasteride. Proc. Natl. Acad. Sci. U.S.A 93, 11802-11807 (1996).
5. Denmeade, S. R. & Isaacs, J. T. Bipolar androgen therapy: the rationale for rapid cycling of supraphysiologic androgen/ablation in men with castration resistant prostate cancer. Prostate 70, 1600-1607 (2010).
6. Teply, B. A. et al. Bipolar androgen therapy in men with metastatic castration-resistant prostate cancer after progression on enzalutamide: an open-label, phase 2, multicohort study. Lancet Oncol. 19, 76-86 (2018).
7. Markowski, M. C. et al. A multicohort open-label phase II trial of bipolar androgen therapy in men with metastatic castration-resistant prostate cancer (RESTORE): A comparison of post-abiraterone versus post-enzalutamide cohorts. Eur. Urol. (2020) doi:10.1016/j.eururo.2020.06.042.
8. Sena, L. A. et al. Bipolar androgen therapy sensitizes castration-resistant prostate cancer to subsequent androgen receptor ablative therapy. Eur. J. Cancer 144, 302-309 (2020).
9. Denmeade, S. R. et al. TRANSFORMER: A randomized phase II study comparing bipolar androgen therapy versus enzalutamide in asymptomatic men with castration-resistant metastatic prostate cancer. J. Clin. Oncol. JCO2002759 (2021).
10. Markowski, M. C. et al. COMBAT-CRPC: Concurrent administration of bipolar androgen therapy (BAT) and nivolumab in men with metastatic castration-resistant prostate cancer (mCRPC). J. Clin. Oncol. 39, 5014-5014 (2021).
11. Sena, L. A. et al. Prostate cancer androgen receptor activity dictates efficacy of Bipolar Androgen Therapy. bioRxiv (2022) doi:10.1101/2022.04.26.22274275.
12. Zhu, Y. et al. Role of androgen receptor splice variant-7 (AR-V7) in prostate cancer resistance to 2ndgeneration androgen receptor signaling inhibitors. Oncogene 39, 6935-6949 (2020).
13. Rajan, P. et al. Next-generation sequencing of advanced prostate cancer treated with androgen-deprivation therapy. Eur. Urol. 66, 32-39 (2014).
14. Abida, W. et al. Genomic correlates of clinical outcome in advanced prostate cancer. Proc. Natl. Acad. Sci. U.S.A 116, 11428-11436 (2019).
15. Casero, R. A., Jr, Murray Stewart, T. & Pegg, A. E. Polyamine metabolism and cancer: treatments, challenges and opportunities. Nat. Rev. Cancer 18, 681-695 (2018).
16. Prakash, N. J., Schechter, P. J., Grove, J. & Koch-Weser, J. Effect of alpha-difluoromethylornithine, an enzyme-activated irreversible inhibitor of ornithine decarboxylase, on L1210 leukemia in mice. Cancer Res. 38, 3059-3062 (1978).
17. Ellis, L., Lehet, K., Ramakrishnan, S., Adelaiye, R. & Pili, R. Development of a castrate resistant transplant tumor model of prostate cancer. Prostate 72, 587-591 (2012).
18. Abeloff, M. D. et al. Phase I trial and pharmacokinetic studies of alpha-difluoromethylornithine—an inhibitor of polyamine biosynthesis. J. Clin. Oncol. 2, 124-130 (1984).
19. Herr, H. W., Warrel, R. P. & Burchenal, J. H. Phase I trial of α-difluoromethyl ornithine (DFMO) and methylglyoxal bis(guanylhydrazone) (MGBG) in patients with advanced prostatic cancer. Urology 28, 508-511 (1986).
20. McCann, P. P., Bitonti, A. J., Bacchi, C. J. & Clarkson, A. B., Jr. Use of difluoromethylornithine (DFMO, eflornithine) for late-stage African trypanosomiasis. Trans. R. Soc. Trop. Med. Hyg. 81, 701-702 (1987).
21. Doua, F. et al. Treatment of human late stage gambiense trypanosomiasis with alpha-difluoromethylornithine (eflornithine): efficacy and tolerance in 14 cases in Côte d'Ivoire. Am. J. Trop. Med. Hyg. 37, 525-533 (1987).
22. Shukla-Dave, A. et al. Ornithine decarboxylase is sufficient for prostate tumorigenesis via androgen receptor signaling. Am. J. Pathol. 186, 3131-3145 (2016).
23. Alberts, D. S. et al. Chemoprevention of human actinic keratoses by topical 2-(difluoromethyl)-dl-ornithine. Cancer Epidemiol. Biomarkers Prev. 9, 1281-1286 (2000).
24. Fabian, C. J. et al. A phase II breast cancer chemoprevention trial of oral alpha-difluoromethylornithine: breast tissue, imaging, and serum and urine biomarkers. Clin. Cancer Res. 8, 3105-3117 (2002).
25. Vlastos, A.-T. et al. Results of a phase II double-blinded randomized clinical trial of difluoromethylornithine for cervical intraepithelial neoplasia grades 2 to 3. Clin. Cancer Res. 11, 390-396 (2005).
26. Simoneau, A. R., Gerner, E. W., Phung, M., McLaren, C. E. & Meyskens, F. L.—difluoromethylornithine and polyamine levels in the human prostate: Results of a phase IIa trial. J. Natl. Cancer Inst. 93, 57-59 (2001).
27. Simoneau, A. R. et al. The effect of difluoromethylornithine on decreasing prostate size and polyamines in men: results of a year-long phase IIb randomized placebo-controlled chemoprevention trial. Cancer Epidemiol. Biomarkers Prev. 17, 292-299 (2008).
28. Doyle, K. J., McLaren, C. E., Shanks, J. E., Galus, C. M. & Meyskens, F. L. Effects of difluoromethylornithine chemoprevention on audiometry thresholds and otoacoustic emissions. Arch. Otolaryngol. Head. Neck Surg. 127, 553-558 (2001).
29. Levin, V. A. et al. Phase III randomized study of postradiotherapy chemotherapy with alpha-difluoromethylornithine-procarbazine, N-(2-chloroethyl)-N′-cyclohexyl-N-nitrosurea, vincristine (DFMOPCV) versus PCV for glioblastoma multiforme. Clin. Cancer Res. 6, 3878-3884 (2000).
30. Levin, V. A. et al. Phase III randomized study of postradiotherapy chemotherapy with combination alpha-difluoromethylornithine-PCV versus PCV for anaplastic gliomas. Clin. Cancer Res. 9, 981-990 (2003).
31. Tan-No, K. et al. Intrathecally administered spermine produces the scratching, biting and licking behaviour in mice. Pain 86, 55-61 (2000).
32. Ahern, G. P., Wang, X. & Miyares, R. L. Polyamines are potent ligands for the capsaicin receptor TRPV1. J. Biol. Chem. 281, 8991-8995 (2006).
33. Rivat, C. et al. Polyamine deficient diet to relieve pain hypersensitivity. Pain 137, 125-137 (2008).
34. Silva, M. A. et al. Role of peripheral polyamines in the development of inflammatory pain. Biochem. Pharmacol. 82, 269-277 (2011).
35. Tagnon, H. J., Schulman, P., Whitmore, W. F. & Leone, L. A. Prostatic fibrinolysin. Am. J. Med. 15, 875-884 (1953).
36. Gurel, B. et al. Nuclear MYC protein overexpression is an early alteration in human prostate carcinogenesis. Mod. Pathol. 21, 1156-1167 (2008).
37. Koh, C. M. et al. MYC and prostate cancer. Genes Cancer 1, 617-628 (2010).
38. Koh, C. M. et al. Alterations in nucleolar structure and gene expression programs in prostatic neoplasia are driven by the MYC oncogene. Am. J. Pathol. 178, 1824-1834 (2011).
39. Han, H. et al. Small-molecule MYC inhibitors suppress tumor growth and enhance immunotherapy. Cancer Cell 36, 483-497.e15 (2019).
40. Qiu, X. et al. MYC drives aggressive prostate cancer by disrupting transcriptional pause release at androgen receptor targets. Nat. Commun. 13, 2559 (2022).
41. Truica, M. I., Burns, M. C., Han, H. & Abdulkadir, S. A. Turning up the heat on MYC: Progress in small-molecule inhibitors. Cancer Res. 81, 248-253 (2021).
42. Bello-Fernandez, C., Packham, G. & Cleveland, J. L. The ornithine decarboxylase gene is a transcriptional target of c-Myc. Proc. Natl. Acad. Sci. U.S.A 90, 7804-7808 (1993).
43. Celano, P., Baylin, S. B. & Casero, R. A., Jr. Polyamines differentially modulate the transcription of growth-associated genes in human colon carcinoma cells. J. Biol. Chem. 264, 8922-8927 (1989).
44. Patel, A. R. & Wang, J. Y. Polyamines modulate transcription but not posttranscription of c-myc and c-jun in IEC-6 cells. Am. J. Physiol. 273, C1020-9 (1997).
45. Liu, L. et al. Polyamine-modulated c-Myc expression in normal intestinal epithelial cells regulates p21Cip1 transcription through a proximal promoter region. Biochem. J. 398, 257-267 (2006).
46. Arruabarrena-Aristorena, A., Zabala-Letona, A. & Carracedo, A. Oil for the cancer engine: The cross-talk between oncogenic signaling and polyamine metabolism. Sci. Adv. 4, eaar2606 (2018).
47. Puleston, D. J. et al. Polyamine metabolism is a central determinant of helper T cell lineage fidelity. Cell 184, 4186-4202.e20 (2021).
48. Li, H. et al. YAP/TAZ drives cell proliferation and tumour growth via a polyamine-eIF5A hypusination-LSD1 axis. Nat. Cell Biol. 24, 373-383 (2022).
49. Hobbs, C. A. & Gilmour, S. K. High levels of intracellular polyamines promote histone acetyltransferase activity resulting in chromatin hyperacetylation. J. Cell. Biochem. 77, 345 (2000).
50. Morselli, E. et al. Spermidine and resveratrol induce autophagy by distinct pathways converging on the acetylprotcomc. J. Cell Biol. 192, 615-629 (2011).
51. Hardbower, D. M. et al. Ornithine decarboxylase regulates M1 macrophage activation and mucosal inflammation via histone modifications. Proc. Natl. Acad. Sci. U.S.A. 114, E751-E760 (2017).
52. Fujimura, K. et al. A hypusine-eIF5A-PEAK1 switch regulates the pathogenesis of pancreatic cancer. Cancer Res. 74, 6671-6681 (2014).
53. Mémin, E. et al. Blocking eIF5A modification in cervical cancer cells alters the expression of cancer-related genes and suppresses cell proliferation. Cancer Res. 74, 552-562 (2014).
54. Muramatsu, T. et al. The hypusine cascade promotes cancer progression and metastasis through the regulation of RhoA in squamous cell carcinoma. Oncogene 35, 5304-5316 (2016).
55. Casero, R. A., Jr et al. Differential induction of spermidine/spermine N1-acetyltransferase in human lung cancer cells by the bis(ethyl) polyamine analogues. Cancer Res. 49, 3829-3833 (1989).
56. Giardiello, F. M. et al. Ornithine decarboxylase and polyamines in familial adenomatous polyposis. Cancer Res. 57, 199-201 (1997).
57. Cruz-Correa, M. et al. Efficacy and safety of curcumin in treatment of intestinal adenomas in patients with familial adenomatous polyposis. Gastroenterology 155, 668-673 (2018).
58. Seligson, D. B. et al. Global histone modification patterns predict risk of prostate cancer recurrence. Nature 435, 1262-1266 (2005).
59. Pellakuru, L. G. et al. Global levels of H3K27me3 track with differentiation in vivo and are deregulated by MYC in prostate cancer. Am. J. Pathol. 181, 560-569 (2012).
60. Cracchiolo, B. M. et al. Eukaryotic initiation factor 5A-1 (eIF5A-1) as a diagnostic marker for aberrant proliferation in intraepithelial neoplasia of the vulva. Gynecol. Oncol. 94, 217-222 (2004).
61. Kammers, K., Cole, R. N., Tiengwe, C. & Ruczinski, I. Detecting significant changes in protein abundance. EuPA Open Proteom. 7, 11-19 (2015).
62. Stuart, T. et al. Comprehensive integration of single-cell data. Cell 177, 1888-1902.e21 (2019).
63. Hafemeister, C. & Satija, R. Normalization and variance stabilization of single-cell RNA-seq data using regularized negative binomial regression. Genome Biol. 20, 296 (2019).
64. McInnes, L., Healy, J. & Melville, J. UMAP: Uniform Manifold Approximation and Projection for Dimension Reduction. arXiv [stat.ML] (2018).
65. Traag, V. A., Waltman, L. & van Eck, N. J. From Louvain to Leiden: guaranteeing well-connected communities. Sci. Rep. 9, 5233 (2019).
66. Zappia, L. & Oshlack, A. Clustering trees: a visualization for evaluating clusterings at multiple resolutions. Gigascience 7, (2018).
67. Aran, D. et al. Reference-based analysis of lung single-cell sequencing reveals a transitional profibrotic macrophage. Nat. Immunol. 20, 163-172 (2019).
68. Szklarczyk, D. et al. STRING v11: protein-protein association networks with increased coverage, supporting functional discovery in genome-wide experimental datasets. Nucleic Acids Res. 47, D607-D613 (2019).
69. Yukioka, K. et al. Polyamine levels in synovial tissues and synovial fluids of patients with rheumatoid arthritis. J. Rheumatol. 19, 689-692 (1992).
70. Pillai, R. B. et al. Increased colonic ornithine decarboxylase activity in inflammatory bowel disease in children. Dig. Dis. Sci. 44, 1565-1570 (1999).
71. Puleston, D. J., Villa, M. & Pearce, E. L. Ancillary activity: Beyond core metabolism in immune cells. Cell Metab. 26, 131-141 (2017).
72. Van den Bossche, J. et al. Pivotal Advance: Arginase-1-independent polyamine production stimulates the expression of IL-4-induced alternatively activated macrophage markers while inhibiting LPS-induced expression of inflammatory genes. J. Leukoc. Biol. 91, 685-699 (2012).
73. Yang, Q. et al. Spermidine alleviates experimental autoimmune encephalomyelitis through inducing inhibitory macrophages. Cell Death Differ. 23, 1850-1861 (2016).
74. Chaturvedi, R. et al. Polyamines impair immunity to Helicobacter pylori by inhibiting L-arginine uptake required for nitric oxide production. Gastroenterology 139, 1686-98, 1698.e1-6 (2010).
75. Haskó, G. et al. Spermine differentially regulates the production of interleukin-12 p40 and interleukin-10 and suppresses the release of the T helper 1 cytokine interferon-gamma. Shock 14, 144-149 (2000).
76. Miska, J. et al. Polyamines drive myeloid cell survival by buffering intracellular pH to promote immunosuppression in glioblastoma. Sci. Adv. 7, eabc8929 (2021).
77. Travers, M. et al. DFMO and 5-azacytidine increase M1 macrophages in the tumor microenvironment of Murine ovarian cancer. Cancer Res. 79, 3445-3454 (2019).
78. Sena, L. A., Denmeade, S. R. & Antonarakis, E. S. Targeting the spectrum of immune checkpoints in prostate cancer. Expert Rev. Clin. Pharmacol. 1-14 (2021).
79. Holbert, C. E., Cullen, M. T., Casero, R. A., Jr & Stewart, T. M. Polyamines in cancer: integrating organismal metabolism and antitumour immunity. Nat. Rev. Cancer (2022) doi:10.1038/s41568-022-00473-2.
80. He, M. X. et al. Transcriptional mediators of treatment resistance in lethal prostate cancer. Nat. Med. 27, 426-433 (2021).

### Example 2

### The Role of Mitochondrial Metabolism in Prostate Cancer Progression and Susceptibility to Bipolar Androgen Therapy

### 2.1 Overview

Bipolar Androgen Therapy (BAT) is a novel treatment for patients with castration-resistant prostate cancer (CRPC) that is safe, improves quality of life, produces objective and sustained responses in a subset of patients, and may re-sensitize CRPC to subsequent AR ablative therapies. BAT consists of monthly injections of high dose testosterone (T) concurrent with ongoing treatment with an LHRH agonist to result in oscillation in serum T from supraphysiological to near-castrate levels. At this time, however, sustained antitumor benefit from BAT is restricted to a minority of patients with CRPC. A detailed understanding of effects of BAT on CRPC growth and function is needed to identify synergistic combination therapies that might expand the population of patients who benefit from BAT. The goal of this project is to define the effect of BAT on CRPC cell metabolism and determine whether inhibition of induced metabolic pathways in combination with BAT increases CRPC cell death and growth inhibition.

Testosterone is fundamentally a regulator of cellular metabolism—it is known for decades to induce anabolic metabolism in skeletal muscle, cardiac muscle, adipose tissue, and other tissues in the body. In normal prostate epithelial cells, T drives unique metabolic flux to promote secretion of citrate and polyamines into prostatic secretions, which support sperm survival and function in the female reproductive tract. When prostate epithelial cells are converted to prostate cancer, T continues to dictate metabolism, now supporting forward flux through the tricarboxylic acid (TCA) cycle to meet the biosynthetic and energetic demands of tumorigenesis. Therefore, without wishing to be bound to any one particular theory it is thought that high dose androgen (HDA) provided in BAT creates dependency on specific metabolic pathways including TCA cycle anaplerosis and polyamine synthesis to support induced anabolic metabolism and maintain cell viability. To test this hypothesis, we are utilizing CRPC cell lines, patient-derived xenograft (PDX) mouse models, and patient biopsy samples obtained before and after three months of BAT from the COMBAT trial (NCT03554317).

### 2.2 Introduction

This Example assesses molecular mechanisms of Bipolar Androgen Therapy (BAT) for castration-resistant prostate cancer with the goal of identifying synergistic therapeutic drug targets. The proposed work addresses, in part, the challenge of developing treatments that improve outcomes for men with lethal prostate cancer. We are using CRPC cell lines, patient-derived xenograft models, and paired patient biopsies before and after/during BAT from the COMBAT trial (NCT03554317) to define mechanisms of and vulnerabilities induce by BAT. This work has the potential to identify novel strategies using drug combinations to treat men with advanced prostate cancer to translate into clinical trials.

### 2.3 Results

### 2.3.1 Determination of the Metabolic Changes in Prostate Cancer Cells in Patients Receiving BAT

### 2.3.1.1 Collect Biopsy Samples

Samples were collected on this clinical trial. Currently 14 patients are enrolled. For this analysis, we will collect biopsy samples from 20 patients before and after testosterone treatment for a total of 40 samples. There will be no identifiable information on patient samples. We exceeded the goal to collect 20 paired biopsies, obtained before and after 3 months of testosterone treatment, and obtained 24 paired biopsies (48 total biopsies) from the COMBAT trial. The characteristics of these patients are listed in Table 2-1.

TABLE 2-1
Characteristics of patients with paired biopsies before and on BAT.

| | | | | | | | Change | |
|---|---|---|---|---|---|---|---|---|
| | | | | | | | tumor | |
| | | | | | Baseline | PSA on | volume | Response |
| | Age | | Disease | | PSA | C4D1 | on C4D1 | designa- |
| ID | (yrs) | Prior Therapy | volume* | Site of Biopsy | (ng/ml) | (ng/ml) | (%) | tion |
| 1 | 63 | Abi, Enza, Taxane, | High | Retroperitoneal | 27.8 | 3.2 | −16 | R |
| | | Other AR-targeted | | LN | | | | |
| 3 | 70 | Abi, Enza, Taxane | High | Para-aortic LN | 166.6 | 151.7 | −50 | R |
| 5 | 66 | Enza | High | Paratracheal LN | 34.2 | 70 | NA | NR |
| 6 | 72 | Abi, Enza | Low | Axillary LN | 147.9 | 165.8 | −1 | NR |
| 7 | 68 | Abi, Enza, Taxane, | Low | Retrocaval LN | 249.4 | 123.3 | −30 | R |
| | | Other AR-targeted | | | | | | |
| 8 | 56 | Abi, Taxane | Low | Pelvic LN | 11.9 | 24.6 | −17 | NR |
| 10 | 69 | Abi | Low | Para-aortic LN | 18.2 | 14.6 | −15 | NR |
| 11 | 64 | Abi, Enza, Taxane | High | Axillary LN | 8.0 | 2 | −50 | R |
| 12 | 70 | Abi, Enza | High | Retrocrural LN | 100.0 | 45.1 | −53 | R |
| 13 | 72 | Abi, Enza | High | Retroperineal | 366.3 | 20.6 | −64 | R |
| | | | | LN | | | | |
| 18 | 61 | Abi, Enza | High | Liver | 51.4 | 94.6 | 71 | NR |
| 20 | 72 | Enza | High | Liver | 50.4 | 228.1 | 17 | NR |
| 23 | 86 | Abi, Enza, Taxane, | High | Inguinal LN | 79.2 | 113.80 | −14 | NR |
| | | other AR-targeted | | | | | | |
| 24 | 69 | Abi | Low | Para-aortic LN | 92.9 | 4.6 | −23 | R |
| 29 | 66 | Abi, Enza, Taxane | High | Supraclavicular | 185.2 | 54.2 | −13 | R |
| | | | | LN | | | | |
| 30 | 61 | Abi, Taxane | Low | Para-aortic LN | 16.2 | 62.1 | 30 | NR |
| 31 | 68 | Abi | Low | Para-aortic LN | 29.8 | 16.7 | −13 | NR |
| 32 | 51 | Abi, Enza, Taxane | | Pre- | 168.4 | 109.40 | −13 | NR |
| | | | | sacral/common | | | | |
| | | | | iliac LN | | | | |
| 35 | 75 | Abi, Enza, Taxane | High | Lung | 240.9 | 516.70 | −22 | NR |
| 47 | 57 | Abi | Low | Mediastinal LN | 5.4 | 16 | 0 | NR |
| 49 | 70 | Abi, Enza | Low | Para-aortic LN | 127.1 | 113.3 | 17 | NR |
| 50 | 61 | Abi, Enza, Taxane | Low | Para-aortic LN | 60.2 | 297.035 | −23 | NR |
| 52 | 76 | Abi | Low | Para-aortic LN | 249.8 | 99.65 | −20 | R |
| 53 | 75 | Other AR-targeted | Low | Supraclavicular | 40.0 | 14.7 | 10 | R |
| | | | | LN | | | | |

2.3.1.2 Perform Global Metabolic Profiling on these Samples

We had planned to perform metabolic profiling on these samples by global metabolomics, however there was insufficient tissue obtained from each biopsy to perform metabolic profiling by this approach. Therefore, we performed metabolic profiling by transcriptomic analysis. To do this, frozen sections were cut onto PEN (polyethylene naphthalate) slides (Leica), stained with hematoxylin and LCM was performed at the SKCCC Core facility using a Leica LMC7000 laser capture microdissection system in the SKCCC Cell Imaging Core. Laser captured tissues from regions highly enriched for tumor cells were collected by manual annotations of regions of interest that outlined tumor nests. RNA was isolated using the ALLPREP RNA/DNA extraction protocol (Qiagen Cat. No. 802804). RNA amounts were measured using a Qubit fluorometer (Invitrogen) and quality were measured using an Agilent Bioanalyzer 2100 with the Pico Chips Kit. Purified RNA was provided to the SKCCC Experimental and Computational Genomics Core to carry out their low-input RNA-seq workflow as described previously with some modifications (Freeman et al. 2020). Briefly, quality of total RNA was measured by the Agilent Bioanlayzer to determine RNA integrity (RIN). Samples with starting input between 100 pg-100 ng of total RNA and RIN>7.0 were considered to have sufficient quality to proceed to construction of whole transcriptome sample-barcoded libraries using the Ovation RNA-Seq System V2 according to the manufacturer's protocols (Nugen). Quantification of the libraries was performed by qPCR or by the Agilent Bioanalyzer and equimolar concentrations of each library were pooled together, clustered and sequenced on an Illumina Novaseq 6000 platform, with paired end sequencing. The resulting reads were aligned the human reference genome build hg38 using RSEM (Li and Dewey 2011) with the STAR aligner (Dobin et al. 2013) to obtain posterior mean estimate transcripts per million (pmeTPM) as a measure of gene expression. pmeTPM values for each gene were normalized using upper quantile normalization across samples and log 2 transformed. We were able to perform transcriptomic analysis on 12 paired samples due to limitations of sample quantity and quality.

2.3.1.3 Perform Data Analysis on Metabolic Profiles and Correlate with Clinical Efficacy of BAT

We will compare metabolite concentrations in biopsy samples before and after BAT and perform statistical analysis to determine whether metabolite concentrations change after BAT. We will then correlate these changes with measures of clinical efficacy (PSA₅₀, PFS, OS) to determine whether this is a correlation between changes in metabolites and clinical efficacy of BAT.

GSEA was performed using GSEA 4.2.3 software downloaded from https://www.gsea-msigdb.org (Subramanian et al. 2005; Mootha et al. 2003). Gene sets were downloaded from the Molecular Signatures Database (MSigDB) and included:
HALLMARK_GLYCOLYSIS.v7.5.1,
HALLMARK_OXIDATIVE_PHOSPHORYLATION.v7.5.1,
HALLMARK_FATTY_ACID_METABOLISM.v7.5.1, and
HALLMARK_REACTIVE_OXYGEN_SPECIES_PATHWAY.v7.5.1.

Normalized log 2 transformed read counts by DESeq2 correction of RNA sequencing of micro-dissected tumor samples, stratified by response and/or timepoint, were used for input. Enrichment scores and p values corrected for false discovery were calculated.

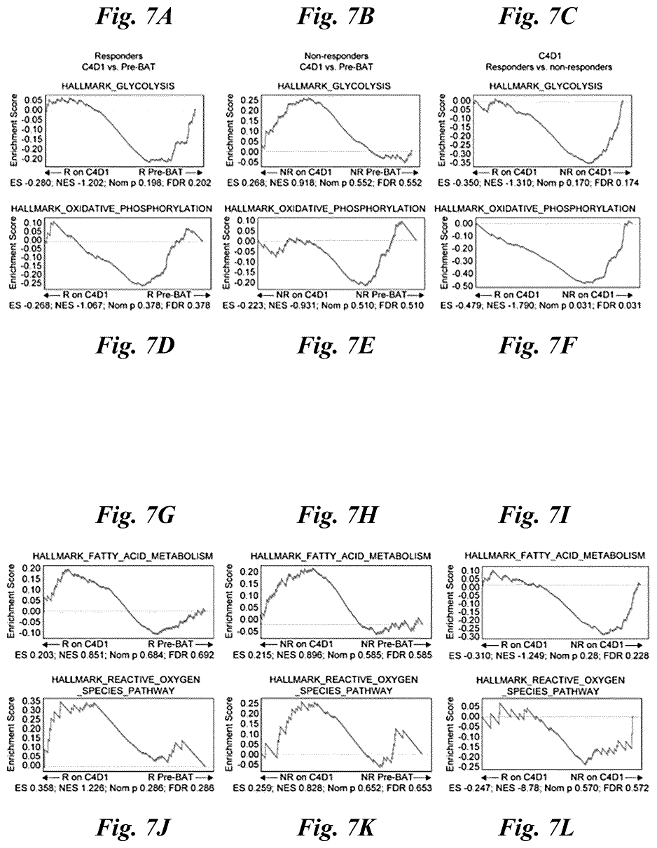

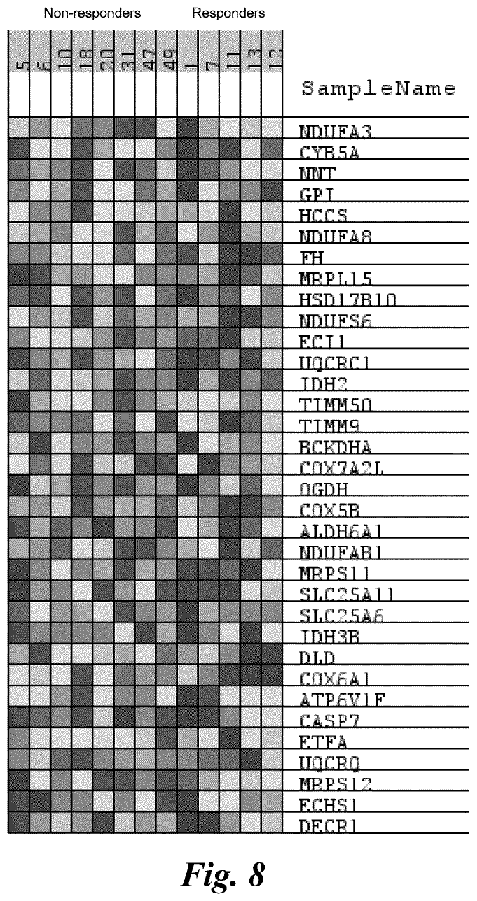

We assessed metabolic changes induced by BAT in responding (R) and non-responding (NR) patients as per Table 2-1. This indicated that BAT resulted in a trend toward decreased expression of genes associated with glycolysis in R but an enrichment of these genes in NR (FIG. 7A-B). On C4D1, there was a trend toward enrichment of these genes in NR compared with NR (FIG. 7C). In contrast, BAT resulted in a trend toward decreased expression of genes associated with oxidative phosphorylation in both R and NR (FIG. 7D-E), and NR had a statistically significant enrichment of these genes compared with R on C4D1 (FIG. 7F). Both R and NR had a trend toward increased expression of genes associated with fatty acid metabolism on BAT (FIG. 7G-H), with NR having a trend toward greater enrichment of these genes on C4D1 than R (FIG. 7I). Similarly, both R and NR had a trend toward increased expression of genes associated with reactive oxygen species signaling (FIG. 7J-K), with NR having a trend toward greater enrichment of these genes on C4D1 than R (FIG. 7L).

The only gene set that was statistically significantly altered was oxidative phosphorylation, which was enriched in NR compared with R on C4D1 (FIG. 7F). To better understand what was driving this difference, we assessed expression of leading-edge genes that were different between NR and R. This assessment showed that mitochondrial complex I (NADH dehydrogenase) transcripts including NDUFA3, NDUFA8, NDUFS6, NDUFAB1, as well as several tricarboxylic acid cycle enzymes including FH and IDH, were reduced in R compared with NR (FIG.). Given that many of these genes are regulated by MYC, this prompted us to study MYC expression. We found that BAT decreased MYC expression in responding patients, which required high baseline activity of the androgen receptor (Sena et al. 2022).

### 2.3.2 Determination of Whether Metabolic Changes Induced by BAT are Required for Androgen-Induced Cell Growth Arrest or Death In Vitro

2.3.2.1 Determination of which Prostate Cancer Cell Lines Best Model In Vivo Results from Section 2.3.1
2.3.2.1.1 Culture LNCaP, VCaP, and LAPC-4 Cell Lines with and without Escalating Doses of R1881 and Measure Cell Proliferation and Cell Death

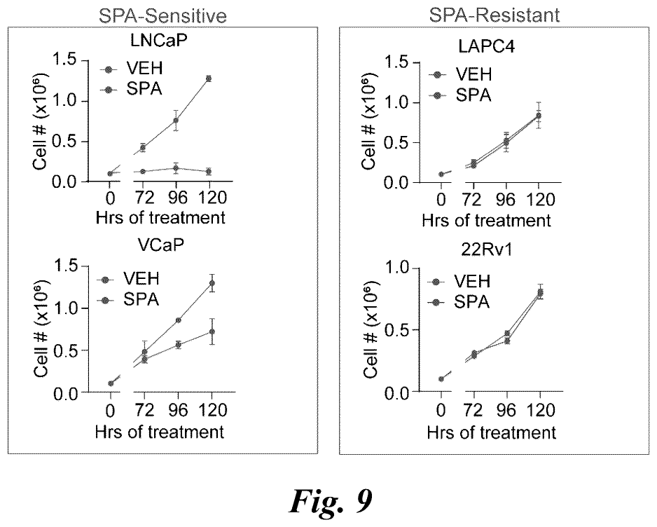

We measured cell proliferation of LNCaP, VCaP, LAPC4, and 22Rv1 cells with increasing duration of vehicle control or R1881 10 nM (i.e., HDA). This observation confirmed prior reports that LNCaP and VCaP are growth-inhibited by HDA, while LAPC4 and 22Rv1 are not (FIG. 9). There was no effect on cell viability in any cell line. We determined that cell line sensitivity to HDA depends on abundance and activity of AR (Sena et al. 2022).

2.3.2.1.2 Culture LNCaP, VCaP, and LAPC-4 Cell Lines with and without Escalating Doses of R1881 and Perform Metabolomics Profiling

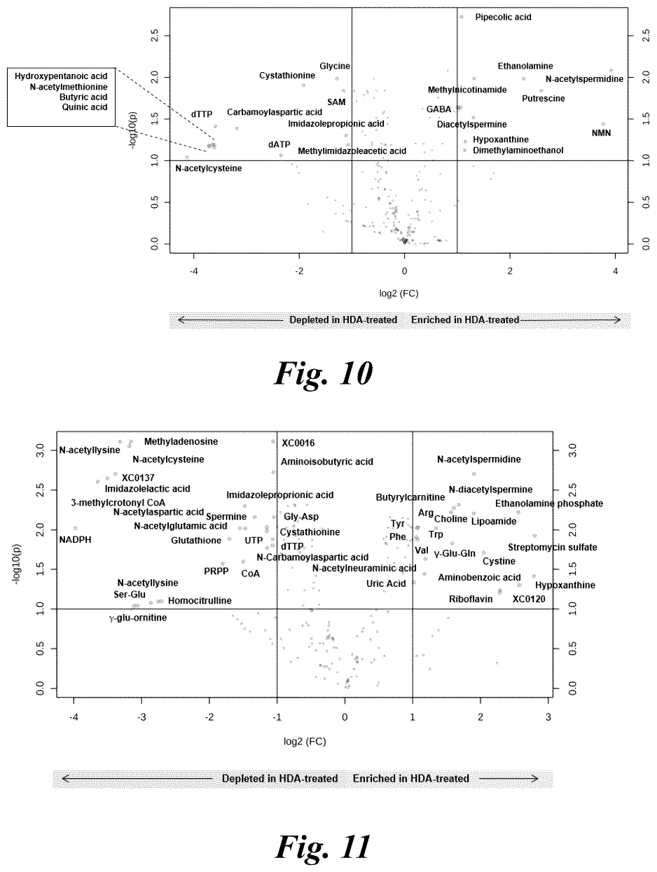

We performed global metabolomics of LNCaP cells after 5-day exposure to HDA (FIG. 10). This measurement indicated that the major changes to metabolite concentration was an increase in polyamines putrescine and spermidine and a decrease in the polyamine precursor S-adenosylmethionine (SAM), and nucleotides.

Next, we performed global metabolomics of LNCaP cells after 26-day exposure to HDA (FIG. 11). The rationale for this experiment was that we determined that with continuous exposure to HDA, LNCaP cells acquired resistance and no longer were growth inhibited. We are interested to understand mechanisms of and novel vulnerabilities induced by resistance to HDA. This analysis showed many differences in metabolites including a persistent increase in polyamine species (N-acetylspermidine and N-diacetylspermine) and a decrease in many other acetylated species including N-acetyllysine, N-acetylcysteine, N-acetylglutaminic acid, N-acetyllysine, and a persistent decrease in some nucleotides and nucleotide precursors including UTP and PRPP.

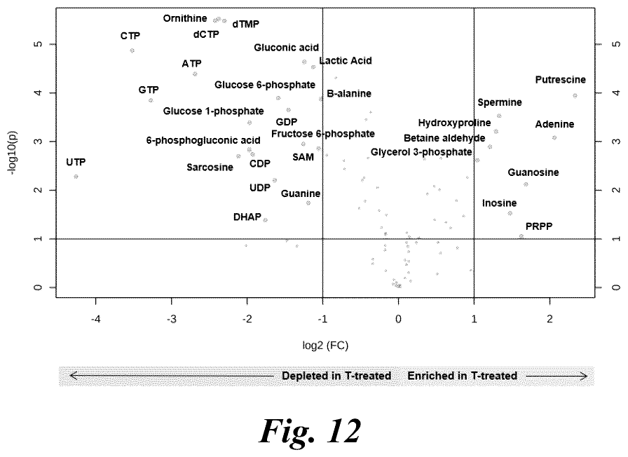

Next, we performed global metabolomics of the patient-derived xenograft (PDX) SKCaP-CR growing in a castrated mouse untreated or treated with testosterone cypionate for 2 weeks (FIG. 12). This PDX is derived from a skin metastasis of a patient with heavily pretreated mCRPC and is growth-inhibited by HDA (Sena et al. 2022). The rationale for this experiment was to determine whether changes to metabolite abundance seen in vitro were similarly altered in vivo. This experiment showed that testosterone increased the polyamines putrescine and spermine and decreased the polyamine precursor SAM and ornithine, as well as multiple nucleotides. This experiment validated that metabolic changes observed in vitro can approximate changes occurring in the in vivo environment.

### 2.3.3 Characterization of Metabolic Changes Induced by Androgen In Vitro

2.3.3.1 Perform Seahorse Experiments to Measure OCR and ECR on Prostate Cancer Cells Stimulated with Androgen

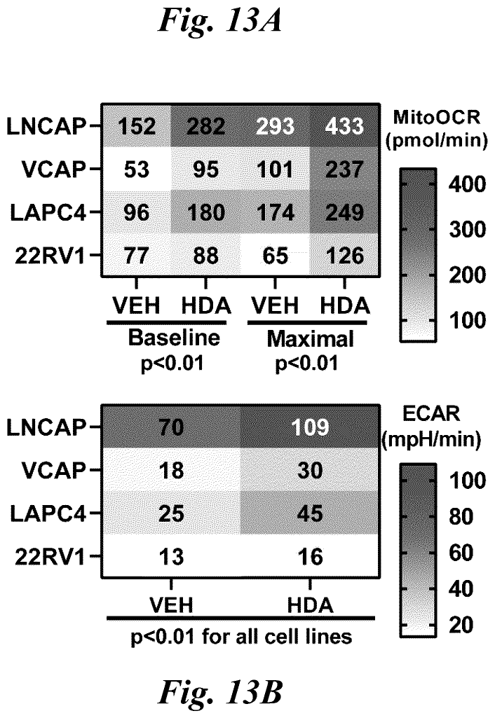

We performed Seahorse experiments to measure extracellular metabolic flux in the prostate cancer cell lines LNCaP, VCaP, 22Rv1, and LAPC4 treated with HDA or vehicle control for 4 days (FIG. 13). This experiment showed that HDA increased baseline and maximal mitochondrial oxygen consumption rate (mitoOCR) in all cell lines (FIG. 13A). Moreover, HDA increased extracellular acidification rate in all 4 cell lines (FIG. 13B).

2.3.3.2 Perform Isotype Trancing Experiments to Measure Metabolic Flux from Glucose Induced by Androgen

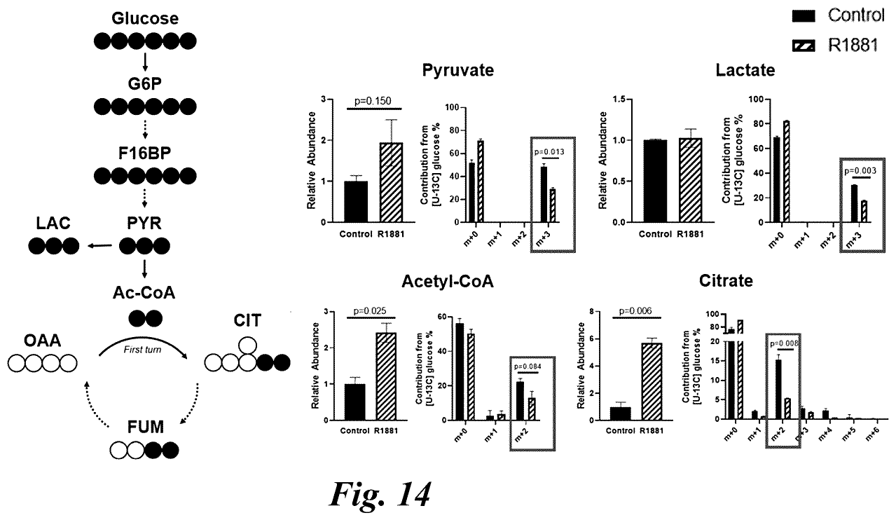

We traced UC13-glucose in LNCaP cells treated with vehicle control or HDA for 4 days and assessed labeling patterns in intracellular metabolites (FIG. 14) and extracellular metabolites (FIG. 15). This experiment showed that while total abundance of pyruvate, acetyl co-A, and citrate were increased by HDA, the contribution from glucose was decreased (FIG. 14). Interestingly total lactate was unchanged, although the contribution of glucose to this pool of lactate was similarly decreased (FIG. 14). When assessing extracellular metabolites, interestingly citrate was markedly increased by HDA, with similar contribution from glucose (FIG. 15). While ECAR is typically considered to be a metric of excretion of lactate, our data suggest that the elevated ECAR due to HDA may actually be due to extraction of citrate.

2.3.3.3 Perform Measurements of Intracellular ROS by roGFP Transfection and Analysis after Androgen Stimulation. Perform Measurements of Lipid and Fatty Acid Concentration by Colorimetric Stain

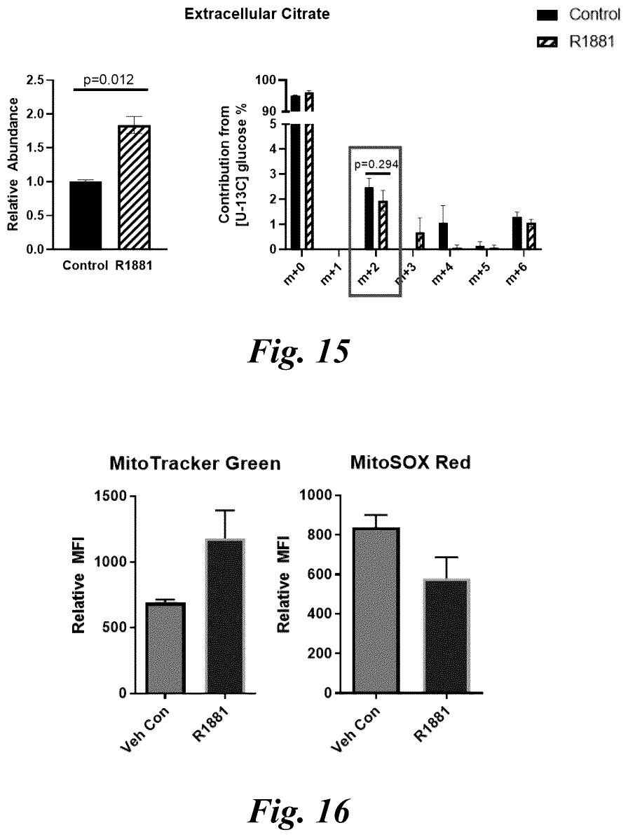

We attempted multiple times to express roGFP in LNCaP cells but we were unable to get good expression of the construct. Therefore, we measured mitochondrial superoxide abundance by mitoSOX red. This experiment showed that contrary to our hypothesis, mitochondrial superoxide abundance decreased in LNCaP cells treated with HDA (FIG. 16). Notably, this occurred in spite of an increased density of mitochondria induced by HDA as assessed by MitoTracker Green (FIG. 16). Altogether our data suggests that after short durations of exposure (4 days) HDA increases mitochondrial density and oxygen consumption. We suspect that longer exposure may subsequently lead to depletion of mitochondria due to depletion of MYC and mitochondrial biogenesis and account for the decreased enrichment of genes associated with oxidative phosphorylation observed in patient samples.

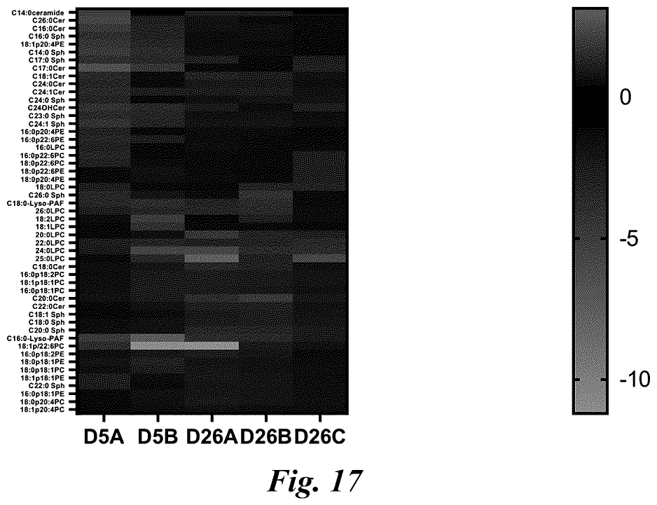

To measure the effect of HDA on lipid abundance and variety, we performed global lipidomics on LNCaP cells treated with HDA or vehicle control for 5 or 26 days (FIG. 17). This showed that some ceramide species such as C26:0Cer, C16:0Cer, and C17:0Cer were increased by HDA on day 5, while some phosphatidylcholine species such as 22: OLPC and 24: OLPC were decreased by HDA on day 5 (FIG. 17). Notably, these changes inverted on day 26, when the cells had acquired resistance to HAD (FIG. 17).

2.3.4 Determination of which Metabolic Pathway is Required for Androgen-Induced Cell Growth Arrest and Death In Vitro
2.3.4.1 Perform Rescue Experiments by Supplementing Cells with Deficient Metabolic Byproducts (for Example DMK or ROS) and Measuring Cell Death and Proliferation after Androgen Stimulation

Our studies indicated that a major change was an increase in polyamines. Therefore we set out to determine whether this metabolic pathway was functionally important for androgen-induced cell growth arrest and death in vitro.

Further studies showed that HDA increased polyamine abundance by stimulating transcription of the rate-limiting enzyme in de novo polyamine synthesis, ornithine decarboxylase (ODC) (FIG. 2). Marked intracellular and extracellular accumulation of putrescine was confirmed by HPLC in the PCa cell line LNCaP treated with the synthetic androgen R1881 at the supraphysiological dose of 10 nM (FIG. 2a). Moreover, BAT increased plasma putrescine abundance in patients with CRPC (FIG. 2b). This increase in putrescine abundance likely occurs due to enhanced expression of ODC, as SPA increased ODC protein and activity in PCa cell lines (FIG. 2c-FIG. 2d). Beyond ODC, we suspect that multiple enzymes that promote polyamine synthesis and excretion are stimulated by AR activation, as SSAT activity was also significantly increased by SPA (FIG. 2c). Moreover, we observed that ODC1, as well as genes coding for several polyamine metabolism enzymes, was significantly downregulated in advanced PCa tumors after 22 weeks of androgen deprivation therapy (ADT) in the Rajan et. al dataset (Rajan et al. 2014) (FIG. 2e), and ODC1 expression positively correlated with AR activity in metastatic PCa tumors in the SU2C/PCF dataset (Abida et al. 2019) (FIG. 2f). Together these data indicate that AR activation upregulates ODC to increase intracellular and excreted polyamines. In future research we will seek to define mechanisms by which AR regulates ODC.

To assess the functional significance of stimulated polyamine synthesis by SPA, we tested the highly specific and clinically-utilized inhibitor of ODC, difluoromethylornithine (DFMO). To assess additive antitumor effect through combination with DFMO, we treated LNCaP and VCaP PCa cells with a low dose of R1881 (0.5 nM, inhibits clonal survival by approximately 15%). DFMO inhibited clonal survival to a greater extent in R1881-treated cells than vehicle control-treated cells (FIG. 3a-FIG. 3b). Growth inhibition could be rescued by supplementing with exogenous putrescine (FIG. 3a-FIG. 3b), indicating that reduction of putrescine synthesis by DFMO increases growth suppression of CRPC by SPA. To assess whether combined therapy with SPA and DFMO has efficacy in vivo, we tested these agents in the subcutaneous castration-resistant syngeneic (immune-competent) mouse model, MYC-CaP-CR (Ellis et al. 2012). This experiment indicated that combination treatment with SPA and DFMO was superior to either agent alone in reducing the rate of tumor growth in vivo (FIG. 3c). These data suggest that SPA-stimulated synthesis of polyamines is protective for CRPC, and inhibiting this pathway using DFMO has potential to improve the efficacy of BAT. Future research will address the novel hypothesis that regulation of ODC by AR modulates PCa cell fate and tumor immunity following hormonal manipulation. Without wishing to be bound to any one particular theory, it is thought that ODC activation by AR and SPA drives resistance to SPA and tumor immune tolerance, while ODC inactivation by AR inhibition drives efficacy of AR inhibition. These studies will provide insight into mechanisms of efficacy and resistance to AR-targeted therapies, which will enable development of improved therapeutic strategies for patients with PCa.

Without wishing to be bound to any one particular theory, it is thought that one possible contributor to why polyamines can drive resistance is that polyamines can drive immunosuppression. PCa is known to create a highly immunosuppressive microenvironment and is poorly responsive to T cell checkpoint inhibitors. Future research will assess the novel hypothesis that the immunosuppressive microenvironment of PCa is in part driven by abundant polyamines, driven by AR-mediated stimulation of ODC.

To begin to assess this possibility, we characterized the immune infiltrate of MYC-CaP-CR tumors growing in castrated FVB mice treated with empty pellet, testosterone pellet, DFMO, or testosterone and DFMO. As per FIG. 3, combination therapy with testosterone and DFMO resulted in greater growth inhibition than empty pellet or either therapy alone. High dimensional flow cytometry using a panel of 30 markers of immune cell lineage and functional phenotype showed marked differences in the phenotype of tumor-infiltrating T cells between treatment groups (FIG. 6a) driven primarily by expression of activation markers including perforin, CD39, CD44, and LAG3 (FIG. 6b). SPA+DFMO treatment enhanced the percentage of CD8 T cells expressing granzyme and perforin (FIG. 6c), indicating enhanced cytotoxic function by combination treatment. Notable differences in myeloid cell phenotype were also observed (FIG. 6d), driven primarily by altered macrophage markers (FIG. 6e), with reduction of the M2 macrophage marker CD206 and enhancement of the M1 macrophage marker CD86 in combination treatment tumors (FIG. 6f). Together this experiment suggests that SPA+DFMO enhances inflammatory phenotypes of tumor-infiltrating immune cells.

### Example 3

Repeat Sequential DFMO and High Dose Testosterone in Sequence with Enzalutamide in Asymptomatic Patients with Metastatic Castration-Resistant Prostate Cancer

### 3.1 Overview

This Example describes a Phase II Study of Repeat Sequential DFMO and High Dose Testosterone in Sequence with Enzalutamide in Asymptomatic Patients with Metastatic Castration-Resistant Prostate Cancer: The APEX Trial (Androgen and Polyamine Elimination alternating with Xtandi). The primary objective of the study is to determine if treatment with the combination of difluoromethylornithine (DFMO or eflornithine) and high dose testosterone will improve the prostate-specific antigen (PSA) response rate in patients with metastatic castrate-resistant prostate cancer (mCRPC) compared with historical controls. The secondary objective is to determine if repeat treatment with DFMO and high dose testosterone in sequence with enzalutamide will improve progression free survival (PFS) compared to historical controls.

### 3.2 Study Design

Asymptomatic patients with mCRPC without pain due to prostate cancer will be treated on an open label study to evaluate effectiveness of sequential treatment with the combination of DFMO and high dose testosterone in sequence with enzalutamide to improve primary and secondary outcomes.

### 3.2.1 Treatment Plan

Eligible patients are those with mCRPC who have progressive disease after treatment with Abiraterone (Abi) used as treatment for castration-sensitive or castration-resistant disease. Patients will continue on ADT with LHRH agonist (i.e., Zoladex, Trelstar, Eligard, or Lupron) or LHRH antagonist (Degarclix or Relugolix) if not surgically castrated throughout the duration of the study to inhibit endogenous testosterone production.

In one representative embodiment, one cycle of treatment will be 119 days and will involve:
1. 7 days of DFMO at a dose of 1000 mg PO BID (D1-D7), followed by
2. 56 days of combined testosterone and DEMO (testosterone cypionate 400 mg IM on D8 and D36 with continued DFMO 1000 mg PO BID) (D8-D63), followed by
3. 56 days of enzalutamide (enzalutamide 160 mg PO daily) (D64-D119).

Patients will receive repeat cycles of treatment until clinical or radiographic progression or toxicity requiring drug cessation. Patients will have symptoms assessed on Day 1, Day 36, and Day 64 of each cycle. Bone and CT scans to evaluate treatment response will be performed on Day 64 of cycle 1 and on Day 1 of subsequent cycles (i.e., every 8 weeks for the first 2 scans, then every 16 weeks thereafter). Radiographic progression will be defined by RECIST criteria (i.e., greater than 20% increase in sum of target lesions) on CT scans and PCWG3 criteria (i.e., greater than or equal to 2 new bone lesions) on bone scans. Patients with clinical progression due to pain flare after first two injections of testosterone can remain on study. If pain persists after first treatment with enzalutamide, patients will stop treatment and come off study. If pain resolves on enzalutamide, but returns with next or subsequent cycles of testosterone, patients will stop treatment and come off study.

### 3.2.2 Study Population

Patients with CRPC with progressive disease (radiographically and/or biochemically) who have been treated with continuous castrating therapy and Abi. Twenty slots are reserved for patients with disease amenable to biopsy. A representative number of patients is about 50 patients.

Patient inclusion criteria include, but are not limited to: (1) ECOG Performance status ≤2; (2) age ≥18 years; (3) histologically-confirmed adenocarcinoma of the prostate; (4) treated with continuous androgen ablative therapy (either surgical castration or LHRH agonist/antagonist); (5) documented castrate level of serum testosterone (<50 ng/dl); (6) metastatic disease radiographically documented by CT or bone scan; (7) must have had disease progression while on abiraterone acetate based on: (a) PSA progression defined as an increase in PSA, as determined by two separate measurements taken at least one week apart, and/or (b) radiographic disease progression, based on RECIST 1.1 in patients with measurable soft tissue lesions or PCWG3 for patients with bone disease; (8) screening PSA must be ≥1.0 ng/ml; (9) patients with soft tissue lesion amenable to biopsy must agree to biopsy collection pre-treatment and at a defined point on treatment to perform tumor tissue analysis; (10) prior treatment with Provenge vaccine, 223Radium (Xofigo), PARP inhibitors, taxane chemotherapy, Lu-PSMA, antiandrogens (including enzalutamide, darolutamide, and apalutamide), and radiation is allowed if >4 weeks from last dose; (11) prior treatment with BAT is allowed if the patient has progressed on an AR-axis inhibitor (i.e., abiraterone or antiandrogen) since BAT treatment; (12) patients must be withdrawn from abiraterone for ≥ 2 weeks; (12) attempts must be made to wean patients off prednisone prior to starting therapy. Patients who cannot be weaned due to symptoms may continue on lowest dose of prednisone achieved during weaning period; (13) acceptable liver function: (a) Bilirubin<2.5 times institutional upper limit of normal (ULN) and (b) AST (SGOT) and ALT (SGPT)<2.5 times ULN; (14) acceptable renal function: GFR of 50 mL/min/1.73 m² or higher. GFR will be estimated by the 2021 chronic kidney disease epidemiology (CKD-EPI) creatinine equation (Inker L A, Eneanya N D, Coresh J, et al. Chronic Kidney Disease Epidemiology Collaboration. New Creatinine- and Cystatin C-Based Equations to Estimate GFR without Race. N Engl J Med 2021; 385:1737) using the online calculator found on UpToDate (uptodate.com/contents/calculator-glomerular-filtration-rate-gfr-by-ckd-epi-equation-in-adults-conventional-and-si-units?search=gfr&topicRef=2359&source=see_link); (15) acceptable hematologic status: (a) absolute neutrophil count (ANC)≥1500 cells/mm³ (1.5×10⁹/L); (b) platelet count ≥100,000 platelet/mm³ (100×10⁹/L); (c) hemoglobin≥8 g/dL; (16) ability to understand and willingness to sign a written informed consent document; (17) sexually active participants with female partners of childbearing potential are eligible to participate if they agree to follow one of the following methods of contraception consistently, starting from screening, during the study and for at least 3 months after the last dose of DFMO and/or enzalutamide: (a) are abstinent from penile-vaginal intercourse as their usual and preferred lifestyle (abstinent on a long-term and persistent basis) and agree to remain abstinent; (b) are sterilized (with the appropriate post-vasectomy documentation of the absence of sperm in the ejaculate); (c) agree to use a male condom and have their partner use contraceptive method with a failure rate of <1% per year as described below when having penile-vaginal intercourse with a woman of childbearing potential who is not currently pregnant, and who agrees to the use of a condom by her partner; (18) in addition, participants must refrain from donating sperm starting from Screening, during the study and for at least 3 months after the last dose of DFMO and/or enzalutamide; and (19) sexually active participants with a pregnant or breastfeeding partner must agree to remain abstinent from penile-vaginal intercourse, or use a male condom during each episode of penile penetration during the study.

Exclusion criteria include, but are not limited to: (1) pain due to metastatic prostate cancer requiring treatment intervention with opioid pain medication; (2) ECOG Performance status ≥3; (3) requirement for urinary self-catheterization for voiding due to obstruction secondary to prostatic enlargement well documented to be due to prostate cancer or benign prostatic hyperplasia (BPH). Patients with indwelling Foley or suprapubic catheter for obstructive symptoms are eligible; (4) evidence of disease in sites or extent that, in the opinion of the investigator, would put the patient at risk from therapy with testosterone (e.g., femoral metastases with concern over fracture risk, severe and extensive spinal metastases with concern for spinal cord compression, extensive liver metastases); (5) active uncontrolled infection. Patients with a history of HIV/AIDS may be eligible if CD4+ T cell counts are ≥350 cell/μL, they have had no opportunistic infection within the past 12 months, they have been on established antiretroviral therapy (ART) for at least four weeks, the HIV viral load is less than 400 copies/mL prior to enrollment, and there is no significant drug-drug interaction with ART and the study drugs. Patients with chronic HBV infection with active disease who meet criteria for anti-HBV therapy are eligible if they are on a suppressive antiviral therapy prior to enrollment and there is no drug-drug interaction with the study drugs. Patients with a history of HCV infection are eligible if they have completed curative antiviral treatment and the HCV viral load is below the limit of quantification; (6) any condition or mental impairment that may compromise the ability to give informed consent, patient's safety or compliance with study requirements as determined by the investigator; (7) patients receiving anticoagulation therapy with warfarin, rivaroxaban, or apixaban are not eligible for study. [Patients on enoxaparin are eligible for study. Patients on warfarin, rivaroxaban, or apixaban, who can be transitioned to enoxaparin prior to starting study treatments will be eligible]; (8) patients are excluded with prior history of a thromboembolic event within the last 12 months that are not being treated with systemic anticoagulation; (9) hematocrit>51%, untreated severe obstructive sleep apnea, uncontrolled or poorly controlled heart failure [per Endocrine Society Clinical Practice Guidelines (34)]; (10) patients allergic to sesame seed oil or cottonseed oil are excluded; (11) major surgery (i.e., requiring general anesthesia) within 3 weeks before screening, or has not fully recovered from prior surgery (i.e., unhealed wound). Note: subjects with planned surgical procedures to be conducted under local anesthesia may participate; (11) subjects with significant hearing loss defined as hearing loss that affects everyday life and/or for which a hearing aid is required; and (12) patients with history of seizure or any condition that may predispose to seizure (e.g., prior cortical stroke or significant brain trauma, brain arteriovenous malformation).

### 3.2.3 Study Endpoints

3.2.3.1 Primary Endpoint: PSA response rate (>50% PSA decline from baseline) by C1D64.
3.2.3.2 Secondary Endpoints: (1) Progression-free survival (PFS): time to radiographic or clinical progression or death [PCWG3 definition]; (2) PSA response rate (>50% PSA decline from baseline) at any point on trial; (3) Incidence and severity of adverse events and serious adverse events graded according to CTCAE v5.0; (4) PSA progression-free survival (PSA-PFS) [PCWG3 criteria]; (5) objective response rate in those with measurable disease; (6) change in pain score on the short-form McGill Pain Questionnaire (SF-MPQ); and (7) overall survival.

### 3.2.4 Statistical Plan

3.2.4.1 Primary objective and primary endpoint: The primary objective of the study is to assess the PSA response on C1D64, after the first course of DFMO+T combination therapy. The primary efficacy endpoint is the PSAso response, defined as >50% PSA decline from baseline by C1D64.
3.2.4.2 Analysis of primary endpoint: PSAso response rate will be estimated as the proportion of patients who has >50% PSA decline from baseline by C1D64, along with 95% exact confidence interval. A patient will be considered evaluable if he receives at least one dose of T in combination with DFMO.
3.2.4.3 Sample size and power considerations: The PSA₅₀ response rate to 64 days of BAT monotherapy is approximately 25% in patients with mCRPC (1-4). We expect that the addition of DFMO to BAT will increase the PSAso response rate on C1D64 to 40%. The sample size is calculated to detect an improved PSA₅₀ response rate from 25% to 40% by C1D64. A two-stage design is planned. A total of 24 patients will be entered in the first stage. If <=4 subjects have PSA₅₀ response, the treatment will be terminated and we will conclude the regimen is ineffective. If >=5 subjects respond, then additional 22 patients will be studied. If a total of 15 or fewer subjects have PSAso response by C1D64 in stage one and two combined, we consider the regimen ineffective. If a total of 16 or more respond, we conclude the regimen is promising and warrant further study. The maximum sample size will be 46 evaluable patients. This design provides 81% power to detect a difference of 15% of PSA₅₀ response rate with a type I error of 0.09 (target type I error of 0.1). Accounting for an approximate 10% drop-out rate before evaluation, the trial will enroll 50 patients. Based on our prior experience with BAT studies in this patient population, we project to enroll 2-3 patients/month, allowing for enrollment completion over 16-25 months. The power calculation is performed using PASS 2022 software.
3.2.4.4 Analysis of secondary endpoints: Secondary efficacy endpoints (PSA-PFS, PFS and OS) will each be summarized using the Kaplan-Meier approach. Objective response rate will be estimated. Toxicity and adverse events will be tabulated by type and grade according to CTCAE v5.0.
3.2.4.5 Study Schema:

### 3.3 Background

### 3.3.1 Overview and Rationale

Prostate cancer is uniformly lethal once it has escaped the confines of the prostate gland, resulting in the death of over approximately 30,000 American patients each year (5). Androgen ablation therapy has remained the standard of care for patients with recurrent/metastatic cancer since its discovery by Charles Huggins in the 1940s (6). While androgen ablation therapy provides significant palliative benefit, all patients undergoing androgen ablation eventually relapse and no longer respond to androgen ablation no matter how completely given (7,8).

This observation led to the labeling of patients progressing on androgen ablative therapies as having “androgen independent” or “hormone refractory” prostate cancer. New findings, however, have demonstrated that the majority of prostate cancer specimens from androgen ablated patients continue to express the androgen receptor (AR) often at higher levels (9,10). In addition, variants of AR that do not bind to ligand also are upregulated in androgen deprived prostate cancer cells. Prostate cancer cells from these castration refractory patients continue to express AR-regulated genes, such as PSA. This observation has resulted in a reclassification of “hormone refractory” disease as “Castration Resistant Prostate Cancer” (CRPC) and has opened up new avenues of research into the function of the AR in the androgen deprived state. These findings suggest that “castration-resistant” prostate cancer may continue to survive through aberrant AR signaling. Studies have demonstrated adaptation to chronic androgen deprivation through several mechanisms including marked upregulation of the full length AR, AR gene amplification, and expression of AR splice variants lacking the ligand binding domain (9-12). This observation has led to a renewed interest in the AR axis as a therapeutic target. On this basis, enzalutamide, a new antiandrogen, and abiraterone acetate, a CYP17 androgen synthesis inhibitor, have recently been approved as second line therapy for prostate cancer on the basis of modest observed improvements in overall survival vs. placebo in randomized phase III trials (13-16).

A major mechanism for the development of CRPC following chronic exposure to androgen ablative therapies is the ability of prostate cancer cells to adapt to the lack of ligand by marked upregulation of the full-length AR and AR splice variants lacking the ligand binding domain. AR gene amplification also is commonly seen in samples from patients on chronic androgen deprivation. Laboratory studies have documented this upregulation of AR. These studies have demonstrated that this upregulation of AR may be responsible for the resistance to antiandrogens. In these studies, re-exposure of androgen starved prostate cancer cells readapt upon exposure to androgen by lowering AR expression. This lowered AR expression now re-sensitizes these cells to androgen ablative therapies, such as antiandrogens (11,12).

In this background of renewed interest in blocking the AR, there has been the paradoxical observation that the growth of both androgen sensitive and androgen resistant prostate cancer cell lines is inhibited by the addition of testosterone or other synthetic androgens to the media (12, 17-19). Typically, in vitro data in human prostate cancer cell lines demonstrate a biphasic response to androgens, with very low levels producing modest growth stimulation and expression of prostate tissue differentiation markers, such as PSA, while higher levels of androgen in the media suppress growth and PSA production (12, 17-19). High levels in this case can be as low as picomolar concentrations suggesting that these “androgen ablation resistant” cells are exquisitely sensitive to androgens. These in vitro studies are supported by animal studies that have demonstrated that androgen receptor positive human prostate cancer cells selected to grow in castrated animals upregulate androgen receptor levels. Similar to the in vitro response, in these models, systemic testosterone administration produces significant growth inhibition, whereas antiandrogens such as bicalutamide promote prostate cancer growth.

Until recently, the mechanisms underlying this paradoxical response have been unknown. Recent data from our group and others, however, have described several possible mechanisms for this effect of androgens on the growth of CRPC cells. The androgen receptor has been shown to be a licensing factor involved in DNA relicensing during progression through the cell cycle (16,18). AR is degraded as the prostate cancer cell goes through cycle. We have demonstrated that the high levels of AR seen in CRPC cells do not get sufficiently degraded in the presence of high dose androgen due to androgen stabilization of the AR. Thus, under these conditions, AR remains bound to origins of replication preventing the cell from progressing through subsequent cell cycles and ultimately resulting in cell death. In addition, it has been demonstrated that replenishment of androgen to androgen starved prostate cancer cells rapidly produces significant double strand DNA breakage that can result in inhibition of growth, inhibition of protein synthesis, growth and loss of clonogenic survival (20). Finally, androgen starved cells upregulate constitutively active AR splice variants that cannot bind androgen due to loss of the ligand binding domain (21,22). CRPC cells may rely on these truncated AR variants for survival under low ligand conditions. It has been shown, however, that when androgen starved CRPC cells are given high dose androgen, expression of these variants is rapidly downregulated to often undetectable levels (22,23).

On the basis of these observations, without wishing to be bound to any one particular theory, it is thought that significant clinical response can be achieved in patients with long standing castration resistant prostate cancer by rapidly cycling from the polar extremes of high dose to castrate serum levels of androgen. We have termed this approach Bipolar Androgen Therapy (BAT) (24). By pursuing this treatment strategy, high AR-expressing CRPC cells will be sensitive to killing by high dose levels of testosterone according to mechanisms described above. Those cells that try to adapt to high androgen by dropping AR expression to low levels will then become sensitive to killing when testosterone levels are lowered to near castrate levels.

### 3.3.2 Androgen Ablative Therapy for Prostate Cancer

In the current treatment approach for CRPC, patients with recurrent or metastatic prostate cancer are initially treated with LHRH agonists, which typically result in castrate levels of serum T (i.e. <50 ng/dL) within 2-4 weeks post initiation of therapy (25). The catastrophic loss of androgen as their major growth and survival factor results in the death of the majority of prostate cancer cells. On this basis, the majority (approximately 90%) of patients have an initial beneficial palliative response to ADT. Relapse, however, occurs in all patients treated with ADT. Over time prostate cancer cells that survive the initial acute drop in serum androgen adapt to the chronic low androgen conditions by upregulating AR through overexpression, gene amplification and expression of truncated, transcriptionally active AR splice variants (AR-V) that lack the ligand binding domain. The first clinical manifestation of this adaptive increase in AR signaling is the renewed production of PSA. At this point the patient is considered to have CRPC. Typically, this patient would continue on ADT and begin second-line hormonal therapies. This approach is based on the concept that a sufficient amount of androgen is produced by the adrenal glands and perhaps by the prostate cancer cells themselves to support the growth of the surviving adapted CRPC cells. Thus, second line hormonal therapies were developed that either competitively inhibit androgen binding to AR (e.g., anti-androgens, including flutamide, bicalutamide, and nilutamide) or inhibit adrenal androgen synthesis (e.g., ketoconazole) (25). While clinical benefit was demonstrated, until recently, the effect of second-line therapy on survival was unknown due to lack of appropriately powered studies. Enzalutamide and abiraterone, however, both recently received FDA-approval for use in metastatic CRPC based on a modest survival benefit observed in large, randomized studies, Table 3-1 (13-16).

The current treatment approach for CRPC is to continue chronic LHRH agonist therapy despite progression and administer “second line” hormone therapy. Based on a demonstrated survival benefit, Abiraterone is emerging as the preferred initial second line therapy. Before the availability of Enzalutamide, standard therapy in patients with progression on Abiraterone would be to give docetaxel chemotherapy. Currently, Enzalutamide is FDA-approved for use in patients post docetaxel based on Phase III results showing approximately 5 months improvement in survival in the post-docetaxel setting, Table 3-1. Enzalutamide, however, is frequently being administered to patients prior to docetaxel if insurance clearance can be obtained. It also is expected that enzalutamide, like abiraterone, will be approved for use in the pre-chemotherapy setting based on positive results from the PREVAIL study that showed an improvement in median overall survival that was estimated at 32.4 months in the enzalutamide group and 30.2 months in the placebo group (hazard ratio, 0.71; 95% CI, 0.60 to 0.84; P<0.001) (16).

Thus, based on the result of these studies, the ease of administration of these oral agents, and the possibility of delaying the need for chemotherapy, the evolving treatment paradigm will likely involve the sequential addition of abiraterone and enzalutamide to LHRH agonist-based ADT in patients with ADT progression. Several small studies, however, have demonstrated that sequential use these agents in the post-chemotherapy setting is associated with significant reduction in time to radiographic progression to <5 months, decreased PSA response, and objective response suggesting cross-resistance between these agents, Table 3-1 (25-28). Limited information is available on progression free survival with enzalutamide post-abiraterone in the pre-chemotherapy setting, but data on small number of patients indicates median rPFS≤6 months (29,30). The mechanisms underlying this reduced response rate are likely multi-factorial and may include continued adaptive increase in AR expression, increased expression of ligand independent AR variants in resistant cells, and emergence of AR mutations that may affect enzalutamide binding.

TABLE 3-1
Results from sequential treatment with Enzalutamide after Abiraterone in CRPC.

| | | >50% decline | | | |
|---|---|---|---|---|---|
| | | PSA | PSA | TIP | Objective |
| | | (%) | PFS | (m) | Response (%) |
| Enzalutamide Phase II (PREVAIL) | 872 | 78 | 11.2 | 20 | S9 |
| Enzalutamide post Abiraterone | 35 | 28,6 | NA | 4.9 | 2.9 |
| | 39 | 12.8 | NA | 2.8 | 4,3 |
| | 29 | 18 | 4.0 | NA | NA |
| | 2 | 29 | 3.0 | NA | |
| | 30 | 34 | 4.1 | 4.7 | NA |
| | 47 | 25.5 | NA | 6.6 | |
| | 214 | 27 | 5.7 | 8.1 | NA |

3.3.3 Clinical Experience with Testosterone in Prostate Cancer

Up until recently, there had been very limited clinical experience in the PSA-era treating CRPC patients with testosterone. Brendler et al. at the Brady Urological Institute reported in the Archives of Surgery in 1949 on the use of parenteral testosterone in several patients with advanced CRPC. They observed considerable improvement in several patients that included decreased pain, decreased prostate size and decreases in acid and alkaline phosphatase. In a second study, Prout and Brewer reported in Cancer in 1967 on the treatment of patients who had been either untreated or recently castrated or long-term castrates with parenteral testosterone (31). Five relapsed patients in the long-term castrate group received testosterone for at least one month and 4 of 5 had subjective improvement. Five remaining patients in the long-term castrate group received testosterone for 1 to 19 days, with each progressing and subsequently coming off therapy. Acid phosphatase declined in 2/5 patients receiving a longer course of testosterone. Remarkably, one man in this group admitted to hospital with severe back pain, weakness and anorexia had a 10-month response with complete cessation of pain, excellent appetite and weight gain with decrease in acid phosphatase from 50 to 5 units.

In contrast, a number of studies during the 1960-70s evaluated the use of T-priming in combination with ³²P-sodium phosphate to treat patients with CRPC and severe pain due to widespread bony metastases (32,33). In these studies, initial T-priming using a variety of parenteral dosing regimens was associated with transient increase in bone pain during the first week followed by excellent pain palliation following administration of ³²P. Similar results were observed in studies led by Manni, who evaluated T-priming with chemotherapy in the 1980's (34). These studies also were conducted in patients with CRPC and pain due to widely metastatic disease. In these studies, increased bone pain also was observed in patients upon initial treatment with oral androgens. The increased pain in these studies typically occurred within days of T administration. Thus, given this time frame, it is likely the increased pain was due to T-stimulation of inflammation/cytokine release within sites of bone metastases rather than a direct effect on tumor growth. Such rapid change also is seen in patients with bone pain upon initially starting ADT. Marked improvement in pain after ADT often occurs within hours of treatment, an effect not due to tumor death but rather a rapid change in expression of pain-promoting gene products.

More recently, two Phase I studies were reported describing the results of the use of transdermal T as therapy for patients with CRPC who had minimal to moderate disease burden and no pain due to prostate cancer. In the first study, Szmulewitz et al. evaluated the effect of increasing doses of transdermal T in 15 patients with early CRPC (rising PSA and minimal bone disease) (34). Five patients each were treated with 2.5, 5.0 or 7.5 mg/day of transdermal T, which brought the median concentration of T from castrate to 305, 308 and 297 ng/dl respectively. In this study, no grade 3 or 4 toxicities were observed with the exception of one man who was taken off study at week 53 for grade 4 cardiac toxicity. Only one patient had symptomatic progression and three patients (20%) had a decrease in PSA (largest was 43%). Patients treated at the highest T dose had a prolonged time to progression that did not reach statistical significance most likely due to the small cohort size. In the second study, Morris et al. evaluated the effect of transdermal T at a dose of 7.5 mg/day administered for 1 week (n=3), 1 month (n=3) or until disease progression (n=6) in 12 patients with CRPC (35). They observed no grade 3 or 4 toxicities and no pain flares. Eugonadal serum T levels were reported for this study. No objective responses were observed. Four patients had at least 20% declines and one achieved a >50% PSA decline.

Neither of these Phase I studies achieved the high dose levels of serum T that can be reached with FDA-approved doses of T administered as an intramuscular depot (36). The levels of serum T achieved, however, were in the high-end of the eugonadal range. Remarkably, although the studies were considered “negative” from the standpoint of disease response, in both studies the administration of parenteral T to patients with CRPC was very well-tolerated and did not result in significant worsening of disease or symptoms, including pain flares. While only one patient out of 27 from the combined studies had a reported >50% decline in PSA, smaller PSA declines were observed in a few of the patients on these two studies with a trend toward a dose-responsive effect, suggesting a potential for therapeutic benefit in some patients (34,35).

Based on the preclinical results and potential mechanisms for growth inhibition that include androgen-induced double strand breaks and stabilization of AR preventing relicensing, we have conducted a pilot study evaluating the efficacy and safety of pharmacologic doses of testosterone to produce high dose T levels in conjunction with oral etoposide in chronically castrated patients with rising PSA and CRPC (37). Patients who had been continuously castrate for more than one year with minimal metastatic disease burden (≤5 total bone metastases and ≤10 total sites of metastases) and/or rising PSA were eligible. To achieve rapid cycling between high dose and near castrate serum T (i.e., BAT) patients received intramuscular injection of 400 mg testosterone cypionate every 28 days. For the first 3 cycles of therapy patients received BAT plus oral etoposide 100 mg po/day days 1-14 of a 28-day cycle. After 3 cycles PSA response and objective response were assessed. Those patients with a PSA that was declining from peak levels and no objective evidence of disease progression or worsening pain were continued on therapy. Given the toxicity associated with etoposide and the lack of clinical response in an earlier trial (38), patients who responded after 3 cycles of testosterone plus etoposide were continued on testosterone alone based on protocol amendment.

Seven of fourteen patients had a decline in PSA from baseline value. An eighth patient progressing after testosterone treatment for 6 months had a decline in PSA upon reaching castrate level testosterone. Non-responders came off of trial after 3 cycles due to PSA progression. For the seven patients that had a PSA decline, the median time to PSA progression was 221 days (range, 95 to 454 days).

The dose of 400 mg T cypionate produces high dose levels >1500 ng/dl within 2 days post injection. At baseline, ten subjects had RECIST-evaluable soft tissue metastases. Of these patients, two (20%) had progressive disease (PD), three (30%) had stable disease after a median follow up of 91 days (range, 87 to 92 days), four (40%) had partial responses (PRs) and one (10%) had a complete response (CR). None of the 14 patients completing 3 months of therapy developed new bone metastases. One patient with >50% decrease in PSA had intensification of an isolated tibial metastases on bone scan and was removed from study despite decline in PSA levels. No other patient developed worsening pain on study.

### 3.4 Adverse Events

The majority of adverse events (AEs) occurred during the initial phase of treatment and were largely consistent with known side effects of etoposide. Initial-phase side effects were mostly low grade (i.e., ≤grade 2) and included: nausea (N=10), fatigue (N=9), alopecia (N=9), edema (N=8) and neutropenia (N=3). Two patients had grade 3 asymptomatic, subsegmental pulmonary embolism. Two subjects did not complete the initial treatment phase, one individual was taken off study after developing grade 2 priapism and a second individual expired due to pneumonia/neutropenic sepsis. AEs occurring during the BAT monotherapy phase of the trial were rare and low grade. Only four subjects experienced an AE during this phase, and all but three AEs were grade 1. Grade 2 events included alopecia and an elevated creatinine in one subject and grade 2 nausea in a separate subject.

None of the 14 patients developed new pain, skeletal events or urinary obstruction due to prostate cancer. Although quality of life was not formally evaluated in the study, most subjects reported enhanced well-being and increased functional activity. Patients with intact sexual function prior to ADT had return of sexual function and libido on BAT.

After return to castrate T levels post-BAT, ten of 10 (100%) patients receiving second line therapy with either abiraterone (n=4/4) or an anti-androgen [enzalutamide (n=4/4), bicalutamide (n=1/1), nilutamide (n=1/1)] had a PSA decline (range 30.8-99.5%) (FIG. 3-5). Four of 4 patients receiving abiraterone and 3/4 patients on enzalutamide had >50% PSA decline. Of note, two subjects were re-challenged with a first-generation anti-androgen (i.e., nilutamide, bicalutamide) and one with enzalutamide after having previously progressed on these agents. These subjects achieved a 44.3%, 30.9% and 53.2% PSA decline upon initiation of nilutamide, bicalutamide and enzalutamide, respectively. The patient re-challenged with enzalutamide also previously progressed on abiraterone prior to enrolling in this study.

The lessons learned from this pilot trial were that high dose testosterone could be administered safely to patients with metastatic CRPC without producing worsening signs or symptoms due to prostate cancer. While formal testing was not performed, most patients reported improved quality of life with increased energy, less fatigue, increased libido and resumption of erectile function in those patients with preserved function prior to castrating therapy. PSA decline/response and objective responses were observed in 50% patients who completed three cycles of therapy. While PSA declines were observed, many patients with supposedly CRPC had an initial spike in PSA following the first injection of testosterone. These results suggest that, although these patients demonstrated progressive disease while on chronic castrating therapies as a requirement for enrollment in the trial, the CRPC cells must have continued expression of a functional AR axis as evidenced by the increased expression of an androgen responsive gene, PSA, in response to androgen replacement. Finally, 100% of patients demonstrated PSA response to androgen ablative therapies post-BAT suggesting exposure to BAT has the potential to reverse resistance and re-sensitize CRPC cells to androgen ablative therapies, such as abiraterone and enzalutamide.

3.5 Results from the RE-Sensitizing with Supra T to Overcome Resistance (RESTORE) Study

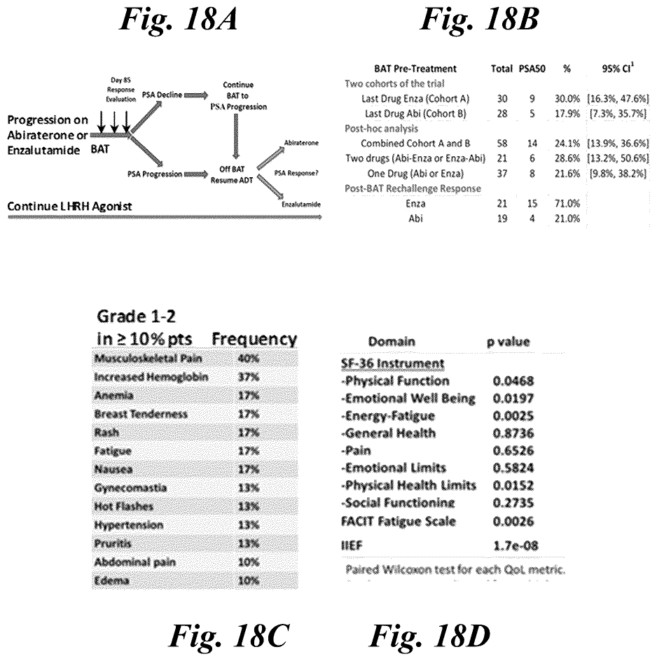

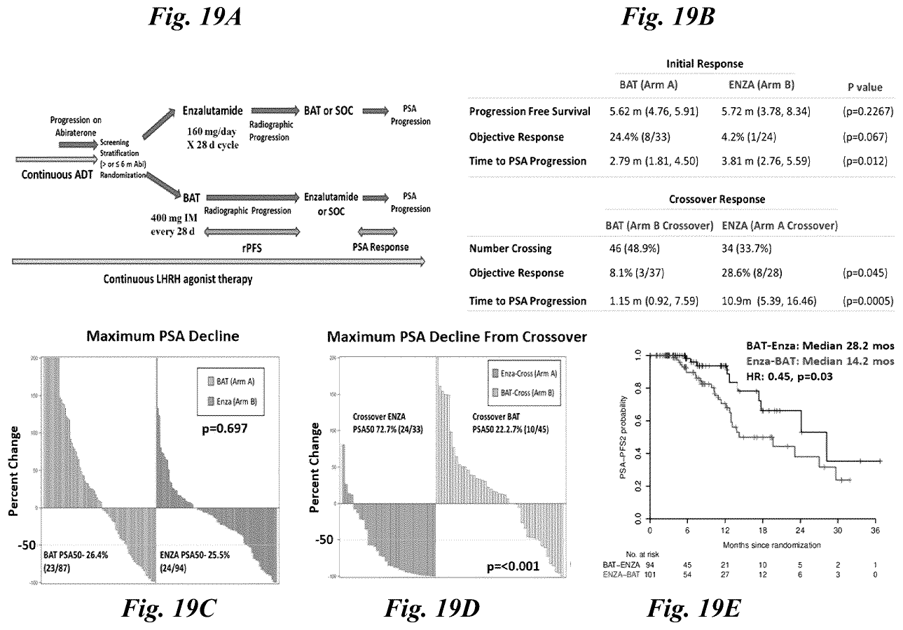

The RESTORE study (NCT02090114) was an NIH sponsored Phase II study designed to assess the safety and efficacy of BAT in patients (n=30 per cohort) with CRPC progressing on Enza (Cohort A) or Abi (Cohort B), FIG. 18A (34,39). The primary endpoint of the first part of the study was to assess PSA response to BAT. Secondary endpoints included objective response, safety, and quality of life. The second part of the study was to determine in BAT could re-sensitize patients to repeat exposure to second line therapy that patients were progressing on prior to receiving BAT. Thus, patients progressing on Enza were re-exposed to Enza after BAT and similarly for Abi. Both arms of this study met the primary endpoint. PSA₅₀ response to BAT was 30% in patient's post-Enza and 18% post-Abi, FIG. 18B. PSAso response was not significantly different in patients who had received two prior treatments (i.e., Enza-Abi or Abi-Enza) compared to only one. Overall duration of response to BAT and duration of response to re-exposure was longer in the post-Enza vs. post-Abi cohort. The PSAso response to re-exposure to Enza was 71% vs. only 21% for patients re-exposed to Abi, FIG. 19B (7). A third cohort was later added (Cohort C) for patients progressing on ADT alone. Overall, 90 patients were treated on this study over a 4-year period (2014-2018).

Adverse events (AEs) to BAT were primarily Grade 1-2 with most common being generalized musculoskeletal pain and sexual side effects (breast tenderness, hot flashes and gynecomastia), FIG. 18C. Serious AEs occurred in individual patients and were not attributed to BAT with the exception of grade 3 hypertension that occurred in 3 patients. QoL surveys showed significant improvement in the FACIT Fatigue Scale, IIEF survey, Physical Function, Emotional Well-Being and Energy-Fatigue on the SF-36 Instrument subscales, for BAT compared to baseline on ADT, FIG. 18D.

3.6 Results from the TRANSFORMER Study

The TRANSFORMER study (NCT02286921) was a DOD-sponsored randomized Phase II study designed to compare the efficacy of BAT vs. Enza in asymptomatic patients with CRPC progressing on Abi, FIG. 19A (2). The study was conducted at 17 academic centers across the US. The study was coordinated by the Prostate Cancer Disease Group clinical research team at Johns Hopkins, who performed regulatory oversight, study/site monitoring, and data analyses. From 2015-2018, 195 patients underwent 1:1 randomization to either standard dose Enza (n=101) or BAT (n=94) at 400 mg IM every 28 days. The primary endpoint was clinical/radiographic PFS. At time of progression, patients were given the option to crossover to the alternate therapy. The trial was designed to show a 50% improvement in the primary endpoint for BAT vs. Enza. The trial did not meet the primary endpoint with PFS of 5.62 m for the BAT arm and 5.72 m for the Enza arm (p=0.2267), FIG. 19B. Objective response was 24.4% for BAT and 4.2% for Enza (p=0.067). Best PSA₅₀ was 26.4% for BAT and 25.5% for Enza (p=0.697).

At time of progression, 46 (49%) of patients on the Enza arm crossed over to receive BAT and 34 (33.7%) crossed over from BAT to receive Enza and were available for analysis. Remarkably, the PSAso response for patients who received Enza after BAT was 72.7% compared to 25.5% for patients receiving Enza immediately after Abi, FIG. 19D. Objective response in the Enza post-BAT patients was 28.6% compared to 4.2% post-Abi, FIG. 19B. Time to PSA progression on Enza also was markedly improved, increasing almost 3-fold from 3.8 months immediately post-Abi to 10.9 months post-BAT, FIG. 19B. Finally, post-hoc analysis of PSA progression to the first and second stages of the study (PSA PFS2) for all patients, including censored patients who did not crossover, revealed a median of 14.2 months for the sequence of Enza crossing over to BAT, but 28.2 months for sequence of BAT crossing over to Enza, FIG. 19E. These results support our hypothesis that sequential therapy can overcome adaptive resistance and prolong therapeutic response. The logical extension of these findings and the hypothesis of this proposal is that patients are likely to respond again to further repeat cycles of BAT and Enza, but this treatment strategy has yet to be explored in the clinical trial setting.

3.7 Rationale for Combination Therapy with DFMO and High Dose Testosterone for CRPC

### 3.7.1 Supraphysiologic Androgen (SPA) Stimulates Ornithine Decarboxylase (ODC) Activity Increasing Polyamine Concentrations

Recently, we have studied how SPA affects the PCa cell and the cancer cell microenvironment with a goal of developing strategies to maximize growth-inhibitory effects and minimize adaptive effects. Global metabolomics of SKCaP-1R tumors after 14 days of SPA showed that the polyamines, putrescine and spermine, were the most enriched metabolites, while the polyamine precursors, ornithine and S-adenosylmethionine (SAM) were among the most depleted (FIG. 2A). Marked intracellular and extracellular accumulation of putrescine was confirmed by HPLC in LNCaP cells treated with the synthetic androgen R1881 at the supraphysiological dose of 10 nM (FIG. 2B). Moreover, SPA increased plasma putrescine abundance in patients with CRPC (FIG. 2C). This increase in putrescine abundance likely occurs due to enhanced expression of the first rate-limiting enzyme in polyamine synthesis, ornithine decarboxylase (ODC), as SPA increased ODC protein (FIG. 2D) and activity (FIG. 2E) in PCa cell lines. Beyond ODC, it is likely that multiple enzymes that promote polyamine synthesis and excretion are stimulated by AR activation, as spermidine/spermine N¹-acetyltransferase (SSAT) activity also was significantly increased by SPA (FIG. 2D). Moreover, we observed that the genes coding for S-adenosylmethionine decarboxylase (AMD1), spermine synthase (SMS), ornithine decarboxylase (ODC1), spermine oxidase (SMOX), acetylpolyamine oxidase (PAOX), spermidine synthase (SRM), and spermidine/spermine N¹-acetyltransferase (SAT1) were significantly downregulated in advanced PCa tumors after 22 weeks of ADT in the Rajan et. al dataset (40) and ODC1 expression positively correlated with AR activity in metastatic PCa tumors in the SU2C/PCF dataset (41). Together these data indicate that AR activation by SPA activates ODC to increase intracellular and excreted polyamines.

### 3.7.2 Inhibition of ODC Enhances Growth-Inhibitory Effects of SPA.

Polyamines are small, polycationic alkylamines that have a wide range of functions including stabilization of chromatin, maintenance of nucleic acid and protein synthesis, production of hypusine that modifies the translation elongation factor eIF5A, stimulation of MYC, regulation of pH, and modulation of nociception and immune responses (42). To assess the functional significance of stimulated polyamine synthesis by SPA, we tested the highly specific and clinically-utilized inhibitor of ODC, difluoromethylornithine (DFMO) (43). To assess a possible additive antitumor effect through combination with DFMO, we treated LNCaP and VCaP PCa cells with a low dose of R1881 (0.5 nM inhibits clonal survival by approximately 15%). DFMO inhibited clonal survival to a greater extent in R1881-treated cells than vehicle control-treated cells (FIG. 3A-B). Growth inhibition could be rescued by supplementing with exogenous putrescine (FIG. 3A-B), indicating that reduction of putrescine synthesis by DFMO increases growth suppression of CRPC by SPA. To assess whether combined therapy with SPA and DFMO has efficacy in vivo, we tested these agents in the subcutaneous castration-resistant syngeneic (immune-competent) mouse model, MYC-CaP-CR (44). This experiment indicated that combination treatment with SPA and DFMO was superior to either agent alone in reducing the rate of tumor growth in vivo (FIG. 3C). Altogether, these data suggest that SPA-stimulated synthesis of polyamines is adaptively protective for CRPC, and inhibiting this pathway using DFMO has potential to improve the efficacy of BAT.

### 3.7.3 History of Clinical Use of DFMO

The first phase I trial of DFMO was performed at our institution (Johns Hopkins) in 1984 for patients with advanced solid tumors or lymphomas (45), and shortly thereafter a phase I (5 patient) trial of DFMO and MGBG was performed for patients with advanced PCa in 1986 (46). It should be noted that DFMO was one of the first drugs to be approved for the treatment of Trypanosoma gambiense (African sleeping sickness) and was found to be extremely well-tolerated, even at high doses. Most recent clinical drug development of DFMO in cancer, however, has focused on assessing its potential as a chemo-preventive agent for cancer, owing to data that indicated elevated polyamines are permissive for cancer development (47). These trials suggest DFMO may reduce the incidence of non-melanoma skin cancers, but trials of breast and cervix cancer were negative (48-50). Large chemoprevention trials of DFMO for PCa have not been performed; however, initial studies suggest that DFMO can reduce prostate polyamine levels and the rate of growth of the prostate over 1 year (51,52). This extensive clinical experience using DFMO provides a wealth of dosing and safety data on this drug, and indicate that it is effective at inhibiting ODC and is generally well tolerated. While high doses of DFMO are associated with reversible ototoxicity and myelosuppression, doses less than 1 g/m²/d are not consistently associated with these side-effects (53). Importantly, most recent clinical evaluation of DFMO as a cancer therapy has been limited to CNS tumors (54,55). DFMO, however, has never been assessed for efficacy as treatment for PCa. Our preclinical data presented here provide strong rationale to repurpose this drug as a combination therapy with BAT for the treatment of CRPC.

3.7.4 DFMO has Potential to Improve Pain and Quality of Life for Patients with Advanced PCa

Prior studies have suggested that elevated polyamines occurring in the inflammatory microenvironment mediate nociception in part through activating the capsaicin receptor TRPV1 (56-59). Testosterone administration is described to induce pain flares in patients with PCa (60), and we have occasionally seen this occur in our clinical studies. For this reason, all clinical trials of BAT have been limited to asymptomatic patients without pain requiring opiates. Without wishing to be bound to any one particular theory, it is thought that pain flares induced by BAT in a small subset of patient, may be due in part to enhanced polyamine production in the tumor microenvironment. Therefore, DFMO has the potential to prevent pain for patients on this trial by reducing polyamine abundance in metastatic tumors.

### 3.8 Study Objectives

### 3.8.1 Primary Objectives

The primary objective of the study is to determine if treatment with the combination of DFMO plus BAT improves PSA response in asymptomatic patients with evidence of progressive metastatic CRPC post-treatment with abiraterone compared to historical controls.

### 3.8.2 Secondary Objectives

(1) Progression-free survival (PFS): time to radiographic or clinical progression or death [PCWG3 definition]; (2) PSA response rate (>50% PSA decline from baseline) at any point on trial; (3) incidence and severity of adverse events and serious adverse events graded according to CTCAE v5.0; (4) PSA progression-free survival (PSA-PFS) [PCWG3 criteria]; (5) objective response rate in those with measurable disease; (6) change in pain score on the short-form McGill Pain Questionnaire (SF-MPQ); and (7) overall survival.

### 3.8.3 Radiographic Progression

Patients will remain on treatment until evidence of radiographic or clinical progression. Radiographic progression-free survival is based on parameters suggested by PCWG3 and modified RECIST as follows: (1) on CT scan, radiographic progression will be defined by RECIST 1.1 criteria (i.e. >20% increase in the sum of target lesions); (2) on bone scan, radiographic progression will be defined by PCWG3 criteria as ≥2 new bone lesions; and (3) death from any cause also will be considered as progression.

### 3.8.4 Clinical Progression

Patients who do not meet the criteria of radiographic progression and are removed from study for worsening symptoms that are attributable to prostate cancer progression, will be considered to have clinical progression.

Time to clinical progression will be defined as the time from randomization to documentation in the CRF of any of the following (whichever occurs earlier): (1) cancer pain requiring initiation of chronic administration of opiate analgesia (oral opiate use for ≥3 weeks; parenteral opiate use for >7 days). Patients with cancer pain requiring opiate analgesia for relief also should be assessed by the investigator for the need for initiating systemic chemotherapy or palliative radiation; (2) development of a skeletal-related event (SRE): pathologic fracture, spinal cord compression, or need for surgical intervention or radiation therapy to the bone; (3) development of clinically significant symptoms due to loco-regional tumor progression (e.g., urinary obstruction) requiring surgical intervention or radiation therapy. Patients with bone pain due to pain flare after first two injection of testosterone can remain on study and are not considered to have clinical progression. If bone pain persists after first cycle of enzalutamide, patients will stop treatment and come off study. If pain resolves, but returns with next or subsequent cycles of testosterone, patients will stop treatment and come off study.

### 3.9 Treatment Plan

### 3.9.1 Study Design

Asymptomatic patients with mCRPC without pain due to prostate cancer progressing after treatment with Abi will be treated on an open label study to evaluate effectiveness of sequential treatment with the combination of DFMO and high dose testosterone in sequence with enzalutamide to improve primary and secondary outcomes.

### 3.9.2 Study Treatments and Treatment Scheme

### 3.9.2.1 Study Treatments

Patients will continue on ADT with LHRH agonist (i.e., Zoladex, Trelstar, Eligard, or Lupron) or LHRH antagonist (Degarelix or Relugolix) if not surgically castrated throughout the duration of the study to inhibit endogenous testosterone production. One cycle of treatment will be 119 days and will involve:
1. 7 days of DFMO at a dose of 1000 mg PO BID (D1-D7), followed by
2. 56 days of combined testosterone and DFMO (testosterone cypionate 400 mg IM on D8 and D36 with continued DFMO 1000 mg PO BID) (D8-D63), followed by
3. 56 days of enzalutamide (enzalutamide 160 mg PO daily) (D64-D119).
   3.9.2.2 Treatment Scheme

### 3.9.2.3. Dosing Delays and Modifications

Treatment will be given on indicated timepoints±7 days. Patients who develop seizures, pulmonary embolus or other thromboembolic event will be removed from study. There will be no dose modifications for testosterone in this study. Dose modification for enzalutamide will be according to the institution's standard of care practice. Enzalutamide dose reduction to label recommended dose of 80 mg/day is allowed for patients on concomitant CYP2C8 inhibitors (gemfibrozil) or strong CYP3A4 inducers. Dose reduction is at the discretion of the treating physician. Dose reduction for DFMO to 500 mg BID is allowed for patients who experience reversible ototoxicity. Patients may temporarily suspend study treatment if they experience toxicity that is considered to be related to study drugs and requires a dose to be held. Treatment delay will be allowed up to 4 weeks due to drug related toxicities. If, in the judgment of the investigator, the patient is likely to derive clinical benefit from resuming the study drugs after 4 weeks, the study drug may be restarted with the approval of the Medical Monitor.

Dose interruptions for reason(s) other than toxicity, such as surgical procedures, may be allowed with Medical Monitor approval. The acceptable length of interruption will depend on agreement between the investigator and the Medical Monitor.

If a dose of Enzalutamide of DFMO is missed for more than 6 hours, it will be skipped. If a dose is vomited do not replace the dose. Start with the next scheduled dose.

3.9.2.4 Removal of Patients from Study

A patient may be removed from the study for a variety of reasons, including: (1) as defined by the protocol, evidence of disease progression based on radiographic progression or worsening symptoms; (2) unacceptable adverse event(s), including: (a) patients develop new or worsening pain deemed by the investigator to be due to disease progression; (b) patients develop urinary outlet obstruction well documented and thought to be due to prostate cancer within the prostate and requiring urinary catheterization; (c) patients who develop grade 3 or higher liver function abnormalities with increase in bilirubin, AST (SGOT) or ALT (SGPT)≥2.5 times institutional upper limit of normal (ULN); (d) patients develop decreased renal function with serum creatinine≥2.5 times baseline level due to prostate cancer progression or drug toxicity; (e) patents develop hypersensitivity or anaphylactoid reactions to study drugs; (f) any evidence of severe dose limiting toxicities secondary to treatment with enzalutamide that cannot be controlled with standard therapy (e.g., nausea/vomiting not controlled with oral antiemetic regimen); (g) intercurrent illness that prevents further participation; (h) experiencing a treatment delay of longer than 4 weeks due to drug toxicity; however, if the patient is receiving clinical benefit, treatment may be delayed for longer than 4 weeks and then resumed at the discretion of the Investigator and the approval of the Medical Monitor; (i) patient refuses further treatment through the study and/or withdraws consent to participate; (j) patient is noncompliant with respect to taking drugs, keeping appointments, or having tests required for the evaluation of drug safety and efficacy; (k) general or specific changes in the patient's condition that render the patient unacceptable for further treatment in this study in the judgment of the investigator; (k) under no circumstance will care of a withdrawn patient be adversely affected by a decision to withdraw or be withdrawn from the study; and (l) deterioration in ECOG performance status to grade 3 or higher.

### 3.9.2.5 Criteria for Discontinuation of Study Treatment Due to Development of Clinical Progression Due to Prostate Cancer

Due to the nature of the treatment, there is concern that patients could experience potential development of prostate cancer related pain due to BAT. Significant worsening of pain was only observed in a small number of patients in the prior three studies that previously tested the use of testosterone as therapy for patients with asymptomatic CRPC. In addition, patients with baseline pain due to prostate cancer are excluded from enrolling in the study.

Patients with PSA progression, but with disease response or stable disease on imaging studies, will remain on study until clinical or radiographic progression criteria are met. Patients with radiographic disease progression will stop treatment and come off study. Patients with clinical progression due to pain flare after first two injections of testosterone can remain on study. If pain persists after first cycle of enzalutamide, patients will stop treatment and come off study. If pain resolves, but returns with next or subsequent cycles of testosterone, patients will stop treatment and come off study.

For this study, unequivocal clinical progression will be characterized as: (1) cancer pain requiring initiation of chronic administration of opiate analgesia (oral opiate use for ≥3 weeks; parenteral opiate use for ≥7 days. Patients with cancer pain requiring opiate analgesia for relief also should be assessed by the investigator for the need for initiating systemic chemotherapy or palliative radiation; (2) development of a skeletal-related event (SRE): pathologic fracture, spinal cord compression, or need for surgical intervention or radiation therapy to the bone; (3) development of clinically significant symptoms due to loco-regional tumor progression (e.g. urinary obstruction) requiring surgical intervention or radiation therapy. All patients meeting the criteria for unequivocal clinical progression should have repeat imaging studies (i.e., bone and CT scans) if not done within the past month of meeting said criteria.

### 3.9.2.6 Pain Management

Patients who develop new pain thought by the investigator to be due to prostate cancer should be treated according to NCCN guidelines for Adult Cancer Pain (Ver. 1.2014) in a stepwise fashion using the WHO Analgesic Ladder approach. Patients with pain that is managed without oral opioids after 3 weeks can continue on to the next cycle of the study. Patients with a continued requirement for oral opioids after 3 weeks will be removed from study. Patients who have recurrent pain requiring oral opioids on subsequent cycles also will be removed from study.

Patients with pain that occurs after initial testosterone injections may be experiencing testosterone-induced pain flare. These patients can remain on study with pain symptoms managed per NCCN guidelines. If pain persists after first cycle of enzalutamide, patients will stop treatment and come off study. If pain resolves, but returns with next or subsequent cycles of testosterone, patients will stop treatment and come off study.

### 3.9.2.7 Concomitant Therapy

The use of any concurrent medication from screening and while on study, prescription or over-the-counter, is to be recorded on the patient's CRF along with the reason the medication was taken. In addition, history of tobacco and alcohol use will be collected. Concurrent use of another clinical investigational drug or device while on study is prohibited. Supportive care medications are permitted with their use following institutional guidelines. For patients who did not undergo orchiectomy, concurrent treatment with LHRH analogue is mandatory and must be recorded. The following supportive care medications are considered permissible during the study: Conventional multivitamins, selenium and soy supplements; Systemic glucocorticoid administration such as “stress dose” glucocorticoid up to maximum of 4 mg/day dexamethasone or dexamethasone equivalent is permitted if clinically indicated. Selection of corticosteroid, dose and duration is at the discretion of the treating physician; Dutasteride or finasteride if being used to treat BPH and only if patients are on the medication for at least 3 months prior to Study Day 1; Bisphosphonate and denosumab usage is allowed only if patients are on the medication for at least 3 months prior to Study Day 1; Transfusions and hematopoietic growth factors per institutional practice guidelines.

### 3.9.2.8 Prohibited Concomitant Medications

Concomitant therapy during the treatment phase of the study with any of the following listed is prohibited: chemotherapy; immunotherapy; bicalutamide, nilutamide, flutamide, apalutamide, or darolutamide; systemic ketoconazole (or other azole drugs, such as fluconazole and itraconazole); diethylstilbestrol, PC-SPES, and other preparations, such as saw palmetto thought to have endocrine effects on prostate cancer; Radiopharmaceuticals, such as xofigo (²²³Ra), strontium (⁸⁹Sr) or samarium (¹⁵³Sm), and other experimental drugs or treatments.

### 3.10 Treatment Period

All required treatment and post-treatment study procedures and assessments must be done within 7 days (+/−) of the specified study visit date. Patients will be treated according to the following plan: (1) patients will continue on ADT with LHRH agonist (i.e., Zoladex, Trelstar, Eligard, or Lupron) or LHRH antagonist (Degarelix or Relugolix) if not surgically castrated throughout the duration of the study to inhibit endogenous testosterone production; (2) one cycle of treatment will be 119 days and will involve: (a) 7 days of DFMO at a dose of 1000 mg PO BID (Day 1-Day 7), followed by; (b) 56 days of combined testosterone and DFMO (testosterone cypionate 400 mg IM on Day 8 and Day 36 with continued DFMO 1000 mg PO BID) (Day 8-Day 63), followed by; (c) 56 days of enzalutamide (enzalutamide 160 mg PO daily) (Day 64-Day 119).

Other treatment protocols include: (1) patients will have a clinic visit on Day 8, Day 36, Day 64 of cycle 1 and Day 1, Day 36, Day 64 of subsequent cycles; (2) CBC and CMP will be measured on Day 1 and Day 64; (3) PSA will be measured on Day 8, Day 36, Day 64, Day 92 of cycle 1 and Day 1, Day 36, Day 64, and Day 92 of subsequent cycles. PSA may be measured by an outside laboratory if the same laboratory is used for every measurement; (4) CT and bone scan will be performed on Day 64 of cycle 1 and on Day 1 of subsequent cycles. Therefore scans will be performed every 8 weeks for the first 2 scans then every 16 weeks thereafter; (5) a soft tissue biopsy for applicable patients will be performed on C1D64; (6) blood for PBMCs and plasma will be collected on Cycle 1/Day 8 and Cycle 1/Day 64. Patients must be fasting for 3 hours prior to these blood draws; (7) blood for ctDNA will be collected on Cycle 1/Day 8, Cycle 1/Day 64, then Day 1 of subsequent cycles; (8) a pain survey will be performed on Day 1 and Day 64 of all cycles; and (9) audiogram will be performed on Day 1 of all cycles.

### 3.11 Tumor Biopsies

Patients with soft tissue disease amenable to biopsy as assessed by Interventional Radiology must agree to sequential biopsy to be eligible for study. Patients will have biopsy at screening and then repeated biopsy on CID64±2 days.

### 3.12 End of Study Visit (at the Time of Progression)

If patient withdraws from the study at any time or if patient has radiological or clinical progression, patient will come in for an end of study visit within 30 days after the last dose of study drug to have the following procedures done: (1) physical examination; (2) vital signs; (3) ECOG; (4) adverse events; (5) comprehensive metabolic panel, CBC, and serum PSA; (6) blood for CtDNA; (7) pain survey; and (8) all patients meeting the criteria for unequivocal clinical progression should have repeat imaging studies (i.e., bone and CT scans) if not done within the past month of meeting outlined/stated or required criteria. For patients with clinical progression that requires patient to come off study (i.e., worsening pain, obstructive symptoms) should be followed until resolution of the adverse event/toxicity or at least one month, whichever is later.

### 3.13 Overall Survival Assessment

All patients will be followed for survival every 6 months for three years from the off-treatment date. Information on patient's survival status and subsequent treatments will be collected and may be obtained by phone call, email, clinic visit, or medical records (e.g., physician notes/laboratory results of clinic or hospital visits.

### 3.14 Early Discontinuation

For patients, who come off the study treatments due to an adverse event or unacceptable toxicity related to the study drugs will come back to the clinic for an end of study visit. If early discontinuation is due to an adverse event or unacceptable toxicity related to any of the study treatments the patient should be followed until resolution of the adverse event/toxicity or at least one month, whichever is later.

### 3.15 Study Assessments

### 3.15.1 Assessing Response in Measurable Disease

In patients with measurable disease, tumor response will be evaluated using CT and bone scan. Patients will undergo CT scan and bone scan at screening, on C1D64, and on D1 of subsequent cycles (i.e., every 119 days). Progression for soft tissue lesions will be based on RECIST 1.1 criteria and for bone lesions based on PCWG3 criteria.

### 3.15.2 Pain Surveys

Pain response will be assessed using short-form McGill Pain Questionnaire (SF-MPQ) Survey.

### 3.15.3 Audiograms

If a significant threshold shift ≥15 dB at 2 or more frequencies occurs, treatment with DFMO will be held. A repeat audiogram will be performed after 4 weeks. If toxicity is resolved, the patient will remain on trial with 50% dose reduction of DEMO (500 mg BID). If the toxicity is persistent, the patient will be taken off trial.

### 3.16 Correlative Studies

### 3.16.1 Tumor Biopsies

Biopsy of soft tissue will be performed by Interventional Radiology at each site. Interventional Radiology will review baseline CT scan to determine if biopsiable metastasis present. For each soft tissue metastatic biopsy, an 18-gauge core biopsy needle will be used to obtain ≥1 cores for FFPE (first priority), ≥1 fresh cores for immediate processing for single cell RNAseq (second priority), and ≥1 fresh cores for flash-freezing in liquid nitrogen (third priority).

### 3.16.2 Peripheral Blood Mononuclear Cells (PBMCs)

PBMCs will be collected at indicated time points for phenotypic and functional characterization.

### 3.16.3 Plasma

Plasma will be collected for measurement of polyamine and other metabolite abundance.

3.16.4 Circulating tumor DNA (ctDNA)

Plasma will be collected for measurement of ctDNA at indicated time points.

### 3.17 Safety Assessment

Safety will be evaluated based on the incidence, severity, duration, causality, seriousness, and type of adverse events (AEs), and changes in the patient's physical examination, vital signs, and clinical laboratory results. Investigators will use the NCI CTC version 5.0 published 27 Nov. 2017 to assess the severity of AEs and toxicities. All observed or volunteered adverse events regardless of treatment group or causal relationship to study drug will be recorded on the adverse event page(s) of the case report form (CRF).

### 3.18 Study Calendar

| | | | Cycle 1 (119 days) | | | | | Cycles 2 and onward (each 119 days) | | | | | End of | End of |
|---|---|---|---|---|---|---|---|---|---|---|---|---|---|---|
| | | [symbol] | C1D1 | C1D8 | C1D35 | C1D84 | C1D92 | D1 | D8 | D35 | D84 | D92 | treatment [symbol] | study [symbol] |
| [symbol] | [symbol] | | C1D1-63 | | | | | D1-63 | | | | | | |
| | [symbol] | | | C1D8 | C1D35 | | | | D [symbol] | D33 | | | | |
| | [symbol] | | | | | C1D84- [symbol] | | | | | D [symbol] | | | |
| | [symbol] | [symbol] | | | | | | | | | | | | |
| [symbol] | History and Physical | X | | | | | | | | | | | | |
| | Clinic [symbol] | X | | X | X | X | | X | | X | X | | X | |
| | E [symbol] | X | | X | X | X | | X | | X | X | | X | |
| | Height | X | | | | | | | | | | | | |
| | Weight | X | | | | X | | X | | | X | | X | |
| | C [symbol] C | X | | | | X | | X | | | X | | X | |
| | CMP | X | | | | X | | X | | | X | | X | |
| | PSA | X | | X | X | X | X | X | | X | X | X | X | |
| | Serum Testosterone | X | | | | X | | | | | X | | | |
| | E [symbol] | X | | | | | | | | | | | | |
| | PTT [symbol] NR | X | | | | | | | | | | | | |
| | Tumor biopsy [symbol] | X | | | | X | | | | | | | | |
| | Bone sca [symbol] | X | | | | X | | X | | | | | | |
| | CT [symbol] | X | | | | X | | X | | | | | | |
| | Blood Sample for [symbol] | X | | X | | X | | | | | | | | |
| | Blood Sample for [symbol] | X | | X | | X | | | | | | | | |
| | Blood Sample for [symbol] | X | | X | | X | | X | | | | | X | |
| | Pain [symbol] | X | | | | X | | X | | | | | X | |
| | Adverse Events | X | | X | X | X | | X | | X | X | | X | |
| | Au [symbol] | X | | | | | | X | | | | | | |
| | Survival Assesment [symbol] | | | | | | | | | | | | | [symbol] |

C = Cycle
D =

[symbol]

[symbol]

[symbol]

indicates data missing or illegible when filed

### 3.19 Statistical Methods

This is an open label, single arm study to evaluate the effect of combination of DFMO and High Dose Testosterone and in sequence with enzalutamide in patients with metastatic castrate-resistant prostate cancer (CRPC) post-treatment with abiraterone.

### 3.19.1 Sample Size Determination

The primary endpoint is the PSA₅₀ response rate after 64 days of DFMO+BAT. In prior studies the PSA₅₀ response to 64 days of BAT monotherapy is approximately 25% in patients with mCRPC progressing on abiraterone (1-4). We expect that the addition of DFMO to BAT will increase the PSA₅₀ response rate on C1D64 to 40%. The sample size is calculated to detect an improved PSAso response rate from 25% to 40% by C1D64. A two-stage design is planned. A total of 24 patients will be entered in the first stage. If <=4 subjects have PSAso response, the treatment will be terminated and we will conclude the regimen is ineffective. If >=5 subjects respond, then additional 22 patients will be studied. If a total of 15 or fewer subjects have PSA₅₀ response by C1D64 in stage one and two combined, we consider the regimen ineffective. If a total of 16 or more respond, we conclude the regimen is promising and warrant further study. The maximum sample size will be 46 evaluable patients. This design provides 81% power to detect a difference of 15% of PSA₅₀ response rate with a type I error of 0.09 (target type I error of 0.1). Accounting for an approximate 10% drop-out rate before evaluation, the trial will enroll 50 patients. The calculation is performed using PASS 2022 software.

### 3.19.2 Analysis of Primary Endpoint

The PSA₅₀ response rate, defined as the proportion of evaluable patients who have ≥50% PSA decline from baseline, will be reported along with 95% confidence intervals. A patient will be considered evaluable if he receives at least one dose of DFMO in combination with BAT.

### 3.19.3 Analysis of Secondary Efficacy Endpoints

PFS is defined as the time from the date of first dose of study treatment to the date of first documented radiological progression per RECIST 1.1 for soft tissue or PCWG3 for bone lesions, or clinical progression or death, whichever occurs first. If a patient has not had the event at the date of data cut-off for analysis, PFS will be censored at the time of the last tumor assessment before the cut-off date. Time to PSA progression (PSA-PFS) is the time from the date of first dose to the time of PSA progression. PSA progression will be defined per PCWG3 criteria. When there is decline from baseline, the PSA progression date is defined as the first date that a ≥25% increase and an absolute increase of ≥2 ng/ml above the nadir is documented, and which is confirmed by a second value obtained 3 or more weeks later. For those with no PSA decline from baseline, PSA progression date is the first date of PSA increase that is ≥25% and an absolute increase of ≥2 ng/mL above the baseline after at least 12 weeks of treatment. Baseline PSA is the PSA measure at screening visit. Patients without a PSA progression will be censored at the last PSA assessment date. Overall survival (OS) is the time from the first dose to the date of death due to any cause. Time to event endpoints of PFS, PSA-PFS and OS will be summarized using Kaplan-Meier method. Overall response will be estimated as the proportion of subjects with measurable disease who achieve cither complete response or partial response per RECIST 1.1. Maximum percent decline of PSA from baseline for individual patients will be presented in waterfall plots. PSA₅₀ response rate at any time of the treatment will be estimated.

### 3.19.4 Safety Analysis

Incidence of adverse events will be summarized by system organ class and preferred terms within a system organ class based on NCI CTCAE version 5.0. We will monitor the toxicity throughout the trial. Safety monitoring and stopping rule: We will monitor the toxicity throughout the trial. Any adverse event of grade 3 or higher will be an endpoint for the purpose of safety stopping rule. A rate of such events that exceeds 30% will be considered unacceptable. A Bayesian monitoring rule will be used to halt patient enrollment if, at any time, the posterior distribution of risk being greater than 0.30 is 60% or higher. The monitoring rule is based on a beta (1.5, 5.5) prior distribution. This means that our prior guess of the proportion of AEs is 21%, and there is 90% probability that this proportion is below 42%. The decision rule for toxicity stopping is as follows:

| | | | | | | | | | | | | | | | |
|---|---|---|---|---|---|---|---|---|---|---|---|---|---|---|---|
| Study suspension if | 2 | 3 | 4 | 5 | 6 | 7 | 8 | 9 | 10 | 11 | 12 | 13 | 14 | 15 | 16 |
| number of patients | | | | | | | | | | | | | | | |
| with grade 3 or 4 | | | | | | | | | | | | | | | |
| AEs>=: | | | | | | | | | | | | | | | |
| When total number of | 3 | 6 | 9 | 12 | 15 | 18 | 22 | 25 | 28 | 31 | 35 | 38 | 41 | 44 | 48 |
| patients enrolled is: | | | | | | | | | | | | | | | |

If a patient has recurrent AEs of the same type, we only count it as one event at the highest grade. If a patient has multiple different AEs, only one event of the highest grade is counted toward this stopping rule.

The operating characteristics of the stopping rule are shown below and are based on 10,000 simulations:

| | | | | | | | |
|---|---|---|---|---|---|---|---|
| Risk of AE | 0.10 | 0.15 | 0.20 | 0.25 | 0.30 | 0.35 | 0.40 |
| % of Time study | 4.1 | 10.8 | 23 | 41.3 | 64.7 | 83.6 | 94.4 |
| stops | | | | | | | |
| Expected sample | 48.1 | 45.2 | 40.4 | 33.9 | 25.7 | 18.5 | 12.8 |
| size | | | | | | | |

### 3.19.5 Analysis of Pain Scores

Pain score will be assessed using the short-form McGill Pain Questionnaire (SF-MPQ). Summary statistics of the scores will be reported at baseline and when acquired (on D1 and D64 of each cycle). Changes in pain scores pre- and post-treatment will be evaluated at each follow-up time by paired-sample t-tests or nonparametric Wilcoxon signed rank tests as appropriate. In addition, mixture effect models will be fitted for accessing the changes of pain score over time.

### 3.20 References Example 3

1. Teply, B. A. et al. Bipolar androgen therapy in patients with metastatic castration-resistant prostate cancer after progression on enzalutamide: an open-label, phase 2, multicohort study. Lancet Oncol. 19, 76-86 (2018).
2. Denmeade, S. R. et al. TRANSFORMER: A randomized phase II study comparing bipolar androgen therapy versus enzalutamide in asymptomatic patients with castration-resistant metastatic prostate cancer. J. Clin. Oncol. JCO2002759 (2021).
3. Sena, L. A. et al. Bipolar androgen therapy sensitizes castration-resistant prostate cancer to subsequent androgen receptor ablative therapy. Eur. J. Cancer 144, 302-309 (2020).
4. Markowski, M. C. et al. A multicohort open-label phase II trial of bipolar androgen therapy in patients with metastatic castration-resistant prostate cancer (RESTORE): A comparison of post-abiraterone versus post-enzalutamide cohorts. Eur. Urol. (2020) doi:10.1016/j.eururo.2020.06.042.
5. Jemal, A. et al. Cancer statistics, 2008. CA Cancer J. Clin. 58, 71-96 (2008).
6. Huggins, C. Studies on prostatic cancer. Arch. Surg. 43, 209 (1941).
7. Crawford, E. D. et al. A controlled trial of leuprolide with and without flutamide in prostatic carcinoma. N. Engl. J. Med. 321, 419-424 (1989).
8. Laufer, M., Denmeade, S. R., Sinibaldi, V. J., Carducci, M. A. & Eisenberger, M. A. Complete androgen blockade for prostate cancer: What went wrong? J. Urol. 3-9 (2000).
9. Linja, M. J. et al. Amplification and overexpression of androgen receptor gene in hormone-refractory prostate cancer. Cancer Res. 61, 3550-3555 (2001).
10. Shah, R. B. et al. Androgen-independent prostate cancer is a heterogeneous group of diseases. Cancer Res. 64, 9209-9216 (2004).
11. Chen, C. D. et al. Molecular determinants of resistance to antiandrogen therapy. Nat. Med. 10, 33-39 (2004).
12. Isaacs, J. T. et al. Adaptive auto-regulation of androgen receptor provides a paradigm shifting rationale for bipolar androgen therapy (BAT) for castrate resistant human prostate cancer. Prostate 72, 1491-1505 (2012).
13. de Bono, J. S. et al. Abiraterone and increased survival in metastatic prostate cancer. N. Engl. J. Med. 364, 1995-2005 (2011).
14. Scher, H. I. et al. Increased survival with enzalutamide in prostate cancer after chemotherapy. N. Engl. J. Med. 367, 1187-1197 (2012).
15. Ryan, C. J., Molina, A. & Griffin, T. Abiraterone in metastatic prostate cancer. The New England journal of medicine vol. 368 1458-1459 (2013).
16. Beer, T. M. et al. Enzalutamide in metastatic prostate cancer before chemotherapy. N. Engl. J. Med. 371, 424-433 (2014).
17. Umekita, Y., Hiipakka, R. A., Kokontis, J. M. & Liao, S. Human prostate tumor growth in athymic mice: inhibition by androgens and stimulation by finasteride. Proc. Natl. Acad. Sci. U.S.A 93, 11802-11807 (1996).
18. Litvinov, I. V. et al. Androgen receptor as a licensing factor for DNA replication in androgen-sensitive prostate cancer cells. Proc. Natl. Acad. Sci. U.S.A 103, 15085-15090 (2006).
19. Vander Griend, D. J., Litvinov, I. V. & Isaacs, J. T. Stabilizing androgen receptor in mitosis inhibits prostate cancer proliferation. Cell Cycle 6, 647-651 (2007).
20. Haffner, M. C. et al. Androgen-induced TOP2B-mediated double-strand breaks and prostate cancer gene rearrangements. Nat. Genet. 42, 668-675 (2010).
21. Dehm, S. M., Schmidt, L. J., Heemers, H. V., Vessella, R. L. & Tindall, D. J. Splicing of a novel androgen receptor exon generates a constitutively active androgen receptor that mediates prostate cancer therapy resistance. Cancer Res. 68, 5469-5477 (2008).
22. Hu, R. et al. Ligand-independent androgen receptor variants derived from splicing of cryptic exons signify hormone-refractory prostate cancer. Cancer Res. 69, 16-22 (2009).
23. Thelen, P., Heinrich, E., Bremmer, F., Trojan, L. & Strauss, A. Testosterone boosts for treatment of castration resistant prostate cancer: an experimental implementation of intermittent androgen deprivation. Prostate 73, 1699-1709 (2013).
24. Denmeade, S. R. & Isaacs, J. T. Bipolar androgen therapy: the rationale for rapid cycling of supraphysiologic androgen/ablation in patients with castration resistant prostate cancer. Prostate 70, 1600-1607 (2010).
25. Noonan, K., Ellard, S. & Chi, K. Clinical activity of abiraterone acetate (AA) after progression on Mdv3100 in patients with metastatic castration resistant prostate cancer (MCRPC). Ann. Oncol. 23, ix305 (2012).
26. Loriot, Y. et al. Antitumour activity of abiraterone acetate against metastatic castration-resistant prostate cancer progressing after docetaxel and enzalutamide (MDV3100). Ann. Oncol. 24, 1807-1812 (2013).
27. Schrader, A. J. et al. Enzalutamide in castration-resistant prostate cancer patients progressing after docetaxel and abiraterone. Eur. Urol. 65, 30-36 (2014).
28. Bianchini, D. et al. Abiraterone in patients with metastatic castration-resistant prostate cancer progressing after docetaxel and MDV3100: A multicentre study. Ann. Oncol. 23, ix304-ix305 (2012).
29. Zhang, T. et al. Clinical benefit of docetaxel or enzalutamide after progression on first-line abiraterone acetate and prednisone in patients with metastatic castration resistant prostate cancer (mCRPC). J. Clin. Oncol. 32, e16031-e16031 (2014).
30. Suzman, D. L., Luber, B., Schweizer, M. T., Nadal, R. & Antonarakis, E. S. Clinical activity of enzalutamide versus docetaxel in patients with castration-resistant prostate cancer progressing after abiraterone. Prostate 74, 1278-1285 (2014).
31. Prout, G. R., Jr & Brewer, W. R. Response of patients with advanced prostatic carcinoma to exogenous administration of testosterone. Cancer 20, 1871-1878 (1967).
32. Donati, R. M., Ellis, H. & Gallagher, N. I. Testosterone potentiated 32P therapy in prostatic carcinoma. Cancer 19, 1088-1090 (1966).
33. Manni, A. et al. Androgen priming and chemotherapy in advanced prostate cancer: evaluation of determinants of clinical outcome. J. Clin. Oncol. 6, 1456-1466 (1988).
34. Szmulewitz, R. et al. A randomized phase 1 study of testosterone replacement for patients with low-risk castration-resistant prostate cancer. Eur. Urol. 56, 97-103 (2009).
35. Morris, M. J. et al. Phase 1 trial of high-dose exogenous testosterone in patients with castration-resistant metastatic prostate cancer. Eur. Urol. 56, 237-244 (2009).
36. Behre, H. M. & Nieschlag, E. Testosterone preparations for clinical use in males. in Testosterone (eds. Nieschlag, E., Behre, H. M. & Nieschlag, S.) 309-335 (Cambridge University Press, 2012).
37. Schweizer, M. T. et al. Effect of bipolar androgen therapy for asymptomatic patients with castration-resistant prostate cancer: results from a pilot clinical study. Sci. Transl. Med. 7, 269ra2 (2015).
38. Hussain, M. H., Pienta, K. J., Redman, B. G., Cummings, G. D. & Flaherty, L. E. Oral etoposide in the treatment of hormone-refractory prostate cancer. Cancer 74, 100-103 (1994).
39. Teply, B. A. et al. Bipolar androgen therapy in patients with metastatic castration-resistant prostate cancer after progression on enzalutamide: an open-label, phase 2, multicohort study. Lancet Oncol. 19, 76-86 (2018).
40. Rajan, P. et al. Next-generation sequencing of advanced prostate cancer treated with androgen-deprivation therapy. Eur. Urol. 66, 32-39 (2014).
41. Abida, W. et al. Genomic correlates of clinical outcome in advanced prostate cancer. Proc. Natl. Acad. Sci. U.S.A 116, 11428-11436 (2019).
42. Casero, R. A., Jr, Murray Stewart, T. & Pegg, A. E. Polyamine metabolism and cancer: treatments, challenges and opportunities. Nat. Rev. Cancer 18, 681-695 (2018).
43. Prakash, N. J., Schechter, P. J., Grove, J. & Koch-Weser, J. Effect of alpha-difluoromethylornithine, an enzyme-activated irreversible inhibitor of ornithine decarboxylase, on L1210 leukemia in mice. Cancer Res. 38, 3059-3062 (1978).
44. Ellis, L., Lehet, K., Ramakrishnan, S., Adelaiye, R. & Pili, R. Development of a castrate resistant transplant tumor model of prostate cancer. Prostate 72, 587-591 (2012).
45. Abeloff, M. D. et al. Phase I trial and pharmacokinetic studies of alpha-difluoromethylornithine—an inhibitor of polyamine biosynthesis. J. Clin. Oncol. 2, 124-130 (1984).
46. Herr, H. W., Warrel, R. P. & Burchenal, J. H. Phase I trial of α-difluoromethyl ornithine (DFMO) and methylglyoxal bis(guanylhydrazone) (MGBG) in patients with advanced prostatic cancer. Urology 28, 508-511 (1986).
47. Shukla-Dave, A. et al. Ornithine decarboxylase is sufficient for prostate tumorigenesis via androgen receptor signaling. Am. J. Pathol. 186, 3131-3145 (2016).
48. Alberts, D. S. et al. Chemoprevention of human actinic keratoses by topical 2-(difluoromethyl)-dl-ornithine. Cancer Epidemiol. Biomarkers Prev. 9, 1281-1286 (2000).
49. Fabian, C. J. et al. A phase II breast cancer chemoprevention trial of oral alpha-difluoromethylornithine: breast tissue, imaging, and serum and urine biomarkers. Clin. Cancer Res. 8, 3105-3117 (2002).
50. Vlastos, A.-T. et al. Results of a phase II double-blinded randomized clinical trial of difluoromethylornithine for cervical intraepithelial neoplasia grades 2 to 3. Clin. Cancer Res. 11, 390-396 (2005).
51. Simoneau, A. R., Gerner, E. W., Phung, M., McLaren, C. E. & Meyskens, F. L.—difluoromethylornithine and polyamine levels in the human prostate: Results of a phase Ila trial. J. Natl. Cancer Inst. 93, 57-59 (2001).
52. Simoneau, A. R. et al. The effect of difluoromethylornithine on decreasing prostate size and polyamines in patients: results of a year-long phase IIb randomized placebo-controlled chemoprevention trial. Cancer Epidemiol. Biomarkers Prev. 17, 292-299 (2008).
53. Doyle, K. J., McLaren, C. E., Shanks, J. E., Galus, C. M. & Meyskens, F. L. Effects of difluoromethylornithine chemoprevention on audiometry thresholds and otoacoustic emissions. Arch. Otolaryngol. Head. Neck Surg. 127, 553-558 (2001).
54. Levin, V. A. et al. Phase III randomized study of postradiotherapy chemotherapy with alpha-difluoromethylornithine-procarbazine, N-(2-chloroethyl)-N′-cyclohexyl-N-nitrosurea, vincristine (DFMO-PCV) versus PCV for glioblastoma multiforme. Clin. Cancer Res. 6, 3878-3884 (2000).
55. Levin, V. A. et al. Phase III randomized study of postradiotherapy chemotherapy with combination alpha-difluoromethylornithine-PCV versus PCV for anaplastic gliomas. Clin. Cancer Res. 9, 981-990 (2003).
56. Tan-No, K. et al. Intrathecally administered spermine produces the scratching, biting and licking behaviour in mice. Pain 86, 55-61 (2000).
57. Ahern, G. P., Wang, X. & Miyares, R. L. Polyamines are potent ligands for the capsaicin receptor TRPV1. J. Biol. Chem. 281, 8991-8995 (2006).
58. Rivat, C. et al. Polyamine deficient diet to relieve pain hypersensitivity. Pain 137, 125-137 (2008).
59. Silva, M. A. et al. Role of peripheral polyamines in the development of inflammatory pain. Biochem. Pharmacol. 82, 269-277 (2011).
60. Tagnon, H. J., Schulman, P., Whitmore, W. F. & Leone, L. A. Prostatic fibrinolysin. Am. J. Med. 15, 875-884 (1953).

### Example 4

### Polyamine Synthesis is Driven by the Androgen Receptor and can be a Vulnerability for Prostate Cancer

### 4.1 Background

The androgen receptor (AR) is a nuclear hormone receptor with context-dependent function. While it drives cancer progression in untreated prostate cancer (PCa), we and others have shown that it can paradoxically suppress cancer progression in castration-resistant PCa treated with supraphysiological androgens (SPA) (1). This switch in function appears to occur primarily when ligand-independent AR activity markedly increases as a mechanism of resistance to therapeutic AR blockade (2,3). Clinical trials have demonstrated that periodic administration of SPA (testosterone cypionate 400 mg intramuscularly every 28 days, clinically referred to as bipolar androgen therapy, BAT) results in objective responses for approximately 30% of patients with metastatic castration-resistant PCa and portends a median progression-free survival of 6 months (4). BAT is associated with few negative side-effects and several positive side-effects, including improved quality of life, and is inexpensive (4). Therefore, BAT seems to be an ideal foundation on which to layer additional systemic therapies to enhance efficacy. To nominate which additional therapies may be beneficial, a detailed understanding of molecular consequences of SPA may be helpful.

The AR functions as a transcription factor to exert downstream effects. It regulates expression of hundreds-thousands of genes, the complement of which depends on abundance and activity of the AR and its interactors, such as FOXA1 and HOXB13 (5-7). We and others have previously reported that in PCa with high AR activity, SPA results in transcriptional suppression of the protooncogene MYC (3). Notably, many AR and MYC target genes are proteins that regulate metabolism, and modulation of AR and MYC can reprogram cellular metabolism (8-10). This is true even in non-malignant contexts—for example, in skeletal muscle, AR activation by androgens stimulates anabolic metabolism to increase muscle hypertrophy (11-13). Metabolism is tightly connected with signaling pathways including epigenetic regulation of gene expression and thus can have profound consequences on cellular fate and function (14). Therefore, without wishing to be bound to any one particular theory, it was thought that AR activation by SPA reprograms the cellular metabolism of PCa and these cancer-cell intrinsic metabolic changes can modify therapeutic efficacy.

This Example demonstrates that SPA can alter PCa metabolism in part by increasing de novo polyamine synthesis. This increase in polyamine synthesis is driven by AR binding upstream of the ODC1 gene, which encodes ornithine decarboxylase (ODC), one of the rate-limiting enzymes in this pathway, and is paradoxically augmented by downregulation of MYC, which antagonizes AR induction of ODC. SPA-stimulated polyamine synthesis facilitates resistance to growth inhibition by SPA, as pharmacologic disruption of this signaling axis enhances growth inhibition by SPA. This occurs in part through activation of AMD1 and depletion of S-adenosylmethionine (SAM) pools leading to enhanced repression of MYC.

### 4.2 Results

### 4.2.1 Androgens Increase De Novo Polyamine Synthesis and Secretion in Prostate Cancer Models

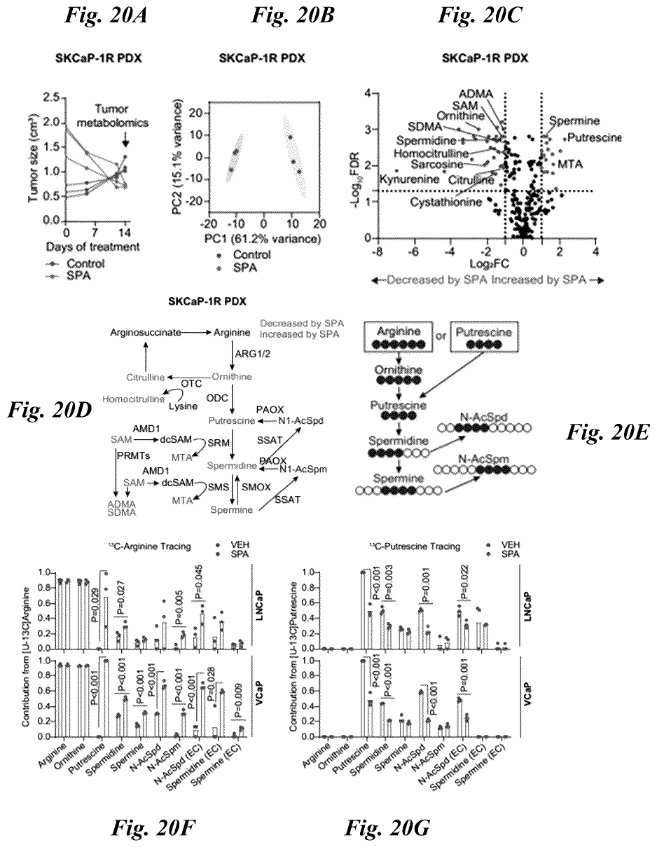

To globally assess how supraphysiological androgen (SPA) affects the metabolism of PCa, SKCaP-1R patient-derived xenograft tumors of similar size from untreated and SPA-treated mice were subject to global metabolomics by capillary electrophoresis mass spectrometry (FIG. 20A). SkCaP-1R is derived from a metastasis of a patient with metastatic castration-resistant PCa who had progressed through multiple systemic therapies including abiraterone, enzalutamide, docetaxel, and carboplatin, grows subcutaneously in castrated NSG mice, exhibits high expression of the androgen receptor (AR), and regresses following treatment with SPA (3, 16). As expected, the metabolome of tumors exposed to SPA was different from that of untreated tumors by principal component analysis (FIG. 20B). The most increased metabolite by SPA was the polyamine putrescine (FIG. 20C). Ornithine, the substrate of ornithine decarboxylase (ODC) to generate putrescine in the polyamine synthesis pathway (17), was significantly decreased by SPA, as were citrulline and homocitrulline, which are products of ornithine transcarbamylase (OTC) downstream of ornithine in the urea cycle (FIG. 20C-FIG. 20D). This suggested that SPA may deplete ornithine in favor of production of putrescine rather than citrulline. We also observed that despite increased abundance of putrescine, spermidine abundance was decreased while the products of spermine synthase (SMS), spermine and methylthioadenosine (MTA), were significantly increased (FIG. 20C-FIG. 20D). Moreover, S-adenosylmethionine (SAM), the substrate of S-adenosylmethionine decarboxylase 1 (AMD1) and precursor for decarboxylated SAM utilized by spermidine synthase (SRM) and SMS, was decreased by SPA, as were asymmetric dimethylarginine (ADMA) and symmetric dimethylarginine (SDMA), which are formed by methylation of arginine using SAM as a methyl donor (FIG. 20C-FIG. 20D). This suggested that SPA may deplete SAM in favor of production of MTA, spermidine, and spermine rather than methylated proteins.

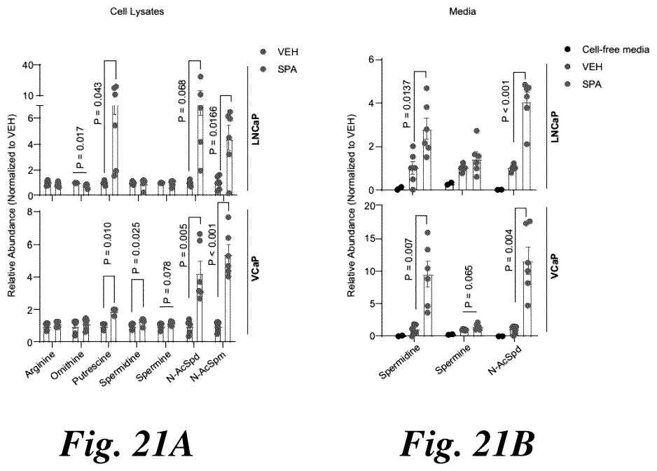

Without wishing to be bound to any one particular theory, it was thought that the mechanism by which SPA increased putrescine was through increased de novo synthesis given concurrent depletion of the upstream metabolite ornithine. In most mammalian cells, ornithine is produced from arginine through arginase (ARG1 and ARG2). Thus, we traced the fate of uniformly-labeled ¹³C-arginine or ¹³C-putrescine in the AR-positive PCa cell lines LNCaP and VCaP treated with SPA (FIG. 20H). While equal labeling of intracellular arginine and ornithine from ¹³C-arginine was observed in vehicle- and SPA-treated cells, SPA markedly enhanced labeled putrescine and downstream polyamines from ¹³C-arginine, indicating that SPA increased polyamine synthesis from arginine (FIG. 20I). In contrast, ¹³C-putrescine tracing revealed that SPA decreased the proportion of intracellular putrescine and downstream polyamines derived from extracellular putrescine (FIG. 20J). Interestingly, we observed that in these cell lines, SPA not only increased abundance of intracellular polyamines (FIG. 21A), but it also increased polyamines in the media of treated cells (FIG. 21B), which were also derived from de novo synthesis from arginine (FIG. 20F). Together, these data suggest that SPA increases de novo synthesis and secretion of polyamines from arginine in models of AR-positive PCa.

### 4.2.2 AR Regulates Expression of Polyamine Synthesis Enzymes

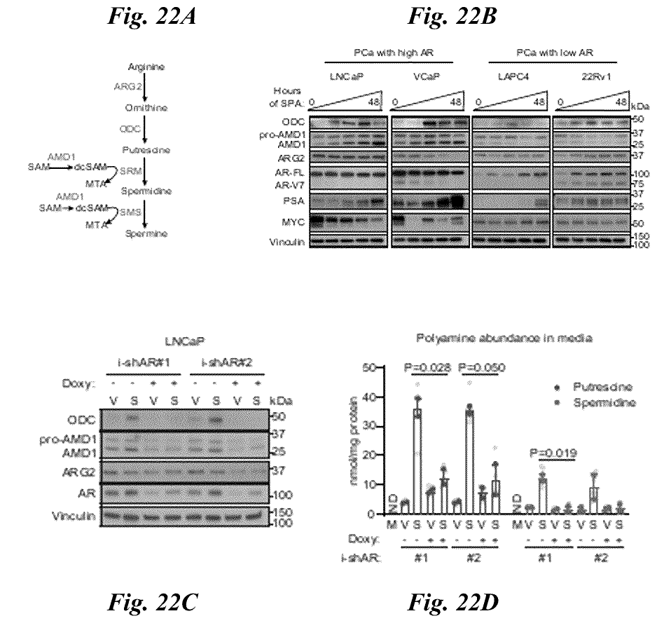

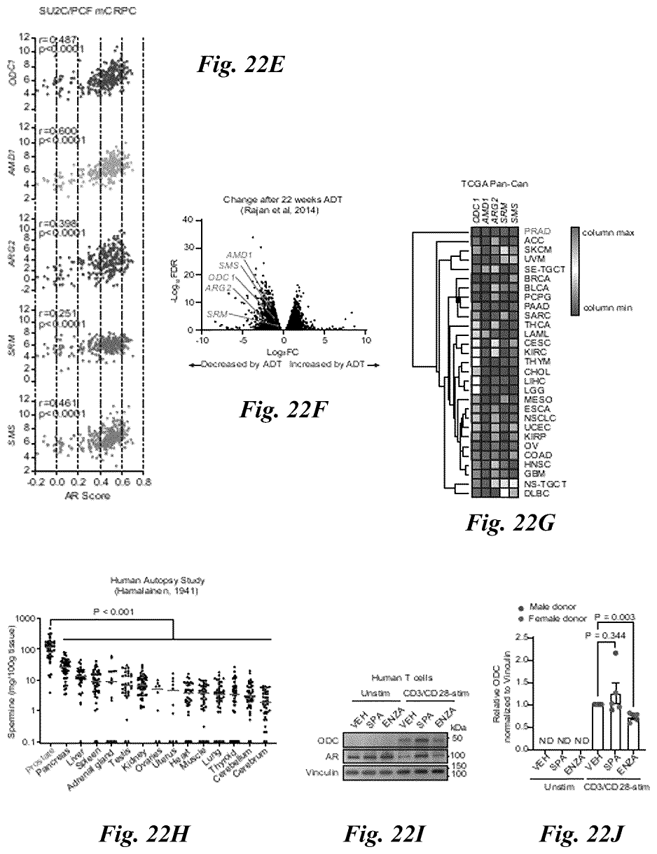

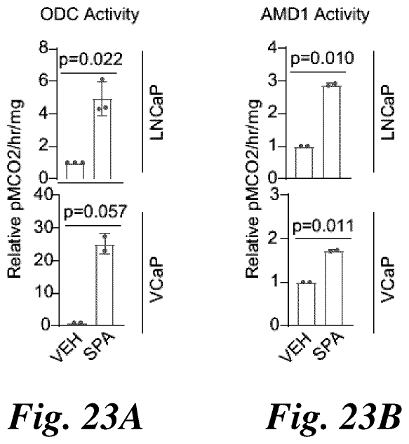

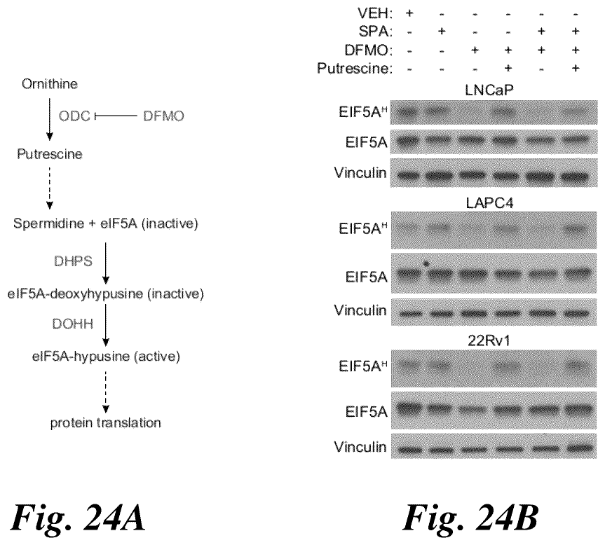

Over fifty years ago, Pegg and Williams-Ashman demonstrated that androgen deprivation by surgical castration resulted in decreased ODC and AMD1 activity and polyamine abundance in the rat ventral prostate within 6 hours, which was reversible by administration of testosterone to the animals (18,19). Subsequent studies in rodents indicated that androgens acutely can increase ODC1 mRNA, and prolonged administration of androgens can increase the half-life of ODC protein from 15 minutes to 100-150 minutes (20-22). In 1997, Betts et al. determined that the human prostate cancer cell line LNCaP increases ODC1 mRNA with similar kinetics to KLK3 mRNA (encoding PSA) in response to androgens (23). Therefore we hypothesized that SPA increases de novo synthesis of polyamines in PCa through AR regulation of enzymes mediating polyamine synthesis, including ODC, AMD1, ARG2, SRM, and SMS (FIG. 22A). Indeed, we observed that SPA increased the abundance and activity of ODC over time with similar kinetics as PSA across several AR-positive PCa cell lines (FIG. 22B and FIG. 23A). In contrast, SPA increased the abundance of AMD1 only in cell lines that highly express the AR (FIG. 22B and FIG. 23B). SPA-mediated increases in ODC and AMD1 and consequent polyamine secretion occurred through AR, as they were reduced by inducible knock-down of AR (FIG. 22C-FIG. 22D). Although ARG2 was not induced by SPA, it appeared to be regulated by AR, as inducible knock-down of AR in LNCaP cells reduced its expression (FIG. 22C), as did SPA in VCAP cells in association with the auto-downregulation of AR that occurs rapidly in these cells (FIG. 22B). Assessment of SRM and SMS protein abundance in PCa cell lines was not performed, as we lacked specific antibodies for these proteins. However, the abundance of all of these mRNA transcripts (ODC1, AMD1, ARG2, SRM, and SMS) positively correlated with an AR activity score in prostate cancer biopsies obtained from patients with metastatic castration-resistant PCa in the SU2C/PCF Dream Team dataset (n=264) (24) (FIG. 22D) and were decreased in PCa biopsies following androgen-deprivation therapy in the Rajan et al dataset (n=7) (25) (FIG. 22E). Looking across cancer types in the TCGA Pan-Cancer Atlas study, (26) PCa exhibited the highest expression of ODC, AMD1, ARG2, and SMS of any cancer type (FIG. 22F), which we suspect is due to its uniquely high expression and activity of AR. Notably, SRM was not especially abundant in PCa, had a relatively weak correlation with AR activity, and had the least change in expression by ADT (FIG. 22C-FIG. 22E), suggesting that expression of this enzyme is unlikely to be regulated by AR. This may explain why we observed that SPA led to accumulation of putrescine and spermine, but not spermidine in the SkCaP model (FIG. 20C). Spermidine enables activation of the translation factor eIF5A, as it is the precursor for hypusination of eIF5A (FIG. 24A). In accordance with minimal alteration of SRM and spermidine pools by SPA, we noted that SPA also did not alter abundance of hypusinated eIF5A (FIG. 24B). Altogether these data suggest that AR can regulate abundance of polyamine synthesis enzymes, namely ODC, AMD1, ARG2, and SMS, in PCa.

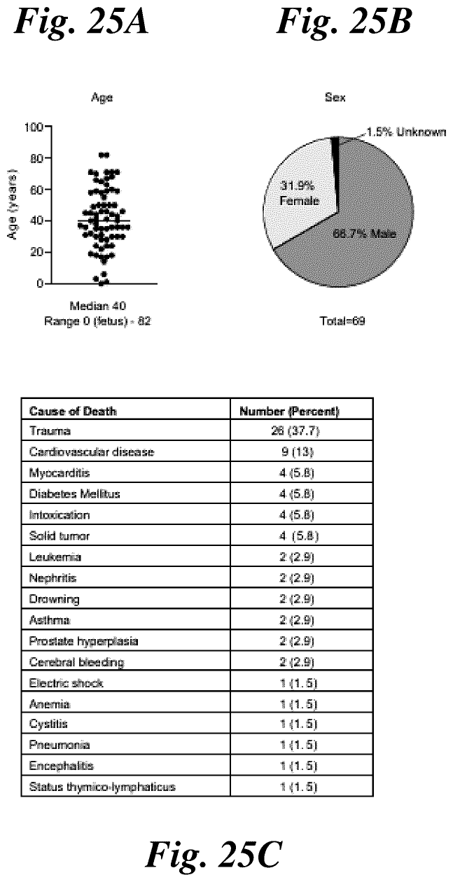

Given that AR regulates polyamine synthesis in the normal prostate of rodents, (18, 19) we suspected that this is a normal function of AR that is common between normal and malignant prostate in humans. In support of this idea, we reanalyzed data from an autopsy study of 69 human subjects with median age of death of 40, representation of males and females, with trauma as the leading cause of death (FIG. 25A-C), in which spermine abundance was measured in 15 tissues, and confirmed that prostate tissue had the highest abundance of spermine of all tissues measured (FIG. 22H) (27). While AR is abundant in prostate epithelium, it is also present in diverse cell types with distinct embryological origins such as T lymphocytes (28). To assess whether AR regulates expression of polyamine synthesis enzymes in cells beyond prostate, we isolated human T lymphocytes from peripheral blood of 5 donors, treated unstimulated or CD3/CD28-stimulated cells with vehicle control, SPA, or the AR inhibitor enzalutamide for 24 hours, and measured ODC. AR expression was low but detectable in all donors, however ODC was only detectable in CD3/CD28-stimulated cells (FIG. 22I). Notably SPA increased ODC in some donors but not all, while enzalutamide decreased ODC in all donors (FIG. 22J). Given these experiments were performed in media containing 10% fetal bovine serum, known to contain castrate-levels of androgens (29), we suspect the low amount of AR expressed was near-saturated by the low amount of androgen present in the media, which may explain why SPA did not reliably increase ODC while AR inhibition reliably reduced its expression. Thus, the AR seems to regulate ODC abundance across diverse human cell types.

### 4.2.3 MYC Antagonizes AR-Stimulated Expression of ODC and AMD1

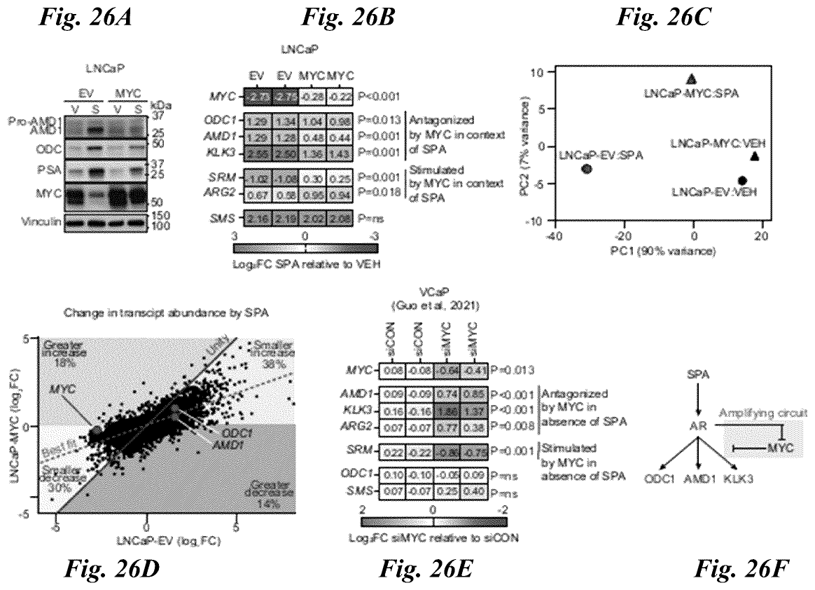

MYC can induce ODC1 transcription via association with a MYC binding site in intron 1 of the ODC1 gene (30-32), in contexts outside of PCa. Similarly, MYC was previously shown to increase AMD1 transcription (33). Yet we observed that SPA increased ODC and AMD1 in LNCaP and VCaP cells despite decreasing MYC (FIG. 22B). To assess the role of MYC in driving expression of ODC and AMD1 in the context of SPA, we treated LNCaP cells stably expressing an empty vector (EV) or MYC with vehicle control or SPA and assessed ODC and AMD1 abundance. Forced constitutive expression of MYC reduced the stimulation of ODC and AMD1 compared with EV control cells following SPA treatment, in a similar manner to PSA (FIG. 26A). RNAseq of these cells confirmed that ODC1 and AMD1 were stimulated by SPA and antagonized by MYC, in a similar manner to KLK3 (FIG. 26B). In contrast, SRM was reduced by SPA and stimulated by MYC, ARG2 was stimulated by SPA and further stimulated by MYC, while SMS was stimulated by SPA but unaffected by MYC (FIG. 26B). This surprising reduction of ODC and AMD1 by MYC may be specific to cells that express AR, in which MYC has been described to antagonize the transcriptional activity of AR, theorized to occur through co-factor redistribution when MYC is high leading to RNA Pol II pausing at promoters of AR-regulated genes (34, 35). Indeed, the constitutive expression of MYC globally tempered transcriptional changes by SPA by principal component analysis (FIG. 26C) and by comparison of the fold change of each transcript by SPA in each cell line (FIG. 26D). In this comparison, the line of best fit was found to have a shallower slope than the line of unity, with more transcripts showing a smaller change rather than greater change by SPA in cells with constitutive MYC (68% versus 32%) (FIG. 26D). Notably, siRNA-mediated knockdown of MYC in VCAP cells resulted in increased AMD1 and KLK3 and did not change ODC1 (FIG. 26E) (re-analyzed data from Guo et al. (36)), suggesting that MYC does not stimulate transcription of AMD1 and ODC1 in AR-positive cells and in fact can negatively regulate their expression by antagonizing transcriptional activity of the AR (FIG. 26F). It is plausible that SPA failed to increase AMD1 abundance in LAPC4 and 22Rv1 cells (FIG. 22B) due to failure to downregulate MYC and alleviate its antagonism of AR stimulation of AMD1. Therefore, the AR, more than MYC, is the primary driver of ODC1 and AMD1 in PCa.

### 4.2.4 ODC Inhibition Enhances Growth Suppression of PCa Models by SPA

To assess whether induction of ODC and polyamine synthesis by SPA has functional significance with regard to growth suppression of PCa by SPA, we utilized a specific and clinically-utilized inhibitor of ODC, difluoromethylornithine (DFMO). DFMO decreased colony formation of LNCaP and VCaP cells treated with androgen in vitro (FIG. 3A-FIG. 3B) and tumor growth of MycCaP-CR tumors growing in castrated mice treated with SPA via subcutaneous testosterone pellets (FIG. 3C). This suggests that induction of polyamine synthesis by SPA may facilitate resistance to treatment and inhibition of this pathway has potential to enhance the efficacy of BAT.

### 4.2.5 ODC Inhibition Enhances Downregulation of MYC by SPA by Depleting SAM

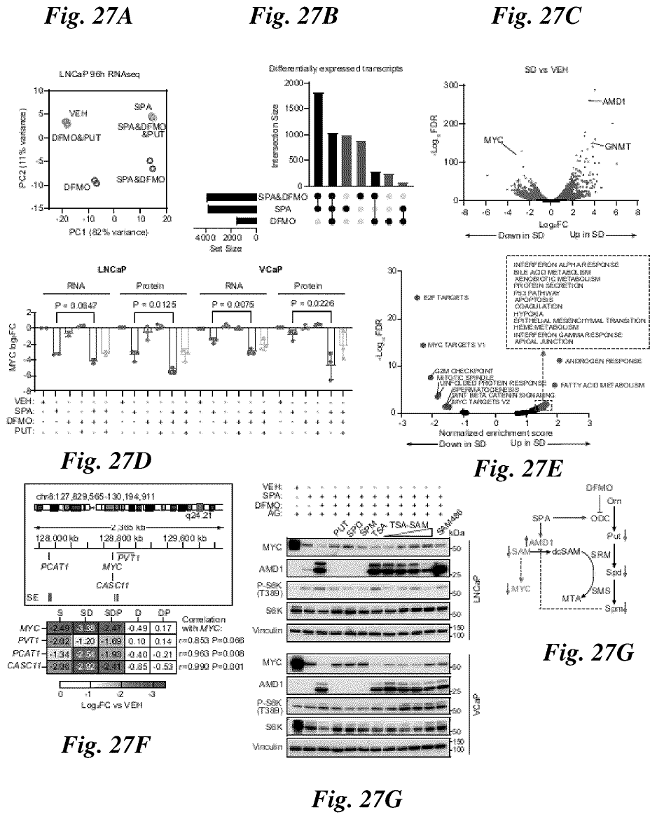

To explore mechanisms by which DFMO enhances growth suppression by SPA, we performed RNAseq of LNCaP cells treated with (1) VEH, (2) SPA, (3) DFMO, (4) SPA & DFMO, (5) DFMO & putrescine, and (6) SPA & DFMO & putrescine for 96 hours. The transcriptome of cells treated with SPA & DFMO, SPA monotherapy, or DFMO monotherapy were markedly different from VEH-treated cells by principal component analysis, with those from cells treated with SPA & DFMO showing the greatest change (FIG. 27A). Notably, putrescine supplementation reversed transcriptional effects of DEMO (when used as monotherapy or in combination with SPA) (FIG. 27A), indicating that transcriptional changes due to DFMO seem to be on-target effects of ODC inhibition and consequent putrescine depletion. In general, cellular changes due to combination therapies can be driven by one of the therapies (and occur even with monotherapy) or they can be driven by an interaction between the therapies (and only occur with combination therapy). Cells treated with SPA or SPA & DFMO each had about 4000 differentially expressed transcripts when compared with cells treated with VEH, while cells treated with DFMO had fewer (FIG. 27B). While there was definite overlap of transcripts differentially expressed in cells treated with SPA & DFMO and cells treated with each monotherapy, we also observed that about 1000 transcripts were exclusively differentially expressed in SPA & DFMO and many transcripts were exclusively altered in either SPA or DFMO monotherapy (FIG. 27B). This suggests that SPA & DFMO combination therapy has a unique effect on the transcriptome of PCa, that is not simply the summation of effects of each monotherapy.

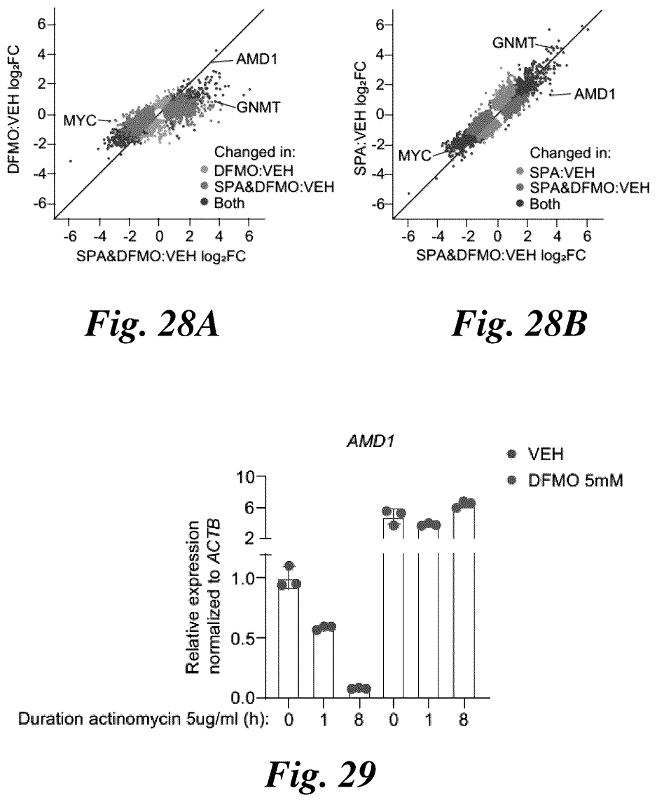

Next, we assessed how SPA & DFMO combination therapy alters the transcriptome of PCa. We noted that MYC was markedly reduced by combination therapy to a greater extent than either monotherapy, which occurred in multiple cell lines and translated to reduced MYC protein abundance and expression of MYC target genes (FIG. 27C-FIG. 27E). We and others previously determined that efficacy of SPA monotherapy in PCa models and patients is due in part to MYC downregulation, which occurs in conjunction with downregulation of PVT1 and PCAT1 located together with MYC in the 8q24 topologically associated domain under control of a centromeric super-enhancer (SE) near PCAT1 (3, 16) (FIG. 27F). Abundance of PCAT1 and CASC11 tightly correlated with MYC across conditions (FIG. 27F), suggesting that the mechanism by which DFMO further reduces MYC may relate to furthered diminished activity of the PCAT1 SE. We also noted that combination therapy markedly increased abundance of AMD1 and GNMT (glycine N-methyltransferase) (FIG. 27C), two enzymes that consume S-adenosylmethionine (SAM). In contrast to MYC, for which both therapies contributed to its differential expression, AMD1 increase was driven by DFMO and GNMT increase was driven by SPA, as their change in abundance was similar in monotherapy-treated cells to combination therapy-treated cells (FIG. 28A-FIG. 28B). These alterations may have been expected based on prior studies that indicate that AR can increase GNMT leading to accumulation of its product sarcosine in PCa (42) and polyamine depletion by DFMO can increase transcription of AMD1 in prostate tissue (43). Moreover, we observed that DFMO markedly increased stability of the AMD1 transcript, which its half-life increasing from about 1 hour in VEH-treated LNCaP cells to >8 hours in DFMO-treated cells (FIG. 29). We hypothesized that the increased expression of AMD1 and GNMT may lead to their increased activity and consequent depletion of their substrate SAM. The bioavailability of SAM can have consequences on the transcriptome by modifying chromatin accessibility through DNA and histone methylation. Indeed, we observed that supplementation of SAM was sufficient to rescue MYC in LNCaP and VCaP cells treated with SPA and DFMO to the level of SPA monotherapy, and this effect could be phenocopied by inhibition of AMD1 using the inhibitor SAM486 (FIG. 27G). Previous studies have suggested that depletion of SAM can inhibit mTORC1 signaling through SAMTOR (44) which can lead to downregulation of MYC, however we did not observe decreased mTORC1 activity (as assessed by phosphorylation of S6K) with combination treatment (FIG. 27G), suggesting that altered signaling through mTORC1 is unlikely to contribute to the downregulation of MYC in this system. Altogether, these data suggest that DFMO enhances downregulation of MYC by SPA through stimulation of AMD1 to deplete SAM, which may alter SE activity on 8q24.

### 4.3 Summary

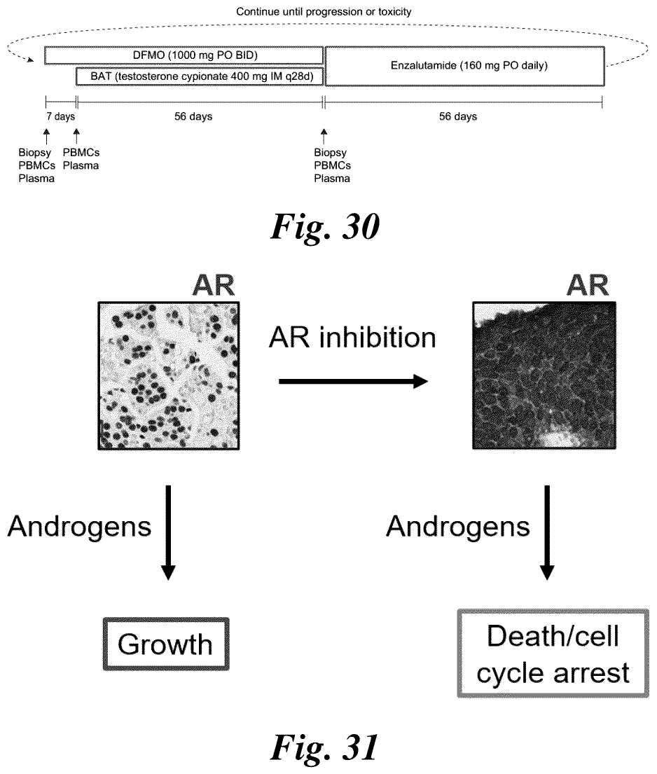

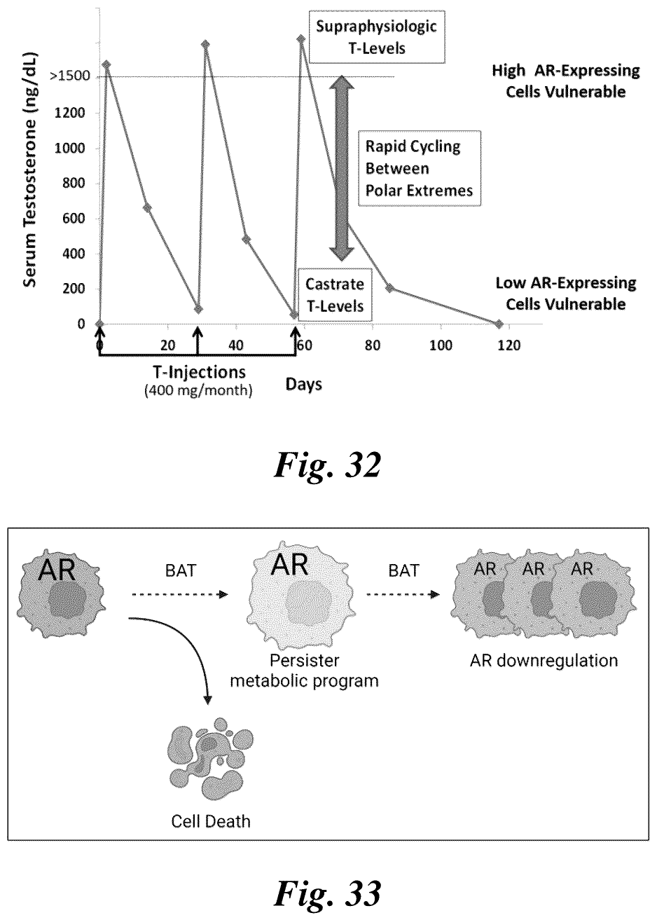

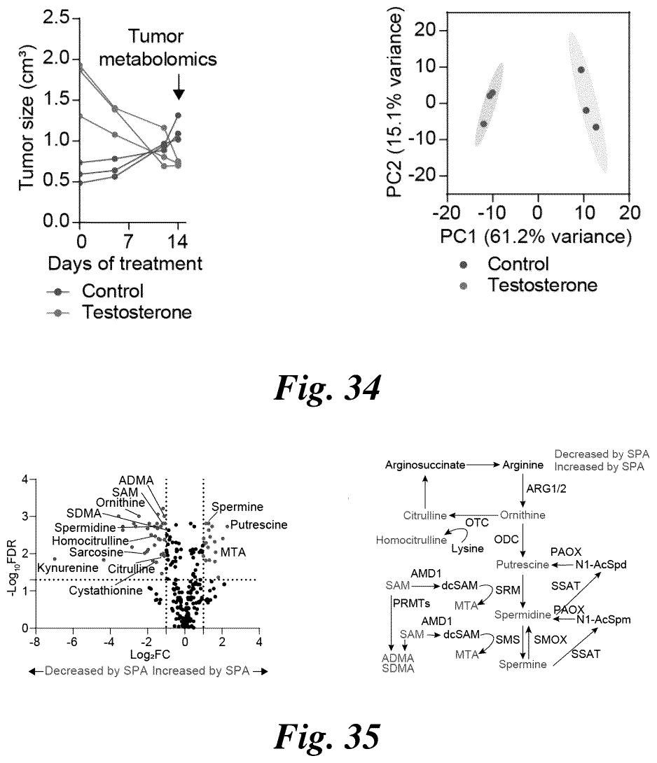

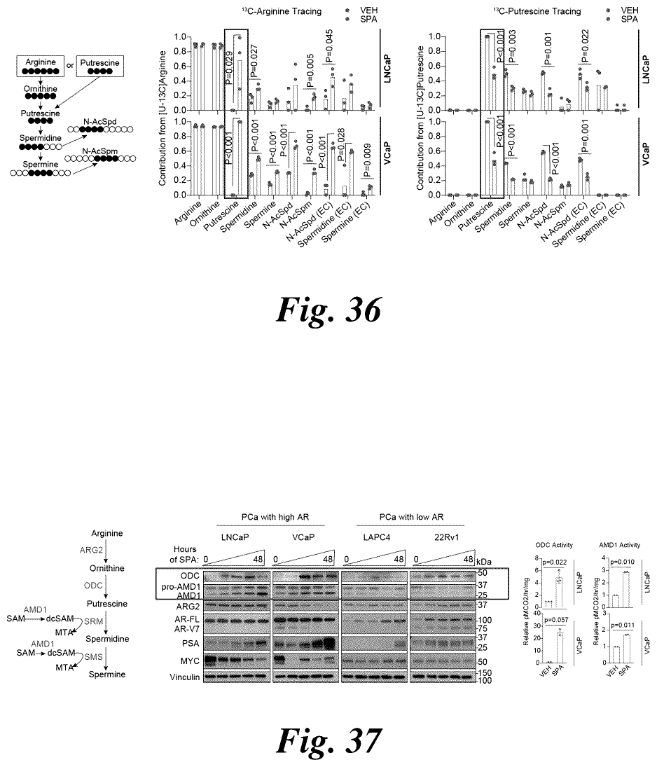

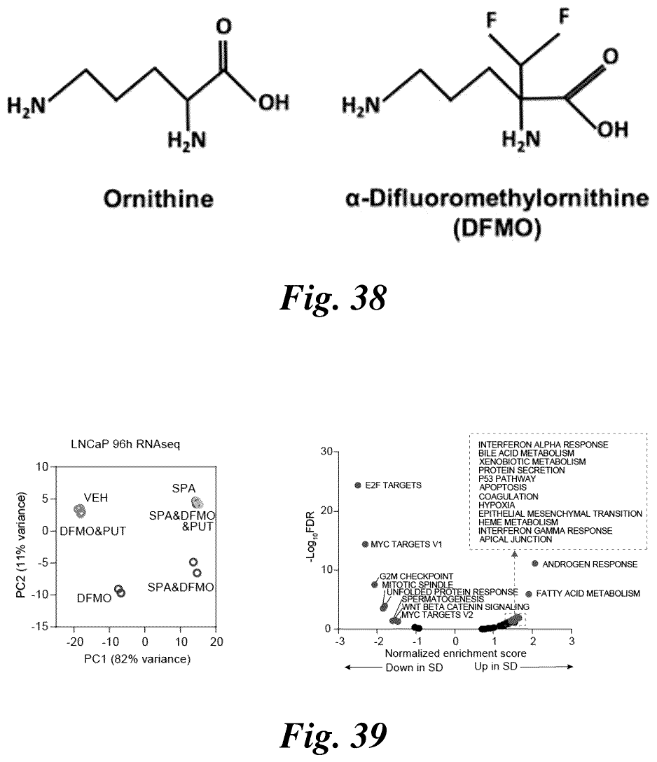

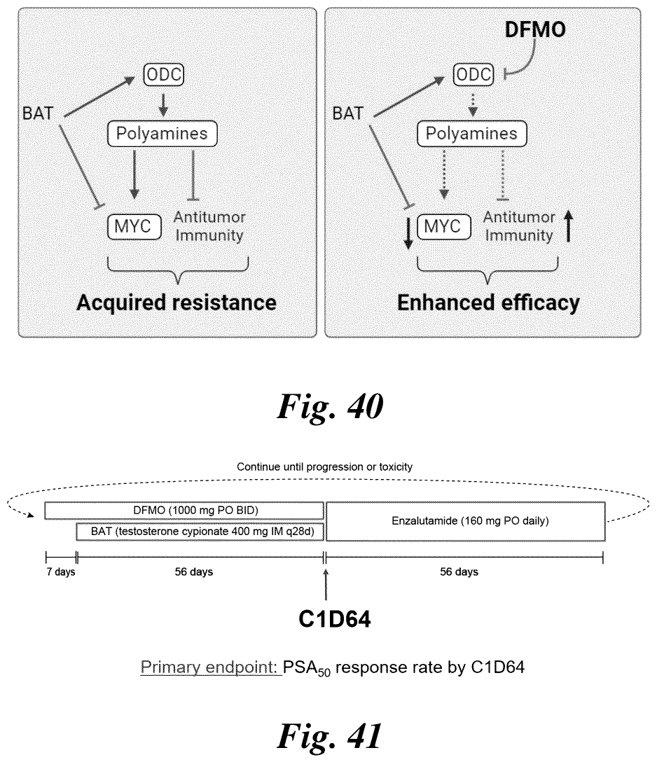

These data suggest that AR drives polyamine synthesis in PCa models treated with SPA. This appears to be a feature of the AR that is not specific to cancer cells and also occurs in normal prostate and T cells. While MYC can potently stimulate polyamine synthesis in other contexts, the AR is a stronger stimulator of polyamine synthesis in PCa, in which MYC paradoxically can decrease expression of ODC and AMD1 by antagonizing AR transcriptional activity. The induction of polyamines by AR appears to be functionally important in PCa models, as inhibition of this pathway by DFMO can enhance growth repression by SPA. Mechanistically, this enhanced growth suppression by combination therapy of SPA and DFMO occurs in part through enhanced downregulation of MYC. The downregulation of MYC appears to be a consequence of enhanced activity of AMD1 by DFMO, which leads to depletion of the methyl donor SAM. Future studies will delineate precisely how depletion of SAM is able to suppress MYC but our data suggests that it occurs independent of mTORC1 and may modify chromatin accessibility of the SE on 8q24. These data provide strong rationale for a clinical trial testing whether administration of DFMO in combination with BAT can improve efficacy of BAT for patients with metastatic castration-resistant PCa (FIG. 30).

### 4.4 References Example 4

1. Kumar, R., Sena, L. A., Denmeade, S. R., and Kachhap, S. (2022). The testosterone paradox of advanced prostate cancer: mechanistic insights and clinical implications. Nat. Rev. Urol. 10.1038/s41585-022-00686-y.
2. Isaacs, J. T., D'Antonio, J. M., Chen, S., Antony, L., Dalrymple, S. P., Ndikuyeze, G. H., Luo, J., and Denmeade, S. R. (2012). Adaptive auto-regulation of androgen receptor provides a paradigm shifting rationale for bipolar androgen therapy (BAT) for castrate resistant human prostate cancer. Prostate 72, 1491-1505.
3. Sena, L. A., Kumar, R., Sanin, D. E., Thompson, E. A., Rosen, D. M., Dalrymple, S. L., Antony, L., Yang, Y., Gomes-Alexandre, C., Hicks, J. L., et al. (2022). Prostate cancer androgen receptor activity dictates efficacy of bipolar androgen therapy through MYC. J. Clin. Invest. 10.1172/JCI162396.
4. Denmeade, S. R., Wang, H., Agarwal, N., Smith, D. C., Schweizer, M. T., Stein, M. N., Assikis, V., Twardowski, P. W., Flaig, T. W., Szmulewitz, R. Z., et al. (2021). TRANSFORMER: A randomized phase II study comparing bipolar androgen therapy versus enzalutamide in asymptomatic men with castration-resistant metastatic prostate cancer. J. Clin. Oncol., JCO2002759.
5. Lupien, M., Eeckhoute, J., Meyer, C. A., Wang, Q., Zhang, Y., Li, W., Carroll, J. S., Liu, X. S., and Brown, M. (2008). FoxA1 translates epigenetic signatures into enhancer-driven lineage-specific transcription. Cell 132, 958-970.
6. Sharma, N. L., Massie, C. E., Ramos-Montoya, A., Zecchini, V., Scott, H. E., Lamb, A. D., MacArthur, S., Stark, R., Warren, A. Y., Mills, I. G., et al. (2013). The androgen receptor induces a distinct transcriptional program in castration-resistant prostate cancer in man. Cancer Cell 23, 35-47.
7. Pomerantz, M. M., Li, F., Takeda, D. Y., Lenci, R., Chonkar, A., Chabot, M., Cejas, P., Vazquez, F., Cook, J., Shivdasani, R. A., et al. (2015). The androgen receptor cistrome is extensively reprogrammed in human prostate tumorigenesis. Nat. Genet. 47, 1346-1351.
8. Massie, C. E., Lynch, A., Ramos-Montoya, A., Boren, J., Stark, R., Fazli, L., Warren, A., Scott, H., Madhu, B., Sharma, N., et al. (2011). The androgen receptor fuels prostate cancer by regulating central metabolism and biosynthesis. EMBO J. 30, 2719-2733.
9. Crowell, P. D., Giafaglione, J. M., Jones, A. E., Nunley, N. M., Hashimoto, T., Delcourt, A. M. L., Petcherski, A., Agrawal, R., Bernard, M. J., Diaz, J. A., et al. (2023). MYC is a regulator of androgen receptor inhibition-induced metabolic requirements in prostate cancer. Cell Rep. 42, 113221.
10. Sena, L. A., and Denmeade, S. R. (2021). Fatty acid synthesis in prostate cancer: vulnerability or epiphenomenon? Cancer Res., canres. 1392.2021.
11. MacLean, H. E., Chiu, W. S. M., Notini, A. J., Axell, A.-M., Davey, R. A., McManus, J. F., Ma, C., Plant, D. R., Lynch, G. S., and Zajac, J. D. (2008). Impaired skeletal muscle development and function in male, but not female, genomic androgen receptor knockout mice. FASEB J. 22, 2676-2689.
12. Corona, G., Giagulli, V. A., Maseroli, E., Vignozzi, L., Aversa, A., Zitzmann, M., Saad, F., Mannucci, E., and Maggi, M. (2016). Testosterone supplementation and body composition: results from a meta-analysis of observational studies. J. Endocrinol. Invest. 39, 967-981.
13. Marshall, C. H., Tunacao, J., Danda, V., Tsai, H.-L., Barber, J., Gawande, R., Weiss, C. R., Denmeade, S. R., and Joshu, C. (2021). Reversing the effects of androgen-deprivation therapy in men with metastatic castration-resistant prostate cancer. BJU Int. 128, 366-373.
14. Martínez-Reyes, I., and Chandel, N. S. (2020). Mitochondrial TCA cycle metabolites control physiology and disease. Nat. Commun. 11, 102.
15. Mann, T. (1954). The biochemistry of semen (Methuen).
16. Zhu, Y., Dalrymple, S. L., Coleman, I., Zheng, S. L., Xu, J., Hooper, J. E., Antonarakis, E. S., De Marzo, A. M., Meeker, A. K., Nelson, P. S., et al. (2020). Role of androgen receptor splice variant-7 (AR-V7) in prostate cancer resistance to 2nd-generation androgen receptor signaling inhibitors. Oncogene 39, 6935-6949.
17. Casero, R. A., Jr, Murray Stewart, T., and Pegg, A. E. (2018). Polyamine metabolism and cancer: treatments, challenges and opportunities. Nat. Rev. Cancer 18, 681-695.
18. Pegg, A. E., and Williams-Ashman, H. G. (1968). Rapid effects of testosterone in prostatic polyamine-synthesizing enzyme systems. Biochem. J. 109, 32P-33P.
19. Pegg, A. E., Lockwood, D. H., and Williams-Ashman, H. G. (1970). Concentrations of putrescine and polyamines and their enzymic synthesis during androgen-induced prostatic growth. Biochem. J. 117, 17-31.
20. Jänne, O. A., Kontula, K. K., Isomaa, V. V., and Bardin, C. W. (1984). Ornithine decarboxylase mRNA in mouse kidney: a low abundancy gene product regulated by androgens with rapid kinetics. Ann. N. Y. Acad. Sci. 438, 72-84.
21. Pajunen, A. E., Isomaa, V. V., Janne, O. A., and Bardin, C. W. (1982). Androgenic regulation of ornithine decarboxylase activity in mouse kidney and its relationship to changes in cytosol and nuclear androgen receptor concentrations. J. Biol. Chem. 257, 8190-8198.
22. Isomaa, V. V., Pajunen, A. E., Bardin, C. W., and Jänne, O. A. (1983). Ornithine decarboxylase in mouse kidney. Purification, characterization, and radioimmunological determination of the enzyme protein. J. Biol. Chem. 258, 6735-6740.
23. Betts, A. M., Waite, I., Neal, D. E., and Robson, C. N. (1997). Androgen regulation of ornithine decarboxylase in human prostatic cells identified using differential display. FEBS Lett. 405, 328-332.
24. Abida, W., Cyrta, J., Heller, G., Prandi, D., Armenia, J., Coleman, I., Cieslik, M., Benelli, M., Robinson, D., Van Allen, E. M., et al. (2019). Genomic correlates of clinical outcome in advanced prostate cancer. Proc. Natl. Acad. Sci. U.S.A 116, 11428-11436.
25. Rajan, P., Sudbery, I. M., Villasevil, M. E. M., Mui, E., Fleming, J., Davis, M., Ahmad, I., Edwards, J., Sansom, O. J., Sims, D., et al. (2014). Next-generation sequencing of advanced prostate cancer treated with androgen-deprivation therapy. Eur. Urol. 66, 32-39.
26. Hoadley, K. A., Yau, C., Hinoue, T., Wolf, D. M., Lazar, A. J., Drill, E., Shen, R., Taylor, A. M., Cherniack, A. D., Thorsson, V., et al. (2018). Cell-of-origin patterns dominate the molecular classification of 10,000 tumors from 33 types of cancer. Cell 173, 291-304.e6.
27. Hamalainen, R. (1941). Uber die quantitative bestimmung des spermins im organismus und sein vorkommen in menschlichen geweben und korperflussingkeiten. Acta Soc. Med 23, 97-165.
28. Guan, X., Polesso, F., Wang, C., Sehrawat, A., Hawkins, R. M., Murray, S. E., Thomas, G. V., Caruso, B., Thompson, R. F., Wood, M. A., et al. (2022). Androgen receptor activity in T cells limits checkpoint blockade efficacy. Nature 606, 791-796.
29. Sedelaar, J. P. M., and Isaacs, J. T. (2009). Tissue culture media supplemented with 10% fetal calf serum contains a castrate level of testosterone. Prostate 69, 1724-1729.
30. Wagner, A. J., Meyers, C., Laimins, L. A., and Hay, N. (1993). c-Myc induces the expression and activity of ornithine decarboxylase. Cell Growth Differ. 4, 879-883.
31. Peña, A., Reddy, C. D., Wu, S., Hickok, N. J., Reddy, E. P., Yumet, G., Soprano, D. R., and Soprano, K. J. (1993). Regulation of human ornithine decarboxylase expression by the c-Myc.Max protein complex. J. Biol. Chem. 268, 27277-27285.
32. Bello-Fernandez, C., Packham, G., and Cleveland, J. L. (1993). The ornithine decarboxylase gene is a transcriptional target of c-Myc. Proc. Natl. Acad. Sci. U.S.A 90, 7804-7808.
33. Marinkovic, D., Marinkovic, T., Kokai, E., Barth, T., Möller, P., and Wirth, T. (2004). Identification of novel Myc target genes with a potential role in lymphomagenesis. Nucleic Acids Res. 32, 5368-5378.
34. Barfeld, S. J., Urbanucci, A., Itkonen, H. M., Fazli, L., Hicks, J. L., Thiede, B., Rennie, P. S., Yegnasubramanian, S., DeMarzo, A. M., and Mills, I. G. (2017). C-Myc antagonises the transcriptional activity of the androgen receptor in prostate cancer affecting key gene networks. EBioMedicine 18, 83-93.
35. Qiu, X., Boufaied, N., Hallal, T., Feit, A., de Polo, A., Luoma, A. M., Alahmadi, W., Larocque, J., Zadra, G., Xie, Y., et al. (2022). MYC drives aggressive prostate cancer by disrupting transcriptional pause release at androgen receptor targets. Nat. Commun. 13, 2559.
36. Guo, H., Wu, Y., Nouri, M., Spisak, S., Russo, J. W., Sowalsky, A. G., Pomerantz, M. M., Wei, Z., Korthauer, K., Seo, J.-H., et al. (2021). Androgen receptor and MYC equilibration centralizes on developmental super-enhancer. Nat. Commun. 12, 7308.
37. Pegg, A. E. (2016). Functions of polyamines in mammals. J. Biol. Chem. 291, 14904-14912.
38. Jänne, O. A., Crozat, A., Palvimo, J., and Eisenberg, L. M. (1991). Androgen-regulation of ornithine decarboxylase and S-adenosylmethionine decarboxylase genes. J. Steroid Biochem. Mol. Biol. 40, 307-315.
39. Bai, G., Kasper, S., Matusik, R. J., Rennie, P. S., Moshier, J. A., and Krongrad, A. (1998). Androgen regulation of the human ornithine decarboxylase promoter in prostate cancer cells. J. Androl. 19, 127-135.
40. Asangani, I. A., Dommeti, V. L., Wang, X., Malik, R., Cieslik, M., Yang, R., Escara-Wilke, J., Wilder-Romans, K., Dhanireddy, S., Engelke, C., et al. (2014). Therapeutic targeting of BET bromodomain proteins in castration-resistant prostate cancer. Nature 510, 278-282.
41. Pegg, A. E. (2006). Regulation of ornithine decarboxylase. J. Biol. Chem. 281, 14529-14532.
42. Sreekumar, A., Poisson, L. M., Rajendiran, T. M., Khan, A. P., Cao, Q., Yu, J., Laxman, B., Mehra, R., Lonigro, R. J., Li, Y., et al. (2009). Metabolomic profiles delineate potential role for sarcosine in prostate cancer progression. Nature 457, 910-914.
43. Shirahata, A., and Pegg, A. E. (1986). Increased content of mRNA for a precursor of S-adenosylmethionine decarboxylase in rat prostate after treatment with 2-difluoromethylornithine. J. Biol. Chem. 261, 13833-13837.
44. Gu, X., Orozco, J. M., Saxton, R. A., Condon, K. J., Liu, G. Y., Krawczyk, P. A., Scaria, S. M., Harper, J. W., Gygi, S. P., and Sabatini, D. M. (2017). SAMTOR is an S-adenosylmethionine sensor for the mTORC1 pathway. Science 358, 813-818.
45. Pham, V. N., Bruemmer, K. J., Toh, J. D. W., Ge, E. J., Tenney, L., Ward, C. C., Dingler, F. A., Millington, C. L., Garcia-Prieto, C. A., Pulos-Holmes, M. C., et al. (2023). Formaldehyde regulates S-adenosylmethionine biosynthesis and one-carbon metabolism. Science 382, eabp9201.
46. Villa, E., Sahu, U., O'Hara, B. P., Ali, E. S., Helmin, K. A., Asara, J. M., Gao, P., Singer, B. D., and Ben-Sahra, I. (2021). mTORC1 stimulates cell growth through SAM synthesis and m6A mRNA-dependent control of protein synthesis. Mol. Cell 81, 2076-2093.e9.
47. Mavrakis, K. J., McDonald, E. R., 3rd, Schlabach, M. R., Billy, E., Hoffman, G. R., deWeek, A., Ruddy, D. A., Venkatesan, K., Yu, J., McAllister, G., et al. (2016). Disordered methionine metabolism in MTAP/CDKN2A-deleted cancers leads to dependence on PRMT5. Science 351, 1208-1213.
48. Crozat, A. (1992). Comparison of androgen regulation of ornithine decarboxylase and S-adenosylmethionine decarboxylase gene expression in rodent kidney and accessory sex organs. Endocrinology 130, 1131-1144.

### Example 5

Enhancing Efficacy of Bipolar Androgen Therapy by Targeting Polyamine Synthesis in Patients with Metastatic CRPC: The APEX Trial

Six clinical trials of BAT for patients with advanced prostate cancer have been completed. Schweizer et al (2016) Prostate; Teply et al (2018) Lancet Oncol; Markowski et al (2021) Eur Urol; Sena et al (2021) Eur J Cancer; Denmeade et al (2021) J Clin Oncol; Schweizer et al (2023) PCAN; Sena et al (2023) Eur J Cancer; Markowski et al (2024) Nat Comm.

BAT has good safety profile among patients with asymptomatic disease and has antitumor effect as monotherapy (approximately 30% objective response rate for patients with mCRPC, with median PFS approximately 6 months). BAT also appears to sensitize prostate cancer to subsequent AR inhibition. Further, BAT improves quality of life and Is inexpensive. Accordingly, BAT can be a new platform for testing of combination therapies.

Without wishing to be bound to any one particular theory, it is thought that BAT induces a persister metabolic program that enables development of acquired resistance. (See FIG. 33).

Objections of this Example include, but are not limited to: (a) confirmation that DFMO inhibits ODC and reduces tumor polyamine abundance despite BAT; (b) determination of the effect of DFMO and BAT on: (i) MYC and MYC signaling; (ii) circulating and tumor-infiltrating immune cell abundance and phenotype by scRNAseq; (iii) diversity of gut microflora and metabolite abundance; and (iv) CtDNA abundance; and (c) determination of whether any of the above associates with efficacy.

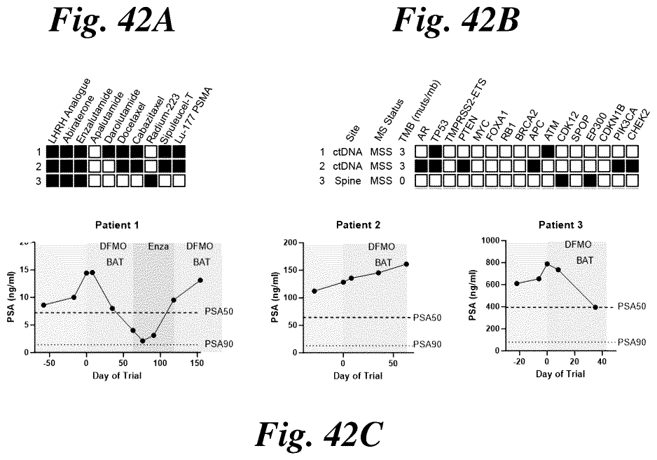

Referring now to FIG. 42A-FIG. 42C, the first three patients enrolled on the trial have metastatic prostate cancer refractory to numerous systemic therapies (FIG. 42A) with common genomic alterations (FIG. 42B). Black boxes indicate the therapy was received or a pathogenic genomic alteration was present. The first and third patients on trial met the primary endpoint of the study, which is a 50% decline in the serum PSA from C1D1 by C1D64 (FIG. 42). The second patient on the study did not meet the primary endpoint (FIG. 42). All patients remain on study at the time of data collection.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating prostate cancer in a subject in need of treatment thereof, the method comprising administering to the subject: (a) a first dose of a polyamine inhibitor for a first interval at a beginning of a first treatment cycle; (b) a second dose of the polyamine inhibitor in combination with a dose of an androgen, or a derivative thereof, at a second interval during the first treatment cycle; and (c) a dose of an antiandrogen at a third interval during the first treatment cycle.

2. The method of claim 1, wherein the first dose of the polyamine inhibitor is administered for a first interval of about 7±3 days at the beginning of the first treatment cycle.

3. The method of claim 1, wherein the second dose of the polyamine inhibitor in combination with a dose of the androgen, or a derivative thereof, is administered for a second interval of about 56±7 days at a completion of the first interval during the first treatment cycle.

4. The method of claim 3, wherein the second dose of the polyamine inhibitor in combination with a dose of the androgen, or a derivative thereof, is administered for a second interval comprising continuous administration until evidence of prostate cancer disease progression is observed, as evidenced by one or more criteria selected from a rise in serum PSA levels, a worsening of one or more clinical symptoms due to prostate cancer, or a worsening disease on imaging studies, wherein the imaging studies are selected from a computed tomography (CT) scan, magnetic resonance imaging (MRI), a bone scan, PSMA-based positron emission tomography (PET) imaging, and a ¹⁸F-fluciclovine PET/CT scan.

5. (canceled)

6. The method of claim 1, wherein the dose of the antiandrogen is administered at a third interval of about 56±7 days at a completion of the second interval during the first treatment cycle.

7. The method of claim 6, wherein the dose of the antiandrogen is administered at a completion of the second interval during the first treatment cycle at a third interval comprising continuous administration until evidence of prostate cancer disease progression is observed as evidenced by one or more criteria selected from a rise in serum PSA levels, a worsening of clinical symptoms due to prostate cancer, or worsening disease on imaging studies, wherein the imaging studies are selected form a CT scan, an MRI, a bone scan, PSMA-based PET imaging, and a ¹⁸F-fluciclovine PET/CT scan.

8. (canceled)

9. The method of claim 1, further comprising discontinuing administration of the second dose of the polyamine inhibitor in combination with the dose of an androgen, or a derivative thereof, at a completion of the second interval during the first treatment cycle.

10. The method of claim 1, further comprising discontinuing administration of the dose of the antiandrogen at a completion of the third interval during the first treatment cycle.

11. The method of claim 1, wherein the first dose and the second dose of the polyamine inhibitor are each about 1000 mg/day in both the first interval and the second interval of the first treatment cycle.

12. The method of claim 1, wherein the dose of the androgen, or an androgen derivative thereof, comprises a supraphysiologic level of testosterone or the androgen derivative.

13. The method of claim 12, wherein the supraphysiological level of androgen (SPA), or a derivative thereof, comprises a serum concentration of androgen between about 3 to about 10 times a normal serum concentration of androgen of the subject.

14. The method of claim 12, wherein the androgen, or an androgen derivative thereof, is given in sufficient quantity to produce a supraphysiologic level of androgen (SPA) comprising a serum concentration of androgen or greater than about 1,500 ng/dL or a concentration of an androgen derivative that is equivalent to a level of androgen or greater than about 1,500 ng/dL.

15. The method of claim 12, wherein the dose of the androgen is between about 400 mg to about 500 mg over a 28-day treatment cycle when administered via intramuscular injection (IM) or between about 15 mg to about 20 mg per day when administered by a method other than IM.

16. The method of claim 15, wherein the method other than IM is selected from transdermal, buccal, and intranasal.

17. The method of claim 1, wherein the dose of the antiandrogen is between about 50 mg/day to about 1200 mg/day.

18. The method of claim 1, wherein the method further comprises administering one or more androgen synthesis inhibitors.

19. The method of claim 18, wherein the one or more androgen synthesis inhibitors are selected from the group consisting of a CYP17A1 inhibitor, a CYP11A1 (P450scc) inhibitor, a 5α-Reductase inhibitor, and combinations thereof.

20. The method of claim 19, wherein the one or more androgen synthesis inhibitors are selected from the group consisting of abiraterone acetate, ketoconazole, seviteronel, aminoglutethimide, alfatradiol, dutasteride, epristeride, finasteride, and combinations thereof.

21. The method of claim 1, wherein the polyamine inhibitor is selected from the group consisting of a polyamine synthesis inhibitor, a polyamine analog, a polyamine uptake inhibitor, and combinations thereof.

22. The method of claim 1, wherein the polyamine inhibitor is administered alone or as a complex in a carrier.

23. The method of claim 22, wherein the carrier comprises a liposome or a nanoparticle.

24. The method of claim 22, wherein the polyamine inhibitor is administered intravenously, subcutaneously, or orally.

25. The method of claim 1, wherein the polyamine inhibitor is selected from an ornithine decarboxylase inhibitor or a polyamine analog.

26. The method of claim 25, wherein the ornithine decarboxylase inhibitor comprises difluoromethylornithine (DFMO) or wherein the polyamine analog comprises SBP101 (Ivospemin).

27. (canceled)

28. The method of claim 1, wherein the androgen, or a derivative thereof, comprises an ester of testosterone or an ester of dihydrotestosterone.

29. The method of claim 28, wherein the ester of testosterone or the ester of dihydrotestosterone is selected from a cypionate, enanthate, propionate, butyrate, and undecanoate ester of testosterone or dihydrotestosterone.

30. The method of claim 29, wherein the ester of testosterone is testosterone cypionate or testosterone enanthate.

31. (canceled)

32. The method of claim 1, wherein the method comprises administering the androgen, or a derivative thereof, orally, transdermally, transbuccally, intranasal, or intramuscular injection.

33. The method of claim 1, wherein the antiandrogen is selected from the group consisting of bicalutamide, flutamide, nilutamide, apalutamide, darolutamide, enzalutamide, cyproterone acetate, proxalutamide, cimetidine, and topilutamide.

34-35. (canceled)

36. The method of claim 1, further comprising administering one or more agents that are ligand-independent inhibitors of a function of an androgen receptor.

37. The method of claim 36, wherein the ligand-independent inhibitors are selected from the group consisting of N-terminal domain-targeted androgen receptor inhibitors, DNA-binding domain-targeted androgen receptor inhibitors, androgen receptor mRNA inhibitors, and agents that produce degradation of androgen receptor protein.

38. The method of claim 37, wherein the androgen receptor inhibitor comprises a proteolysis targeting chimera (PROTAC) that can degrade the androgen receptor protein.

39. The method of claim 1, wherein the treatment:
(a) inhibits growth of castration resistant prostate cancer cells;
(b) blocks production of one or more polyamines;
(c) decreases expression of an oncogene MYC;
(d) augments an antitumor immune response.

40. (canceled)

41. The method of claim 39, wherein the one or more polyamines are selected from putrescine, spermidine, and spermine.

42. The method of claim 39, wherein the blocking of the production of one or more polyamines includes blocking an ornithine decarboxylase (ODC) enzyme with DFMO.

43-44. (canceled)

45. The method of claim 1, wherein the method further comprises concurrently administering an androgen deprivation therapy (ADT) to the subject.

46. The method of claim 45, wherein the ADT comprises surgical castration or administering a luteinizing hormone-releasing hormone (LHRH) agonist or a LHRH antagonist to the subject.

47. The method of claim 46, wherein the LHRH agonist is selected from the group consisting of leuprolide, goserelin, triptorelin, and histrelin or wherein the LHRH antagonist is selected from the group consisting of degarelix and relugolix.

48. (canceled)

49. The method of claim 1, wherein the method further comprises administering immune checkpoint blockade therapy to the subject if the subject exhibits clinical and/or radiographic progression.

50. The method of claim 49, wherein the immune checkpoint blockade therapy comprises administering an anti-PD1/PDL1 antibody or an anti-CTLA4 antibody.

51. The method of claim 50, wherein the anti-PD1/PDL1 antibody is selected from the group consisting of pembrolizumab, nivolumab, and atezolizumab or wherein the anti-CTLA4 antibody comprises ipilimumab.

52. (canceled)

53. The method of claim 1, wherein the subject has progressive prostate cancer after treatment with abiraterone or an antiandrogen in combination with androgen deprivation therapy (ADT) as an initial therapy or as a second-line therapy after development of resistance to primary ADT.

54. The method of claim 1, wherein the prostate cancer comprises castration resistant metastatic prostate cancer.

55. The method of claim 1, wherein the subject is asymptomatic.

56. The method of claim 1, wherein the subject is symptomatic.
